(19) United States
(12) Patent Application Publication
(10) Pub. No.: US 2019/0194219 A1
(43) Pub. Date: Jun. 27, 2019

(54) ORGANIC ELECTROLUMINESCENCE DEVICE AND ELECTRONIC APPARATUS EQUIPPED WITH SAME

(71) Applicant: IDEMITSU KOSAN CO., LTD., Tokyo (JP)

(72) Inventors: Masahiro KAWAMURA, Sodegaura-shi, Chiba (JP); Kazuki NISHIMURA, Sodegaura-shi, Chiba (JP)

(73) Assignee: IDEMITSU KOSAN CO., LTD., Tokyo (JP)

(21) Appl. No.: 16/329,210

(22) PCT Filed: Aug. 28, 2017
(86) PCT No.: PCT/JP2017/030808

§ 371 Date: Feb. 27, 2019

(30) Foreign Application Priority Data
Aug. 30, 2016 (JP) 2016-168508

(51) Int. Cl. C07D 491/147; C07D 495/14; C07D 495/22; C07D 487/04; C07D 403/10; C07D 403/14; C07D 251/24; H01L 51/00; H01L 51/50

(52) U.S. Cl. CPC C07D 491/147; H01L 51/5072; C07D 495/22; C07D 487/04; C07D 403/10; C07D 403/14; C07D 251/24; H01L 51/0072; H01L 51/0073; H01L 51/0074; H01L 51/0054; H01L 51/0067; H01L 51/5016; H01L 51/0085; H01L 51/0087; H01L 51/0088; H01L 51/5056; C07D 495/14

(57) ABSTRACT

An organic electroluminescence device including an anode, a cathode, and an emitting layer between the anode and the cathode, in which the emitting layer comprises a compound represented by the formula (1) and a compound represented by the formula (2).

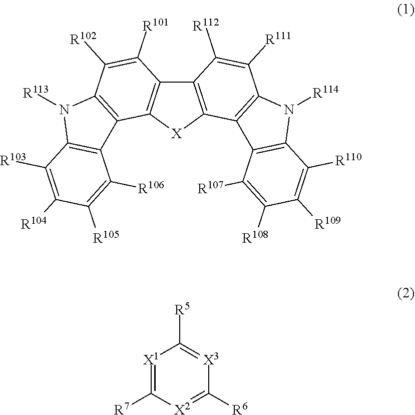

### TECHNICAL FIELD

The present invention relates to an organic electroluminescence device and an electronic apparatus equipped therewith.

### BACKGROUND ART

Patent Literature 1 discloses a compound having a condensed ring structure represented by the general formula below, which is used as a phosphorescent emitting host material.

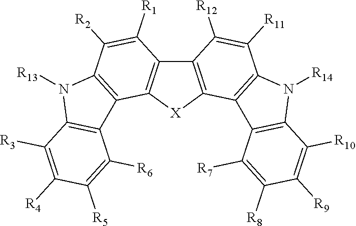

(wherein X is carbon, nitrogen, oxygen, phosphorus, sulfur, silicon, or germanium.)

Patent Literature 2 discloses that an improved lifetime can be achieved while high efficiency is maintained, with the use of a mixed emitting layer using an electron-transporting host material represented by the general formula (1) and a hole-transporting host material represented by the general formula (2).

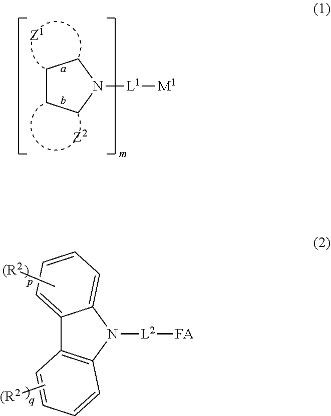

Patent Literature 3 to Patent Literature 5 disclose techniques similar to the technique disclosed in Patent Literature 2, stating that performance is improved by using as an emitting layer a mixture of two types of hosts having different properties.

### CITATION LIST

### Patent Literature

Patent Literature 1 WO 2011/019173

Patent Literature 2 WO 2013/062075

Patent Literature 3 US 2015/0207079

Patent Literature 4 US 2015/0236262

Patent Literature 5 WO 2015/034125

### SUMMARY OF INVENTION

As a characteristic of the inventions, Patent Literature 3 to Patent Literature 5 describe using a bicarbazole derivative as a hole-transporting host material, whereby the performance of phosphorescent devices was significantly improved. When used for applications such as displays, however, even the above-mentioned material system exhibits insufficient performance and there is a demand for further lifetime prolongation.

The object of the present invention is to provide an organic electroluminescence device having a longer lifetime than the lifetimes of conventional ones.

The present invention provides an organic electroluminescence device comprising an anode, a cathode, and an emitting layer between the anode and the cathode, in which the emitting layer comprises a compound represented by the formula (1) and a compound represented by the formula (2):

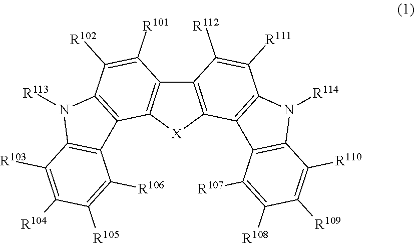

(wherein

R¹⁰¹ to R¹¹² are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 carbon atoms that form a ring (hereinafter referred to as “ring carbon atoms”), a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 atoms that form a ring (hereinafter referred to as “ring atoms”), a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, a carbonyl group substituted by one or more groups selected from the group consisting of alkyl groups including 1 to 25 carbon atoms and aryl groups including 6 to 24 ring carbon atoms, or —P(═O)R¹²⁰R¹²¹,

R¹¹³ and R¹¹⁴ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms,

adjacent R¹⁰¹ to R¹⁴ may be bonded with each other to form a ring,

X is an oxygen atom, a sulfur atom, C(R¹¹⁵)(R¹¹⁶), N(R¹¹⁷), or Si(R¹¹⁸)(R¹¹⁹), and R¹¹⁵ to R¹²¹ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms); and

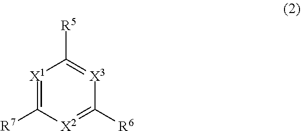

(wherein

X¹ to X³ are independently CR¹¹ or N,

R⁵ to R⁷ and R¹¹ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, a carbonyl group substituted by one or more groups selected from the group consisting of alkyl groups including 1 to 25 carbon atoms and aryl groups including 6 to 24 ring carbon atoms, a cyano group, or —P(═O)R¹⁴R¹⁵

R¹⁴ and R¹⁵ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms, and

when X¹ to X³ are CR¹¹, they may be bonded with any one of adjacent R⁵ to R⁷ to form a ring), and

an electronic apparatus equipped with the above organic electroluminescence device.

The present invention provides an organic electroluminescence device having a lifetime longer than the lifetimes of conventional ones.

### DESCRIPTION OF EMBODIMENTS

A. Organic Electroluminescence Device

The organic electroluminescence device (“electroluminescence” may hereinafter be abbreviated as “EL”) according to one aspect of the present invention is characterized in that it is an organic electroluminescence device comprising an anode, a cathode, and at least an emitting layer between the anode and the cathode, in which the emitting layer comprises a compound represented by the formula (1) and a compound represented by the formula (2).

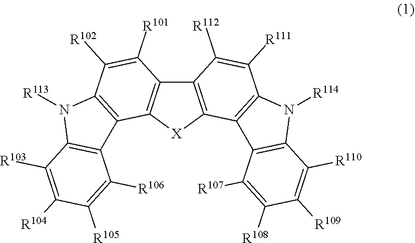

(In the formula (1),

R¹⁰¹ to R¹¹² are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, a carbonyl group substituted by one or more groups selected from the group consisting of alkyl groups including 1 to 25 carbon atoms and aryl groups including 6 to 24 ring carbon atoms, or —P(═O)R¹²⁰R¹²¹.

R¹¹³ and R¹¹⁴ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms.

Adjacent R¹⁰¹ to R¹¹⁴ may be bonded with each other to form a ring.

X is an oxygen atom, a sulfur atom, C(R¹¹⁵)(R¹¹⁶), N(R¹¹⁷), or Si(R¹¹⁸)(R¹¹⁹).

R¹¹⁵ to R¹²¹ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms.)

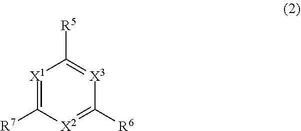

(In the formula (2),

X¹ to X³ are independently CR¹¹ or N. Preferably one or more, more preferably two or more, and still more preferably three of X¹ to X³ are N.

R⁵ to R⁷ and R¹ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, a carbonyl group substituted by one or more groups selected from the group consisting of alkyl groups including 1 to 25 carbon atoms and aryl groups including 6 to 24 ring carbon atoms, a cyano group, or —P(═O)R¹⁴R¹⁵.

R¹⁴ and R¹⁵ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms.

When X¹ to X³ are CR¹¹, they may be bonded with any one of adjacent R⁵ to R⁷ to form a ring.)

The organic EL device according to one aspect of the present invention is characterized by using a combination of compounds represented by the formulae (1) and (2) in an emitting layer.

Patent Literature 1 discloses using a compound represented by the formula (1) as a phosphorescent host material, which is used as a single host material. Patent Literature 1 neither describes nor suggests using the host material in combination with another host material. Moreover, Patent Literature 1 does not examine the lifetime of an device achieved by using the host material in combination with another host material.

The present inventors found that the lifetime of an device can be prolonged with the use of a combination of a compound represented by the formula (1) and a compound represented by the formula (2) as a host material of an emitting layer.

Compounds represented by the formula (1) have a structure represented by the formula below. Due to the electron-donating property (donor property) of the structure, the compounds are supposed to have improved hole-injecting properties.

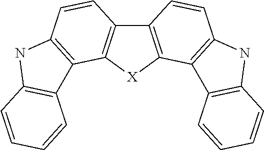

Compounds represented by the formula (2) preferably have any one of the 6-membered heteroaromatic ring structures including 1 to 3 nitrogen atoms as ring atoms represented by the formulae below. Due to the strong electron-accepting properties (acceptor properties) of the structures, the compounds are supposed to have improved electron-injecting properties. As an electron-transporting host material, pyrimidine or triazine having a higher electron-accepting property is particularly preferred.

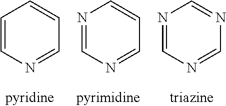

The compounds represented by the formula (1) are materials having higher hole-injecting properties than those of the compounds represented by the formula (2), and have ionization potentials of 5.0 eV to 6.5 eV and preferably 5.3 eV to 5.8 eV, for example. The electron-injecting properties of the compounds represented by the formula (1) are preferably lower than those of the compounds represented by the formula (2), and the affinity (Af) values of the compounds represented by the formula (1) are preferably lower than 2.1 eV. In order to have affinity values lower than 2.1 eV, the compounds represented by the formula (1) preferably do not have a nitrogen-containing 6-membered ring structure (e.g., pyrimidine or triazine) that is electron deficient (namely that has a strong electron-accepting property).

The compounds represented by the formula (2) are materials having higher electron-injecting properties than those of the compounds represented by the formula (1) and preferably have affinity (Af) values of 2.1 eV to 2.6 eV and more preferably 2.2 eV to 2.5 eV, for example.

Ionization potential (Ip) can be measured with a photoelectron spectrometer (AC-3 produced by Riken Keiki Co., Ltd.) in the atmosphere. Specifically, it is measured by irradiating an objective compound with light and by measuring the quantity of electrons generated by charge separation during the irradiation.

Affinity (Af) values can be measured with reference to the description in Forrest et al, Org. El. 2005, 6, 11 to 20.

Effects of prolonging device lifetimes are obtained by using, in a single emitting layer, the compound represented by the formula (1) having a high electron-donating property (donor property) compared with that of a conventional host material a carbazole compound in combination with the compound represented by the formula (2) having a nitrogen-containing 6-membered ring structure that exhibits high luminous efficiency as a phosphorescent host and has a high electron-accepting property (acceptor property).

A single emitting layer comprising two types of host materials can be obtained by a method of co-vapor deposition with the use of two types of vapor deposition sources, a method of vapor deposition with the use of previously mixed materials, or the like.

In the present specification, hydrogen atoms encompass isotopes having different neutron numbers, namely protium, deuterium, and tritium.

In the present specification, the ring carbon number means the number of carbon atoms among atoms constituting a ring per se of a compound having a structure in which atoms are bonded with each other in a ring form (such as a monocyclic compound, a condensed ring compound, a crosslinked compound, a carbocyclic compound, or a heterocyclic compound). When the ring has a substituent, carbon atoms contained in the substituent are not included in the ring carbon number. The same applies to the “ring carbon number” described below unless otherwise specified herein. For example, a benzene ring, a naphthalene ring, a pyridinyl group, and a furanyl group have 6, 10, 5, and 4 ring carbon atoms, respectively. When a benzene ring or a naphthalene ring has for example an alkyl group as a substituent, the carbon number of the alkyl group is not included in the ring carbon number. In addition, when a fluorene ring is bonded with a fluorene ring (including a spirofluorene ring) as a substituent, for example, the carbon number of the substituent fluorene ring is not included in the ring carbon number.

In the present specification, the ring atom number means the number of atoms constituting a ring per se of a compound having a structure in which atoms are bonded with each other in a ring form (such as a monocycle, a condensed ring, or a ring assembly) (such as a monocyclic compound, a condensed ring compound, a crosslinked compound, a carbocyclic compound, or a heterocyclic compound). Neither atoms that do not constitute the ring nor atoms that are included in a substituent when the ring has the substituent are included in the ring atom number. The same applies to the “ring atom number” described below unless otherwise specified herein. For example, a pyridine ring, a quinazoline ring, and a furan ring have 6, 10, and 5 ring atoms, respectively. Neither hydrogen atoms binding to carbon atoms of a pyridine ring or a quinazoline ring nor atoms constituting a substituent are included in the ring atom number. In addition, when a fluorene ring is bonded with a fluorene ring (including a spirofluorene ring) as a substituent, for example, the atom number of the substituent fluorene ring is not included in the ring atom number.

In the present specification, the description “XX to YY carbon atoms” in the expression “substituted or unsubstituted ZZ group including XX to YY carbon atoms” means the number of carbon atoms when the ZZ group is unsubstituted. When the ZZ group has a substituent, the number of carbon atoms contained in the substituent is not included.

In the present specification, the description “XX to YY atoms” in the expression “substituted or unsubstituted ZZ group including XX to YY atoms” means the number of atoms when the ZZ group is unsubstituted. When the ZZ group has a substituent, the number of atoms contained in the substituent is not included.

The term “unsubstituted” in the description “substituted or unsubstituted” means that there is no substitution by the substituent and a hydrogen atom is bonded.

Specific examples of each of the groups in the formulae described above and below and substituents intended in the above description “substituted or unsubstituted” are described.

Examples of the alkyl group include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, and a 1,2,3-trinitropropyl group.

The alkyl group is preferably selected from a methyl group, an ethyl group, a propyl group, an isopropyl group, a n-butyl group, a s-butyl group, an isobutyl group, a t-butyl group, a n-pentyl group, and a n-hexyl group.

The carbon number of the alkyl group is 1 to 50, preferably 1 to 25, and more preferably 1 to 10.

The alkenyl group is a group having a double bond in the above-described alkyl group and has 2 to 50, preferably 2 to 25, and more preferably 2 to 10 carbon atoms. The preferred alkenyl group is a vinyl group.

The alkynyl group is a group having a triple bond in the above-described alkyl group and has 2 to 50, preferably 2 to 25, and more preferably 2 to 10 carbon atoms. The preferred alkynyl group is an ethynyl group.

Examples of the cycloalkyl group include a cyclopropyl group, a cyclobutyl group, a cyclopentyl group, a cyclohexyl group, a 4-methylcyclohexyl group, a 1-adamantyl group, a 2-adamantyl group, a 1-norbornyl group, and 2-norbornyl group.

The ring carbon number of the cycloalkyl group is 3 to 50, preferably 3 to 25, more preferably 3 to 10, still more preferably 3 to 8, and particularly preferably 3 to 6.

The alkoxy group is a group represented by —OY¹⁰ and Y¹⁰ is exemplified by the same groups as the above-described examples of the alkyl group and cycloalkyl group. The carbon number of the alkoxy group is preferably 1 to 25 and more preferably 1 to 10.

The alkylthio group is a group represented by —SY¹⁰ and Y¹⁰ is exemplified by the same groups as the above-described examples of the alkyl group and cycloalkyl group. The carbon number of the alkylthio group is 1 to 50, preferably 1 to 25, and more preferably 1 to 10.

Examples of the halogen atom include fluorine, chlorine, bromine, and iodine atoms and a fluorine atom is preferred.

Examples of the aryl group include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenantrhyl group, a 1-pyrenyl group, a 2-pyrenyl group, a 4-pyrenyl group, a 6-chrysenyl group, a 1-benzo[c]phenanthryl group, a 2-benzo[c]phenanthryl group, a 3-benzo[c]phenanthryl group, a 4-benzo[c]phenanthryl group, a 5-benzo[c]phenanthryl group, a 6-benzo[c]phenanthryl group, a 1-benzo[g]chrysenyl group, a 2-benzo[g]chrysenyl group, a 3-benzo[g]chrysenyl group, a 4-benzo[g]chrysenyl group, a 5-benzo[g]chrysenyl group, a 6-benzo[g]chrysenyl group, a 7-benzo[g]chrysenyl group, a 8-benzo[g]chrysenyl group, a 9-benzo[g]chrysenyl group, a 10-benzo[g]chrysenyl group, a 11-benzo[g]chrysenyl group, a 12-benzo[g]chrysenyl group, a 13-benzo[g]chrysenyl group, a 14-benzo[g]chrysenyl group, a 1-benzo[a]anthryl group, a 2-benzo[a]anthryl group, a 3-benzo[a]anthryl group, a 4-benzo[a]anthryl group, a 5-benzo[a]anthryl group, a 6-benzo[a]anthryl group, a 7-benzo[a]anthryl group, a 8-benzo[a]anthryl group, a 9-benzo[a]anthryl group, a 10-benzo[a]anthryl group, a 11-benzo[a]anthryl group, a 12-benzo[a]anthryl group, a 13-benzo[a]anthryl group, a 14-benzo[a]anthryl group, a 1-triphenyl group, a 2-triphenyl group, a 1-fluorenyl group, a 2-fluorenyl group, a 3-fluorenyl group, a 4-fluorenyl group, a 9-fluorenyl group, a benzofluorenyl group, a dibenzofluorenyl group, a benzofluoranthenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, and a fluoranthenyl group.

The preferred aryl groups are a phenyl group, a 1-naphthyl group, a 2-naphthyl group, a 1-phenanthryl group, a 2-phenanthryl group, a 3-phenanthryl group, a 4-phenanthryl group, a 9-phenanthryl group, a 1-fluorenyl group, a 2-fluorenyl group, a 3-fluorenyl group, a 4-fluorenyl group, a 5-benzo[c]phenanthryl group, a 4-benzo[a]anthryl group, a 7-benzo[a]anthryl group, a 1-triphenyl group, a 2-triphenyl group and a fluoranthenyl group.

The ring carbon number of the aryl group is 6 to 50, preferably 6 to 24, more preferably 6 to 20, and still more preferably 6 to 18.

The arylene group is a divalent group Y²¹ obtained by further removing one hydrogen atom or substituent from the aryl group described above.

The aralkyl group is represented by —Y¹¹-Y²⁰. Examples of Y¹¹ include divalent groups (alkylene groups or cycloalkylene groups) obtained by further removing one hydrogen atom or substituent from the above-described examples of the alkyl group or the cycloalkyl group. As examples of Y²⁰, the above-described examples of the aryl group are mentioned.

The aryloxy group is represented by —OY²⁰ and examples of Y²⁰ are the same as the above-described examples of the aryl group.

The arylthio group is represented by —SY²⁰ and examples of Y²⁰ are the same as the above-described examples of the aryl group.

The arylcarbonyloxy group is represented by —O—(C═O)—Y²⁰ and Y²⁰ is as described above.

The substituted carbonyl group having a substituent selected from alkyl and aryl groups is represented by —(C═O)—Y¹⁰ or —(C═O)—Y²⁰, and Y¹⁰ and Y²⁰ are as described above.

Examples of the heterocyclic group include heterocyclic groups having no aromaticity and aromatic heterocyclic groups having aromaticity (said heterocyclic group is called a heteroaryl group if being monovalent, and a heteroarylene group if being divalent).

Examples of the heterocyclic groups having no aromaticity include cyclic groups that contain a nitrogen atom, an oxygen atom, or a sulfur atom and include 3 to 50, preferably 3 to 20 ring atoms. Specific examples of the heterocyclic ring having no aromaticity include aziridine, oxirane, thiirane, azetidine, oxetane, trimethylenesulfide, pyrrolidine, tetrahydrofuran, tetrahydrothiophene, piperidine, tetrahydropyran, and tetrahydrothiopyran.

As examples of the heterocyclic group, cyclic groups containing a hetero atom such as a nitrogen atom, an oxygen atom, a sulfur atom, a phosphorus atom, or a sulfur atom are mentioned, and an atom selected from the group consisting of a nitrogen atom, an oxygen atom, and a sulfur atom is preferably contained as a ring atom. As the heterocyclic group, a heteroaryl group having aromaticity is preferred. Examples of the heteroaryl group include a 1-pyrrolyl group, a 2-pyrrolyl group, a 3-pyrrolyl group, a pyrazinyl group, a 2-pyridinyl group, a 3-pyridinyl group, a 4-pyridinyl group, a pyrimidinyl group, a triazinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, an azadibenzofuranyl group, an azadibenzothiophenyl group, a diazadibenzofuranyl group, a diazadibenzothiophenyl group, a naphthobenzofuranyl group, a naphthobenzothiophenyl group, a diazaphenanthryl group, a 1-naphthobenzofuranyl group, a 2-naphthobenzofuranyl group, a 3-naphthobenzofuranyl group, a 4-naphthobenzofuranyl group, a 1-naphthobenzothiophenyl group, a 2-naphthobenzothiophenyl group, a 3-naphthobenzothiophenyl group, a 4-naphthobenzothiophenyl group, a 2-benzofurathiophenyl group, a 3-benzothiophenyl group, a 4-benzothiophenyl group, a 5-benzothiophenyl group, a 6-benzothiophenyl group, a 7-benzothiophenyl group, a 1-carbazolyl group, a 2-carbazolyl group, a 3-carbazolyl group, a 4-carbazolyl group, a 9-carbazolyl group, a 1-benzocarbazolyl group, a 2-benzocarbazolyl group, a 3-benzocarbazolyl group, a 4-benzocarbazolyl group, a 9-benzocarbazolyl group, a quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 2-quinazolinyl group, a 4-quinazolinyl group, a 5-quinazolinyl group, a 6-quinazolinyl group, a 7-quinazolinyl group, a 8-quinazolyl group, a 1-phenanthridinyl group, a 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthrolin-2-yl group, a 1,7-phenanthrolin-3-yl group, a 1,7-phenanthrolin-4-yl group, a 1,7-phenanthrolin-5-yl group, a 1,7-phenanthrolin-6-yl group, a 1,7-phenanthrolin-8-yl group, a 1,7-phenanthrolin-9-yl group, a 1,7-phenanthrolin-10-yl group, a 1,8-phenanthrolin-2-yl group, a 1,8-phenanthrolin-3-yl group, a 1,8-phenanthrolin-4-yl group, a 1,8-phenanthrolin-5-yl group, a 1,8-phenanthrolin-6-yl group, a 1,8-phenanthrolin-7-yl group, a 1,8-phenanthrolin-9-yl group, a 1,8-phenanthrolin-10-yl group, a 1,9-phenanthrolin-2-yl group, a 1,9-phenanthrolin-3-yl group, a 1,9-phenanthrolin-4-yl group, a 1,9-phenanthrolin-5-yl group, a 1,9-phenanthrolin-6-yl group, a 1,9-phenanthrolin-7-yl group, a 1,9-phenanthrolin-8-yl group, a 1,9-phenanthrolin-10-yl group, a 1,10-phenanthrolin-2-yl group, a 1,10-phenanthrolin-3-yl group, a 1,10-phenanthrolin-4-yl group, a 1,10-phenanthrolin-5-yl group, a 2,9-phenanthrolin-1-yl group, a 2,9-phenanthrolin-3-yl group, a 2,9-phenanthrolin-4-yl group, a 2,9-phenanthrolin-5-yl group, a 2,9-phenanthrolin-6-yl group, a 2,9-phenanthrolin-7-yl group, a 2,9-phenanthrolin-8-yl group, a 2,9-phenanthrolin-10-yl group, a 2,8-phenanthrolin-1-yl group, a 2,8-phenanthrolin-3-yl group, a 2,8-phenanthrolin-4-yl group, a 2,8-phenanthrolin-5-yl group, a 2,8-phenanthrolin-6-yl group, a 2,8-phenanthrolin-7-yl group, a 2,8-phenanthrolin-9-yl group, a 2,8-phenanthrolin-10-yl group, a 2,7-phenanthrolin-1-yl group, a 2,7-phenanthrolin-3-yl group, a 2,7-phenanthrolin-4-yl group, a 2,7-phenanthrolin-5-yl group, a 2,7-phenanthrolin-6-yl group, a 2,7-phenanthrolin-8-yl group, a 2,7-phenanthrolin-9-yl group, a 2,7-phenanthrolin-10-yl group, a 1-phenazinyl group, a 2-phenazinyl group, a 1-phenothiadinyl group, a 2-phenothiadinyl group, a 3-phenothiadinyl group, a 4-phenothiadinyl group, a 10-phenothiadinyl group, a 1-phenoxadinyl group, a 2-phenoxadinyl group, a 3-phenoxadinyl group, a 4-phenoxadinyl group, a 10-phenoxadinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 1-benzimidazoly group, a 2-benzimidazolyl group, a 4-benzimidazolyl group, a 5-benzimidazolyl group, a 6-benzimidazolyl group, a 7-benzimidazolyl group, a 2-imidazo[1,2-a]pyridinyl group, a 3-imidazo[1,2-a]pyridinyl group, a 5-imidazo[1,2-a]pyridinyl group, a 6-imidazo[1,2-a]pyridinyl group, a 7-imidazo[1,2-a]pyridinyl group, a 8-imidazo[1,2-a]pyridinyl group, a benzimidazol-2-on-1-yl group, a benzimidazol-2-on-3-yl group, a benzimidazol-2-on-4-yl group, a benzimidazol-2-on-5-yl group, a benzimidazol-2-on-6-yl group, and a benzimidazol-2-on-7-yl group.

Preference is given to a pyrazinyl group, a 2-pyridinyl group, a 3-pyridinyl group, a 4-pyridinyl group, a pyrimidinyl group, a triazinyl group, a quinazolinyl group, a diazadibenzofuranyl group, a diazadibenzothiophenyl group, a carbazolyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, a 1-naphthobenzofuranyl group, a 2-naphthobenzofuranyl group, a 3-naphthobenzofuranyl group, a 4-naphthobenzofuranyl group, a 1-naphthobenzothiophenyl group, a 2-naphthobenzothiophenyl group, a 3-naphthobenzothiophenyl group, a 4-naphthobenzothiophenyl group, a 1-carbazolyl group, a 2-carbazolyl group, a 3-carbazolyl group, a 4-carbazolyl group, a 9-carbazolyl group, a 1-benzocarbazolyl group, a 2-benzocarbazolyl group, a 3-benzocarbazolyl group, a 4-benzocarbazolyl group, or a 9-benzocarbazolyl group.

The ring atom number of the heterocyclic group is 3 to 50, preferably 3 to 30, more preferably 5 to 24, and still more preferably 5 to 18.

The ring atom number of the heteroaryl group is 5 to 50, preferably 5 to 30, more preferably 5 to 24, and still more preferably 5 to 18.

The preferred ring atom of the heteroaryl group other than a carbon atom is a nitrogen atom, an oxygen atom, or a sulfur atom.

The heteroarylene group is a divalent group Y³¹ obtained by further removing one hydrogen atom or substituent from the above-described heteroaryl group.

The mono-substituted amino group having a substituent selected from alkyl and aryl groups is represented by —NH(Y¹⁰) or —NH(Y²⁰), and Y¹⁰ and Y²⁰ are as described above.

The di-substituted amino group having a substituent selected from alkyl and aryl groups is represented by —N(Y¹⁰)₂, —N(Y²⁰)₂, or —N(Y¹⁰)(Y²⁰), and Y¹⁰ and Y²⁰ are as described above. When two Y¹⁰ or Y²⁰ are present, they may be identical with or different from each other.

The mono-substituted silyl group having a substituent selected from alkyl and aryl groups is represented by —SiH₂(Y¹⁰) or —SiH₂(Y²⁰).

The di-substituted silyl group having a substituent selected from alkyl and aryl groups is represented by —SiH(Y¹⁰)₂, —SiH(Y²⁰)₂ or —SiH(Y¹⁰)(Y²⁰).

The tri-substituted silyl group having a substituent selected from alkyl and aryl groups is represented by —Si(Y¹⁰)₃, —Si(Y²⁰)₃, —Si(Y¹⁰)₂(Y²⁰), or —Si(Y¹⁰)(Y²⁰)₂. Y¹⁰ and Y²⁰ are as described above and when a plurality of Y¹⁰ or Y²⁰ are present, they may be identical with or different from each other.

The substituted sulfonyl group having a substituent selected from alkyl and aryl groups is represented by —S(═O)₂—Y¹⁰ or —S(═O)₂—Y²⁰, and Y¹⁰ and Y²⁰ are as described above.

The di-substituted phosphoryl group having a substituent selected from alkyl and aryl groups is represented by —O—P(═O)(Y¹⁰)₂, —O—P(═O)(Y²⁰)₂, or —O—P(═O)(Y¹⁰)(Y²⁰), and Y¹⁰ and Y²⁰ are as described above. When two Y¹⁰ or Y²⁰ are present, they may be identical with or different from each other.

The alkylsulfonyloxy group having an alkyl group is represented by —O—S(═O)₂(Y¹⁰), and Y¹⁰ is as described above.

The arylsulfonyloxy group having a substituent selected from aryl groups is represented by —O—S(═O)₂(Y²⁰), and Y²⁰ is as described above.

As the substituents intended in the description “substituted or unsubstituted” in the present specification, a carboxy group, a hydroxyl group, and an amino group are mentioned in addition to the entirety of the above-described examples of the groups. Among them, the alkyl group, alkoxy group, aryloxy group, arylthio group, halogen atom, cyano group, substituted silyl group, aryl group, and heteroaryl group are preferred and the aryl group, heteroaryl group, and cyano group are more preferred.

Moreover, the above-described substituents may be bonded with each other to form a single ring or a condensed ring. In another embodiment, the substituents do not form a ring. Furthermore, the above-described substituents may have additional substituents.

### Preferred Embodiments of Compounds Represented by Formula (1)

In one embodiment of the present invention, X in the formula (1) is preferably an oxygen atom or a sulfur atom.

In one embodiment of the present invention, R¹¹³ and R¹¹⁴ are independently a group selected from the group consisting of a substituted or unsubstituted phenyl group, a substituted or unsubstituted biphenyl group, a substituted or unsubstituted naphthyl group, a substituted or unsubstituted terphenyl group, a substituted or unsubstituted phenanthryl group, a substituted or unsubstituted fluoranthenyl group, a substituted or unsubstituted fluorenyl group, a substituted or unsubstituted triphenylenyl group, a substituted or unsubstituted dibenzofuranyl group, a substituted or unsubstituted dibenzothiophenyl group, and a substituted or unsubstituted carbazolyl group.

When R¹¹³ and R¹¹⁴ each have a substituent, said substituent is preferably an aryl group including 6 to 24 ring carbon atoms, or a heterocyclic group including 3 to 30 ring atoms.

In one embodiment of the present invention, when R¹¹³ and R¹¹⁴ are a substituted carbazolyl group, it may have any one of the condensed ring structures typified by the following basic structures in which the substituents are bonded with each other to form a ring.

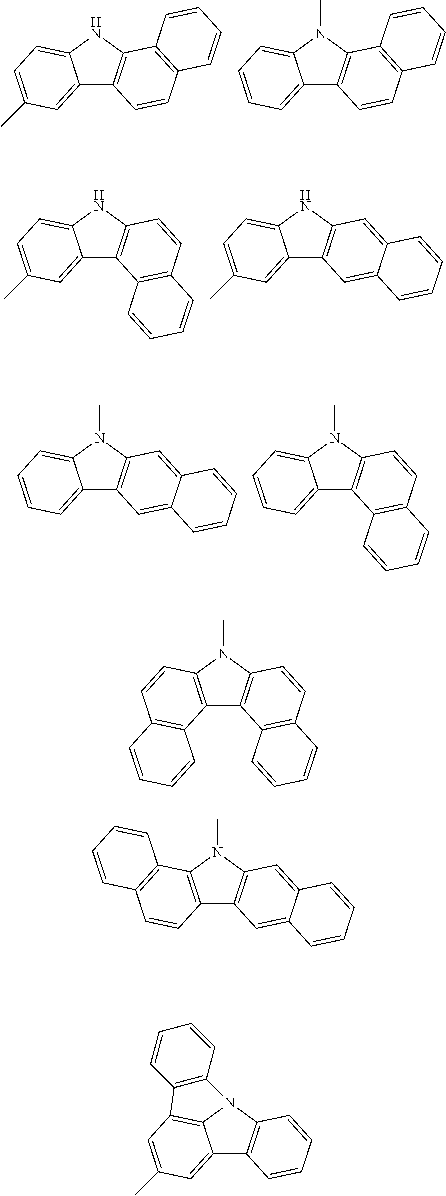

In one embodiment of the present invention, either adjacent two of R¹⁰³ to R¹⁰⁶ or adjacent two of R¹⁰⁷ to R¹¹⁰ or both may be bonded with each other to form a ring.

When a ring is formed, specific examples of the ring include the following partial structures.

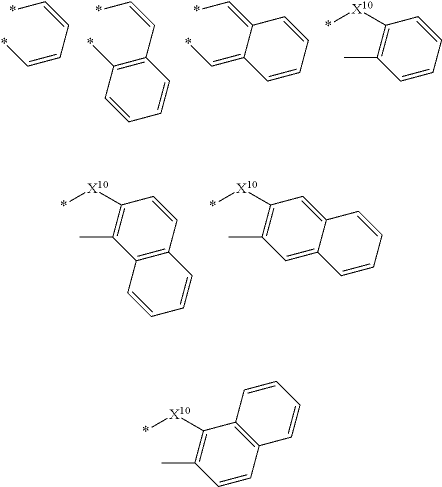

(In the formulae,

X¹⁰ is an oxygen atom, a sulfur atom, or C(R¹³⁰)(R¹³¹).

R¹³⁰ and R¹³¹ are independently a hydrogen atom, a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms.

* is a valence bond to the formula (1) and binds thereto in a position where either adjacent two of R¹⁰³ to R¹⁰⁶ or adjacent two of R¹⁰⁷ to R¹¹⁰, or both are substituted.)

In one embodiment of the present invention, the compounds represented by the formula (1) are preferably compounds represented by the following formula (3).

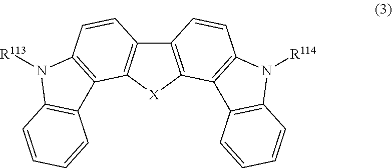

(In the formula (3), R¹¹³, R¹¹⁴, and X are as defined in the formula (1).)

In one embodiment of the present invention, a substituent intended in the description “substituted or unsubstituted” in the formula (1) (for example, substituents when R¹¹³ and R¹¹⁴ are substituted) is preferably selected from the group consisting of a substituted or unsubstituted phenyl group, a substituted or unsubstituted trimethylsilyl group, a substituted or unsubstituted methyl group, a cyano group, a substituted or unsubstituted methoxy group, a substituted or unsubstituted phenoxy group, a fluorine atom, a substituted or unsubstituted carbazolyl group, a substituted or unsubstituted benzofuranyl group, a substituted or unsubstituted benzothiophenyl group, and a substituted or unsubstituted benzimidazolyl group.

The following are specific examples of the compounds represented by the formula (1), but the compounds are not limited thereto.

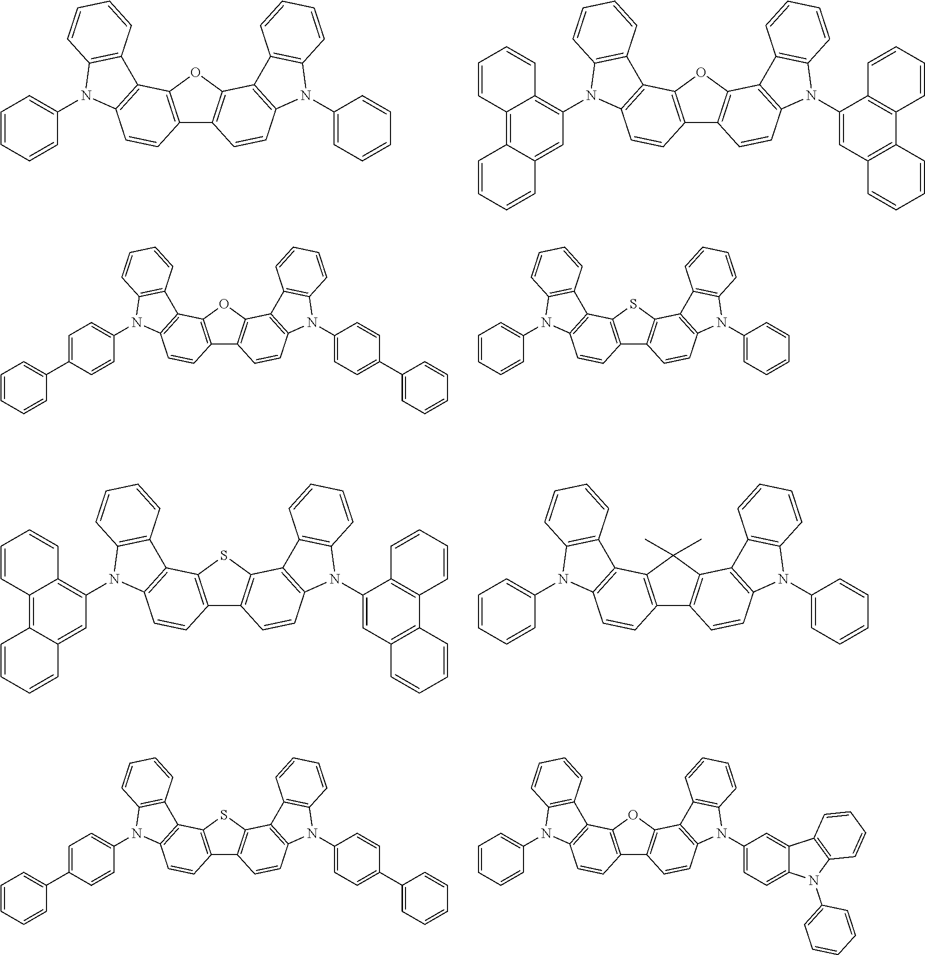

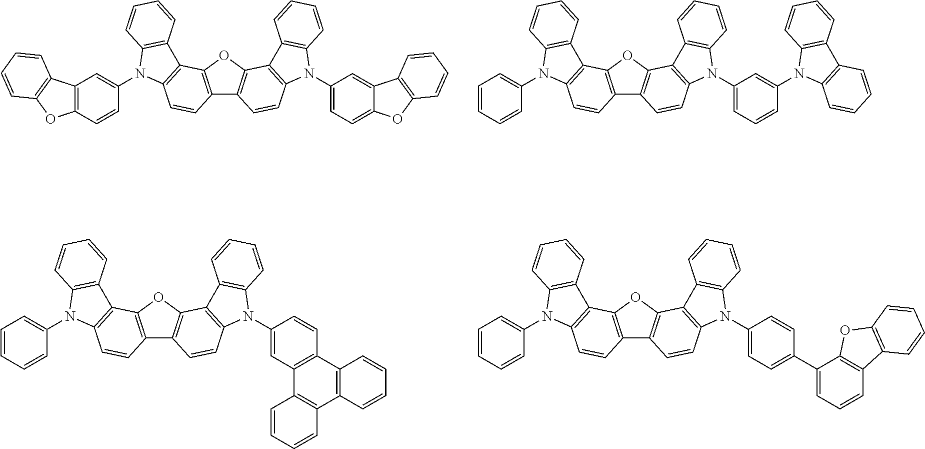

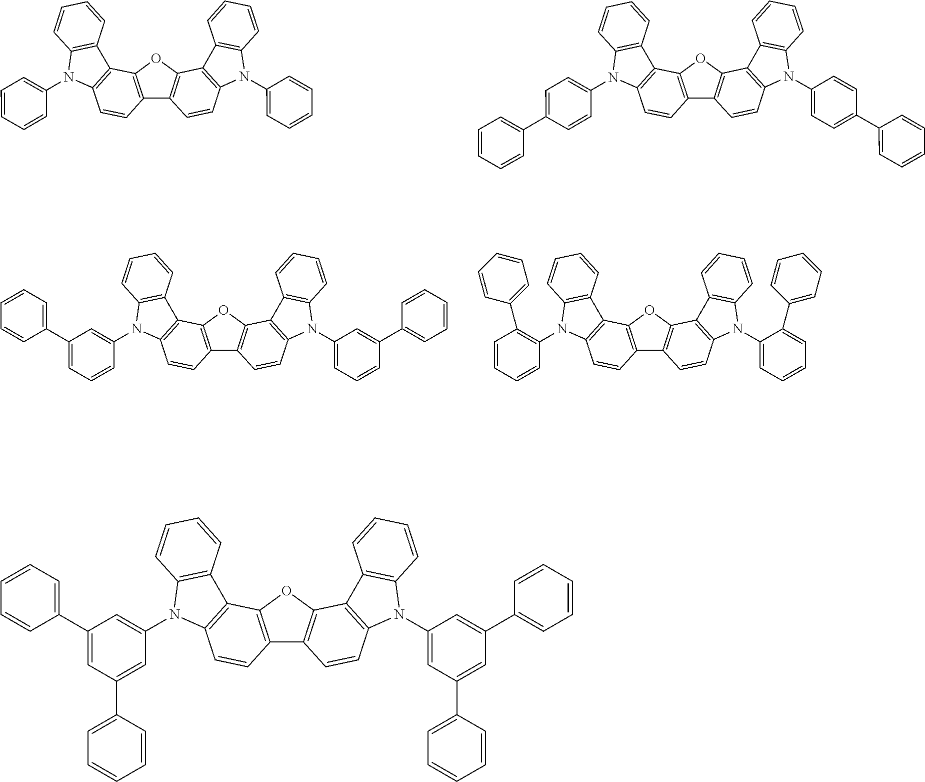 [SMILES: c1ccc(-n2c3ccccc3c3c4oc5c(ccc6c5c5ccccc5n6-c5ccccc5)c4ccc32)cc1 ; c1ccc(-c2ccc(-n3c4ccccc4c4c5oc6c(ccc7c6c6ccccc6n7-c6ccc(-c7ccccc7)cc6)c5ccc43)cc2)cc1 ; c1ccc(-c2cccc(-n3c4ccccc4c4c5oc6c(ccc7c6c6ccccc6n7-c6cccc(-c7ccccc7)c6)c5ccc43)c2)cc1 ; c1ccc(-c2ccccc2-n2c3ccccc3c3c4oc5c(ccc6c5c5ccccc5n6-c5ccccc5-c5ccccc5)c4ccc32)cc1 ; c1ccc(-c2cc(-c3ccccc3)cc(-n3c4ccccc4c4c5oc6c(ccc7c6c6ccccc6n7-c6cc(-c7ccccc7)cc(-c7ccccc7)c6)c5ccc43)c2)cc1]

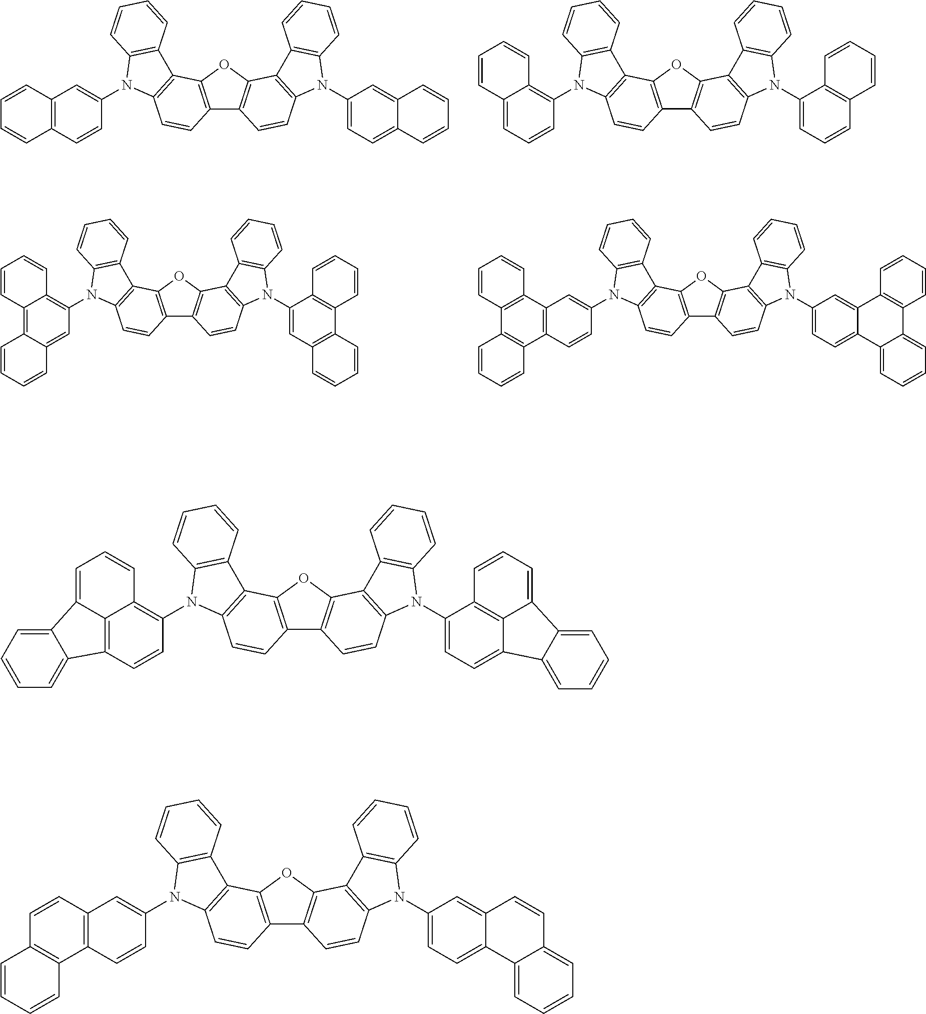

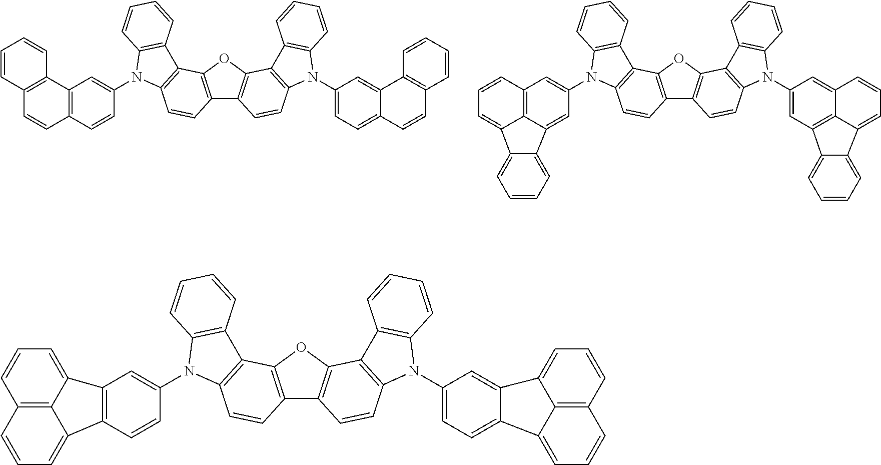

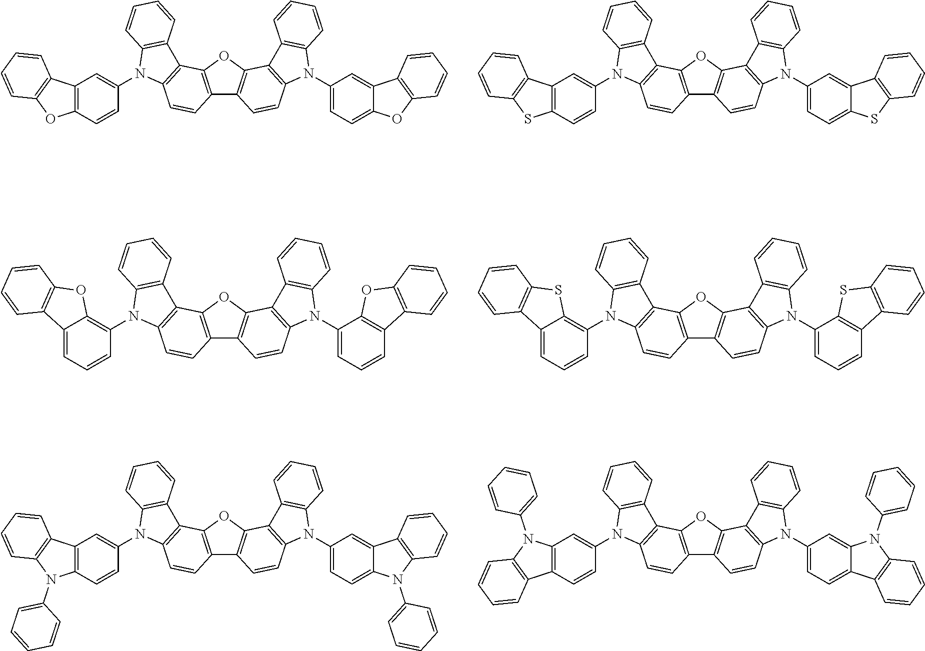

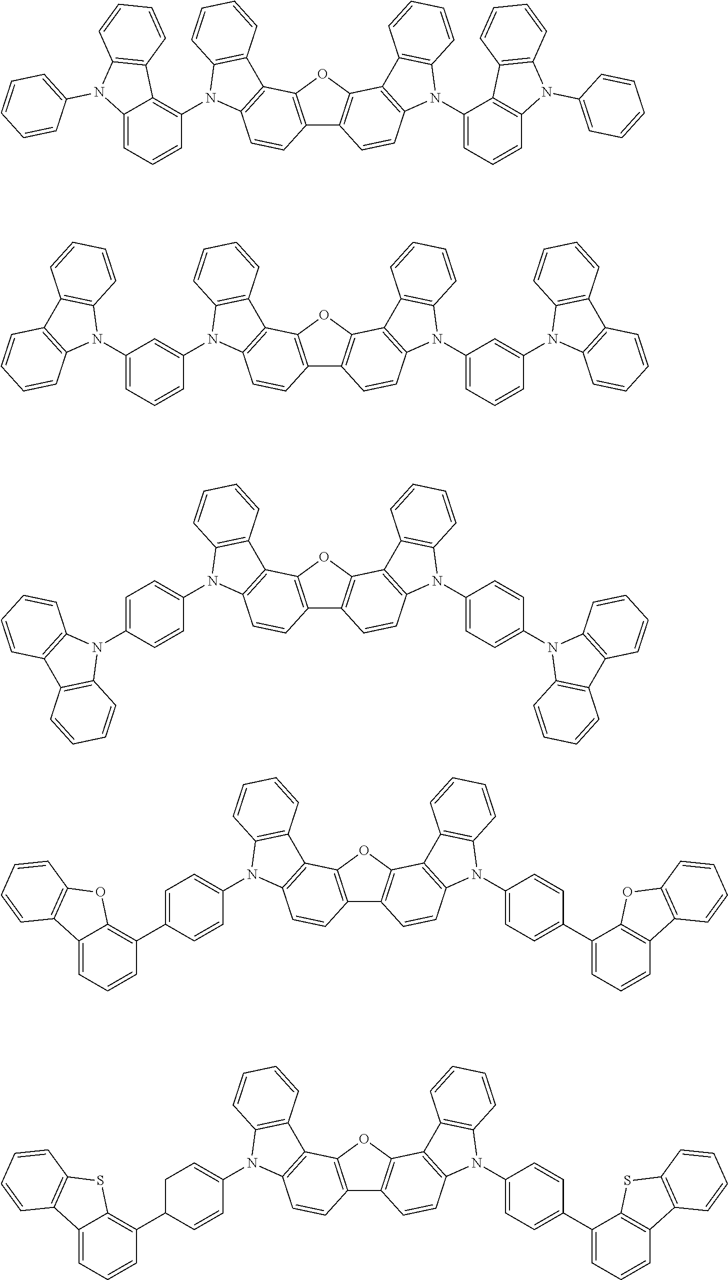

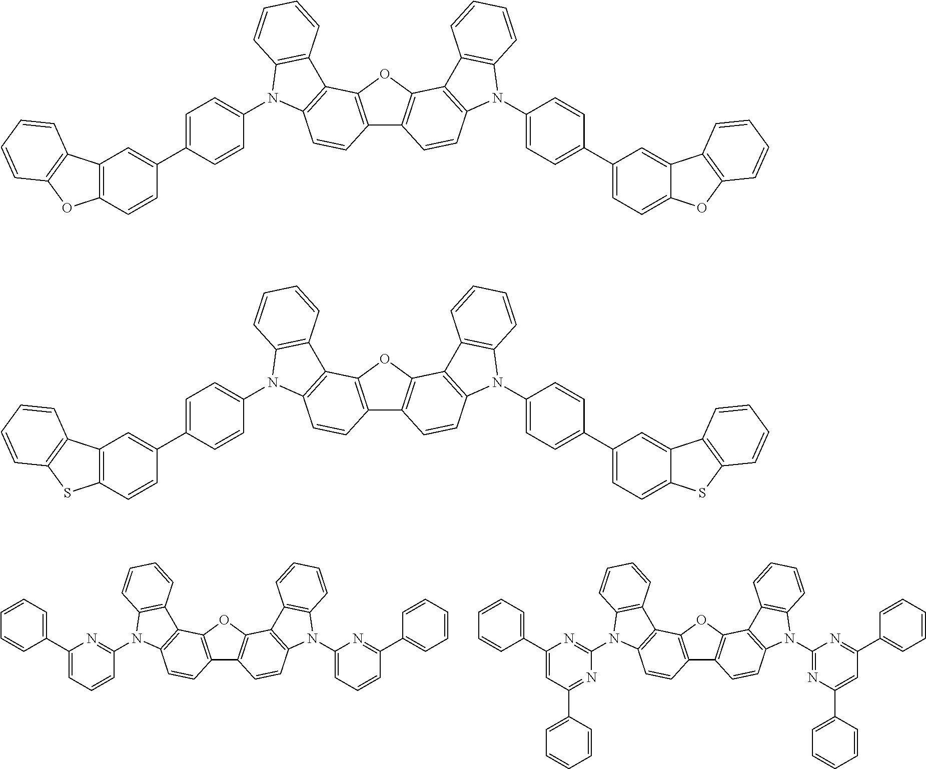

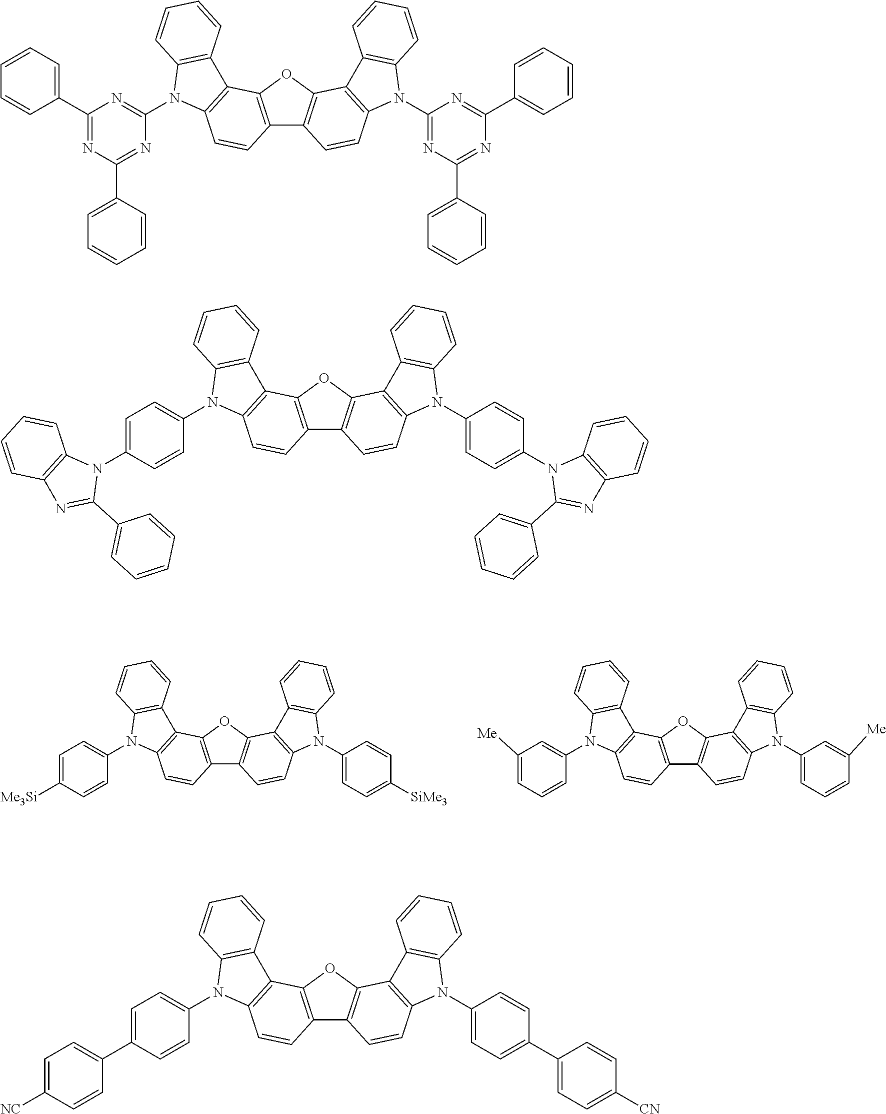 [SMILES: c1ccc(-c2nc(-c3ccccc3)nc(-n3c4ccccc4c4c5oc6c(ccc7c6c6ccccc6n7-c6nc(-c7ccccc7)nc(-c7ccccc7)n6)c5ccc43)n2)cc1 ; c1ccc(-c2nc3ccccc3n2-c2ccc(-n3c4ccccc4c4c5oc6c(ccc7c6c6ccccc6n7-c6ccc(-n7c(-c8ccccc8)nc8ccccc87)cc6)c5ccc43)cc2)cc1 ; C[Si](C)(C)c1ccc(-n2c3ccccc3c3c4oc5c(ccc6c5c5ccccc5n6-c5ccc([Si](C)(C)C)cc5)c4ccc32)cc1 ; Cc1cccc(-n2c3ccccc3c3c4oc5c(ccc6c5c5ccccc5n6-c5cccc(C)c5)c4ccc32)c1 ; N#Cc1ccc(-c2ccc(-n3c4ccccc4c4c5oc6c(ccc7c6c6ccccc6n7-c6ccc(-c7ccc(C#N)cc7)cc6)c5ccc43)cc2)cc1]

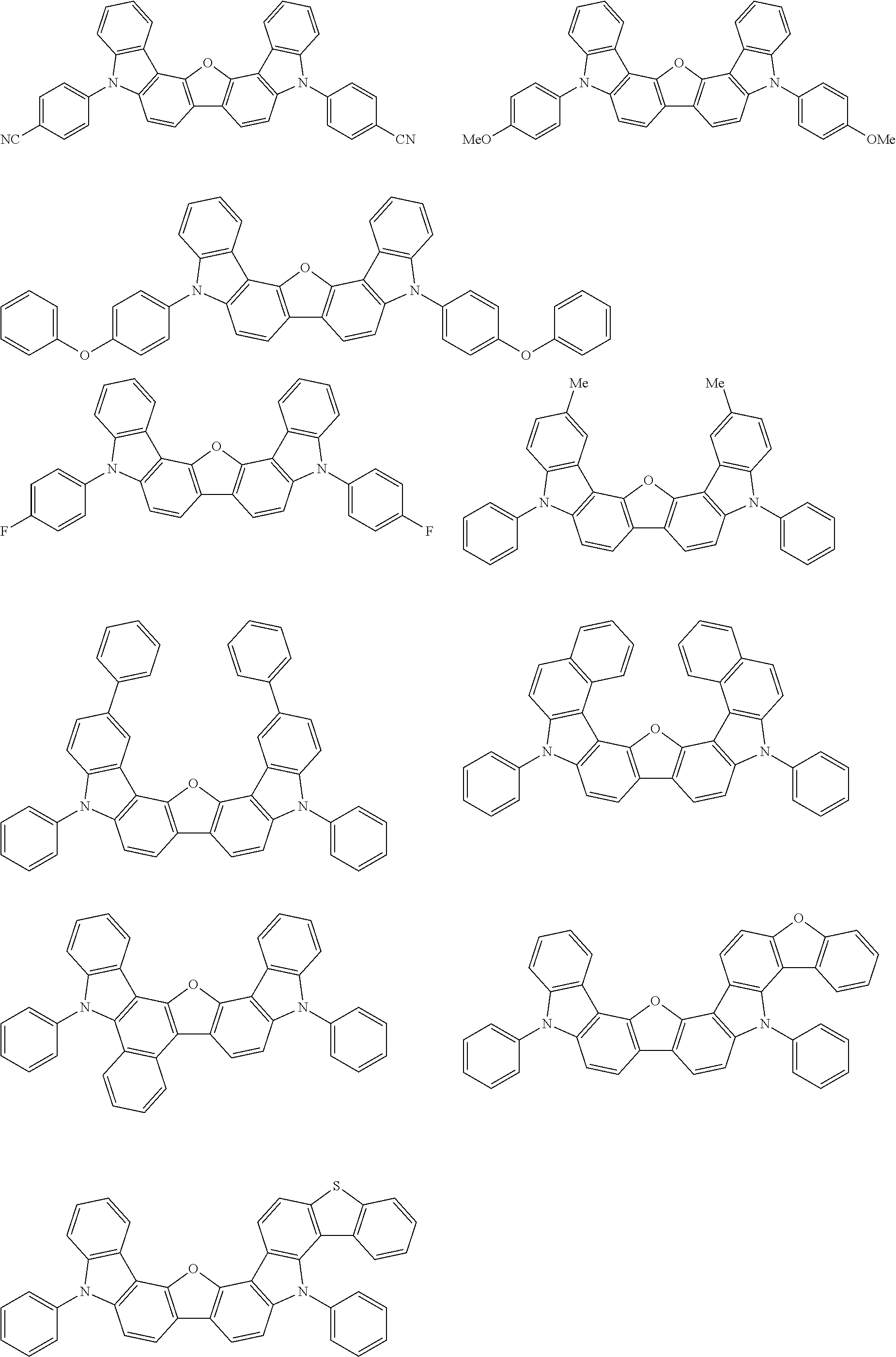

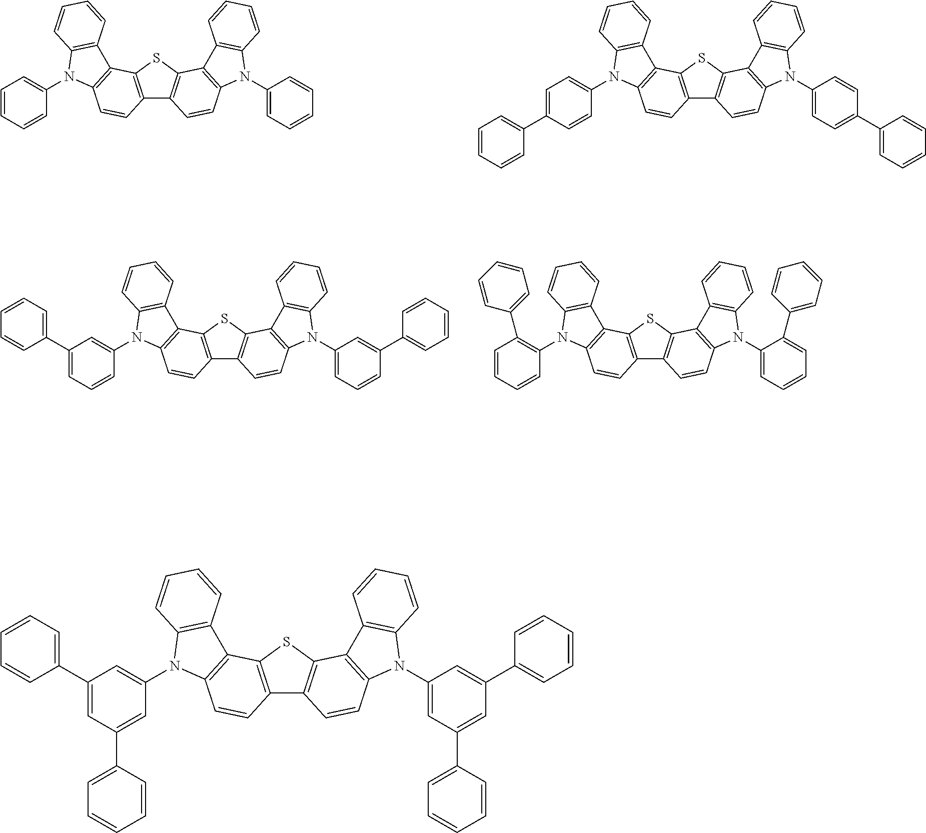 [SMILES: c1ccc(-n2c3ccccc3c3c4sc5c(ccc6c5c5ccccc5n6-c5ccccc5)c4ccc32)cc1 ; c1ccc(-c2ccc(-n3c4ccccc4c4c5sc6c(ccc7c6c6ccccc6n7-c6ccc(-c7ccccc7)cc6)c5ccc43)cc2)cc1 ; c1ccc(-c2cccc(-n3c4ccccc4c4c5sc6c(ccc7c6c6ccccc6n7-c6cccc(-c7ccccc7)c6)c5ccc43)c2)cc1 ; c1ccc(-c2ccccc2-n2c3ccccc3c3c4sc5c(ccc6c5c5ccccc5n6-c5ccccc5-c5ccccc5)c4ccc32)cc1 ; c1ccc(-c2cc(-c3ccccc3)cc(-n3c4ccccc4c4c5sc6c(ccc7c6c6ccccc6n7-c6cc(-c7ccccc7)cc(-c7ccccc7)c6)c5ccc43)c2)cc1]

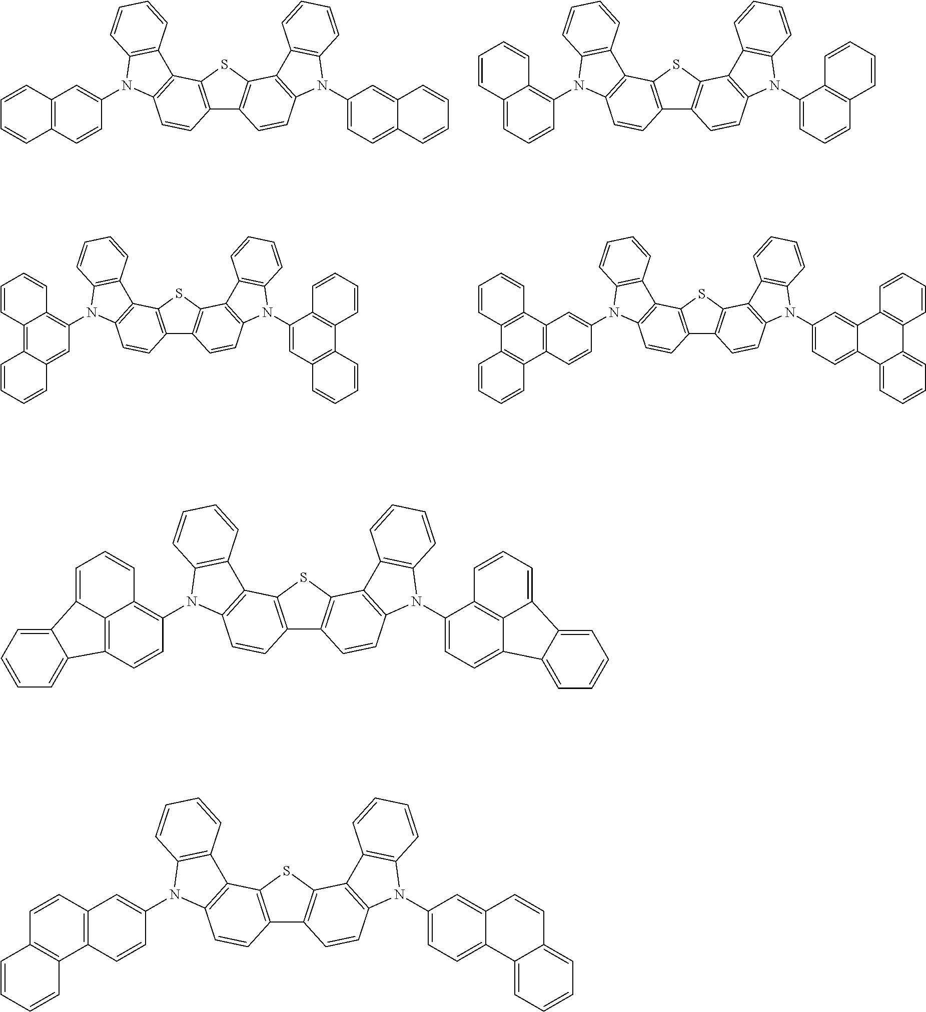

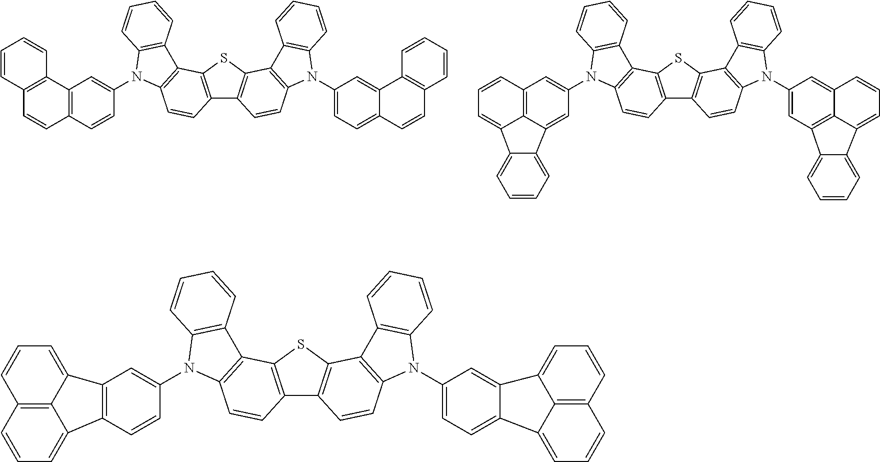

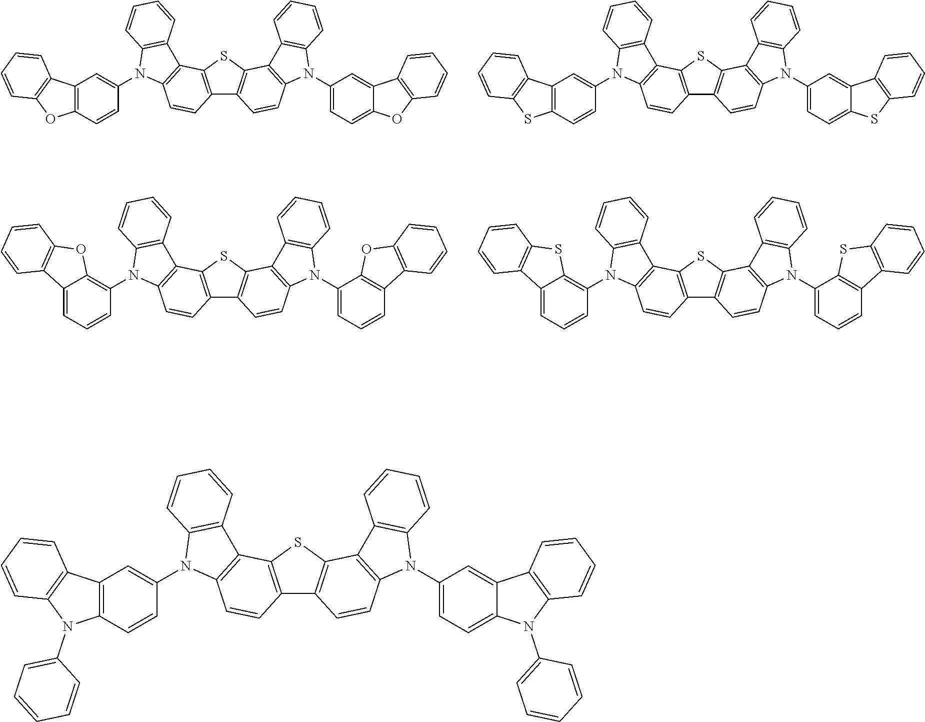

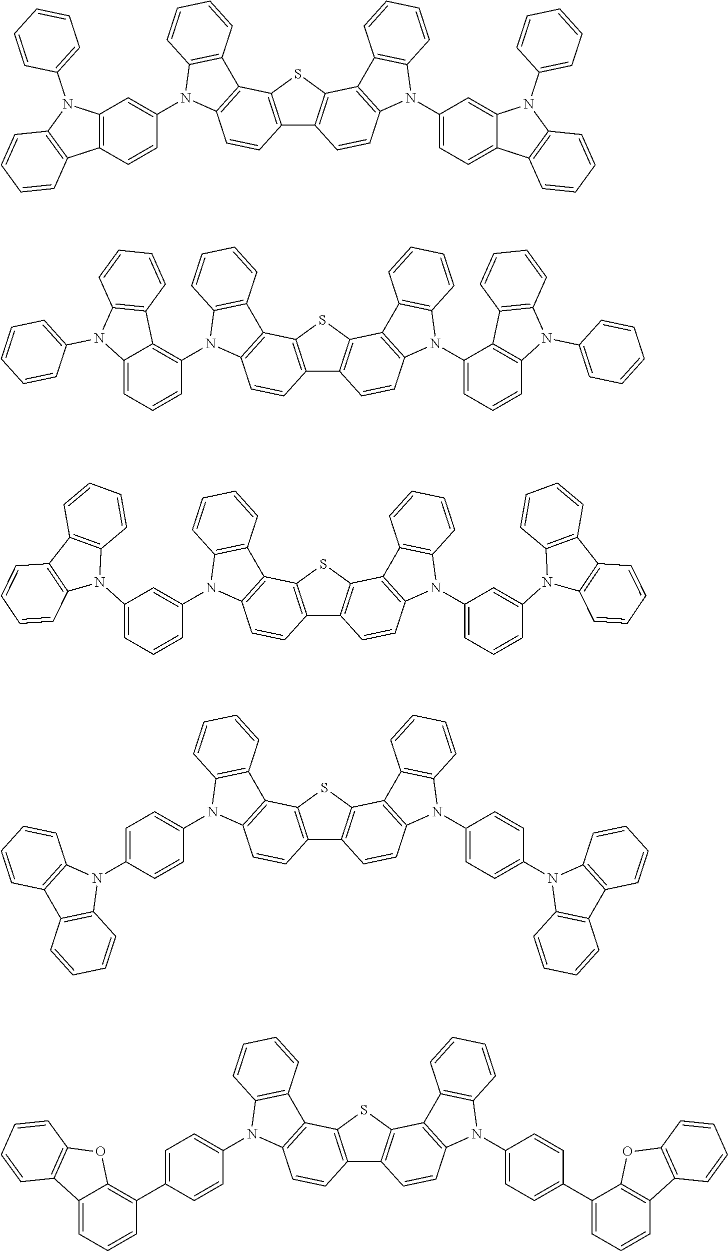

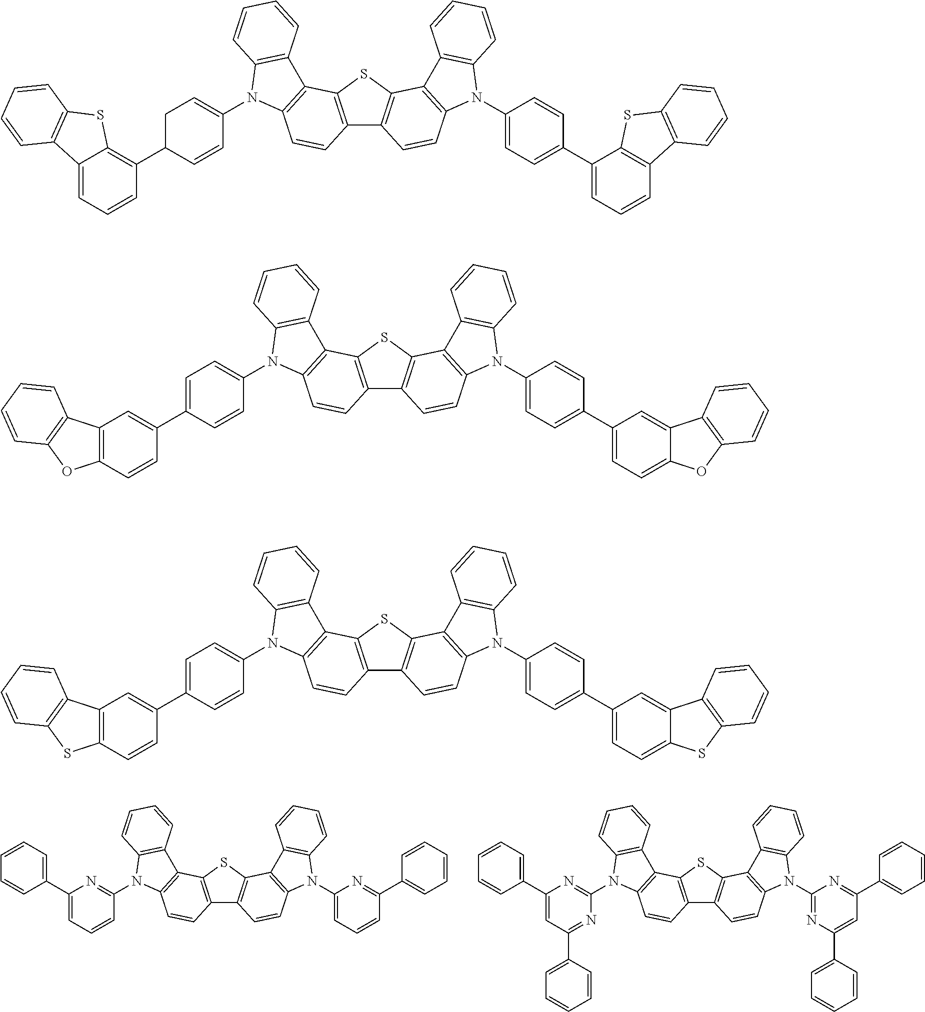

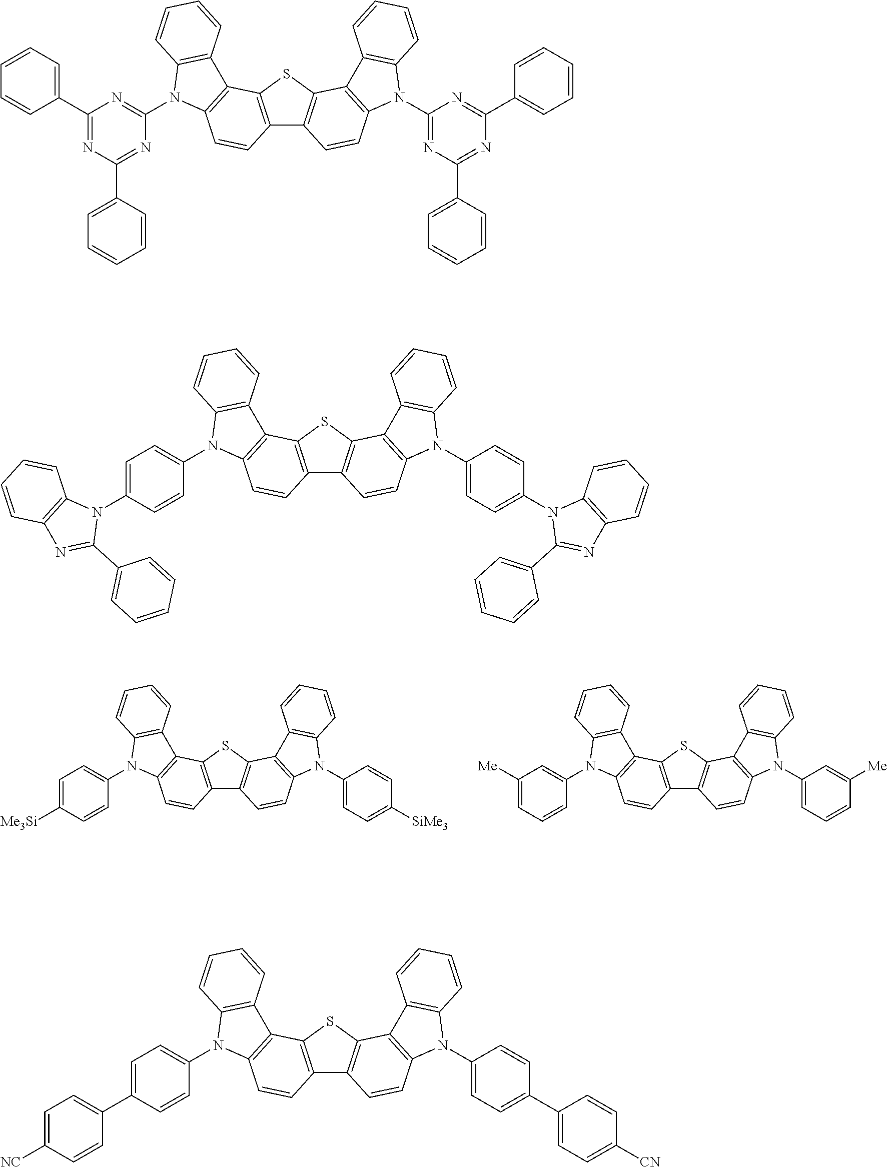 [SMILES: c1ccc(-c2nc(-c3ccccc3)nc(-n3c4ccccc4c4c5sc6c(ccc7c6c6ccccc6n7-c6nc(-c7ccccc7)nc(-c7ccccc7)n6)c5ccc43)n2)cc1 ; c1ccc(-c2nc3ccccc3n2-c2ccc(-n3c4ccccc4c4c5sc6c(ccc7c6c6ccccc6n7-c6ccc(-n7c(-c8ccccc8)nc8ccccc87)cc6)c5ccc43)cc2)cc1 ; C[Si](C)(C)c1ccc(-n2c3ccccc3c3c4sc5c(ccc6c5c5ccccc5n6-c5ccc([Si](C)(C)C)cc5)c4ccc32)cc1 ; Cc1cccc(-n2c3ccccc3c3c4sc5c(ccc6c5c5ccccc5n6-c5cccc(C)c5)c4ccc32)c1 ; N#Cc1ccc(-c2ccc(-n3c4ccccc4c4c5sc6c(ccc7c6c6ccccc6n7-c6ccc(-c7ccc(C#N)cc7)cc6)c5ccc43)cc2)cc1]

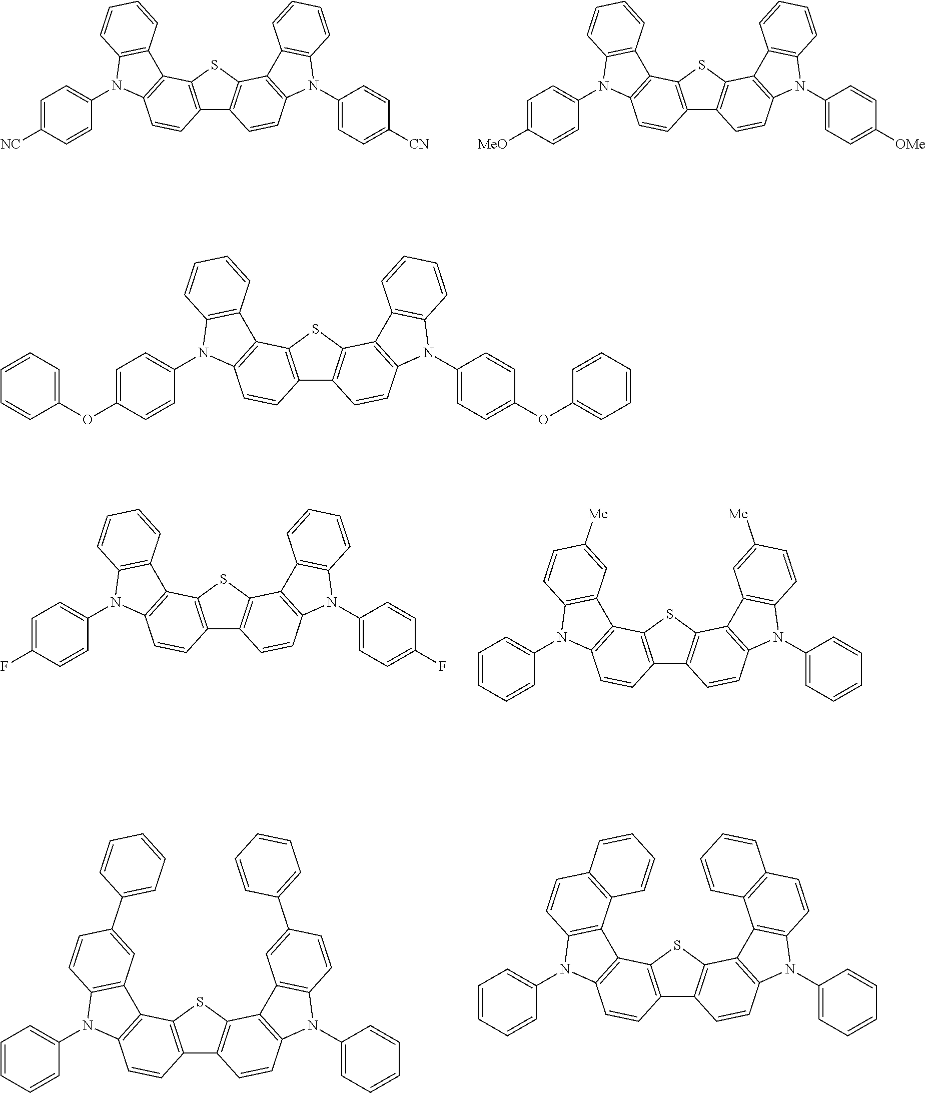

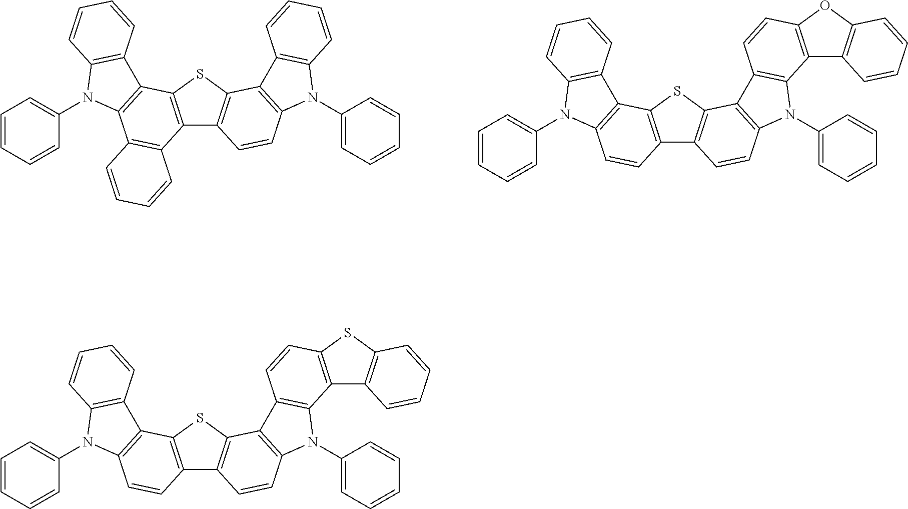 [SMILES: c1ccc(-n2c3ccccc3c3c4sc5c(c6ccccc6c6c5c5ccccc5n6-c5ccccc5)c4ccc32)cc1 ; c1ccc(-n2c3ccccc3c3c4sc5c(ccc6c5c5ccc7oc8ccccc8c7c5n6-c5ccccc5)c4ccc32)cc1 ; c1ccc(-n2c3ccccc3c3c4sc5c(ccc6c5c5ccc7sc8ccccc8c7c5n6-c5ccccc5)c4ccc32)cc1]

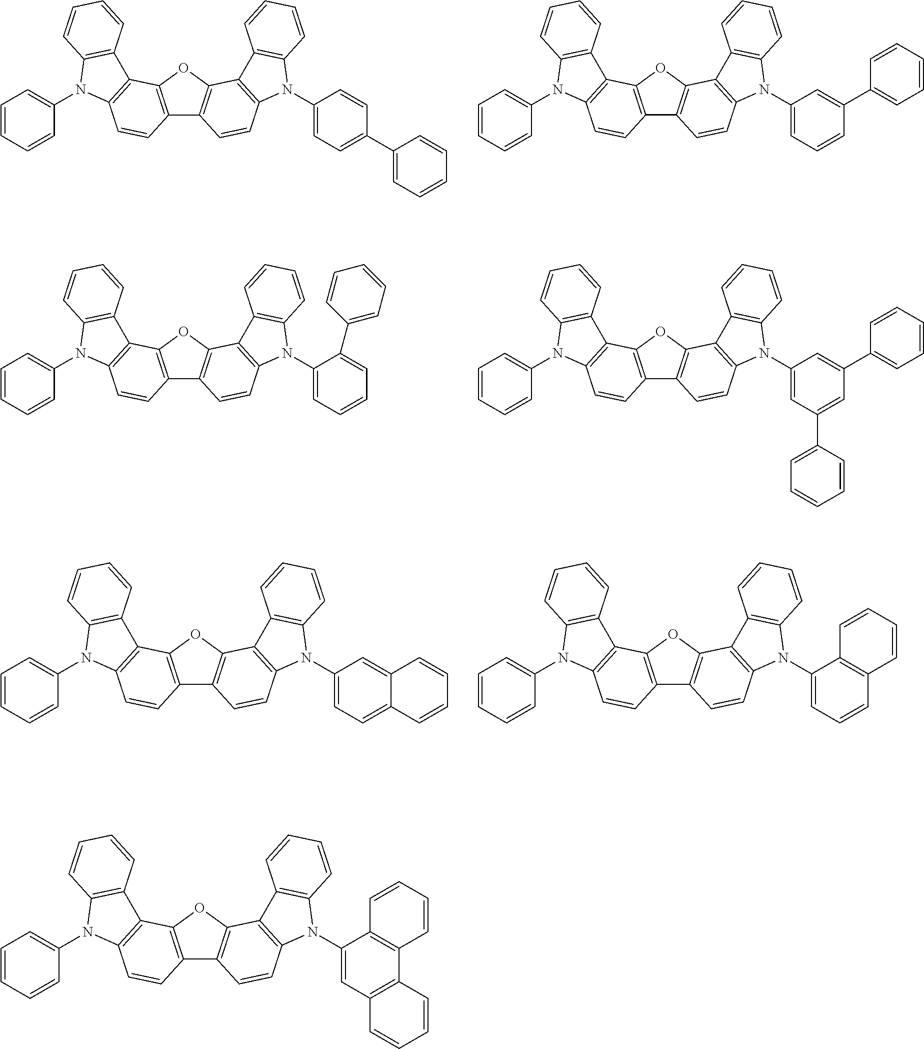

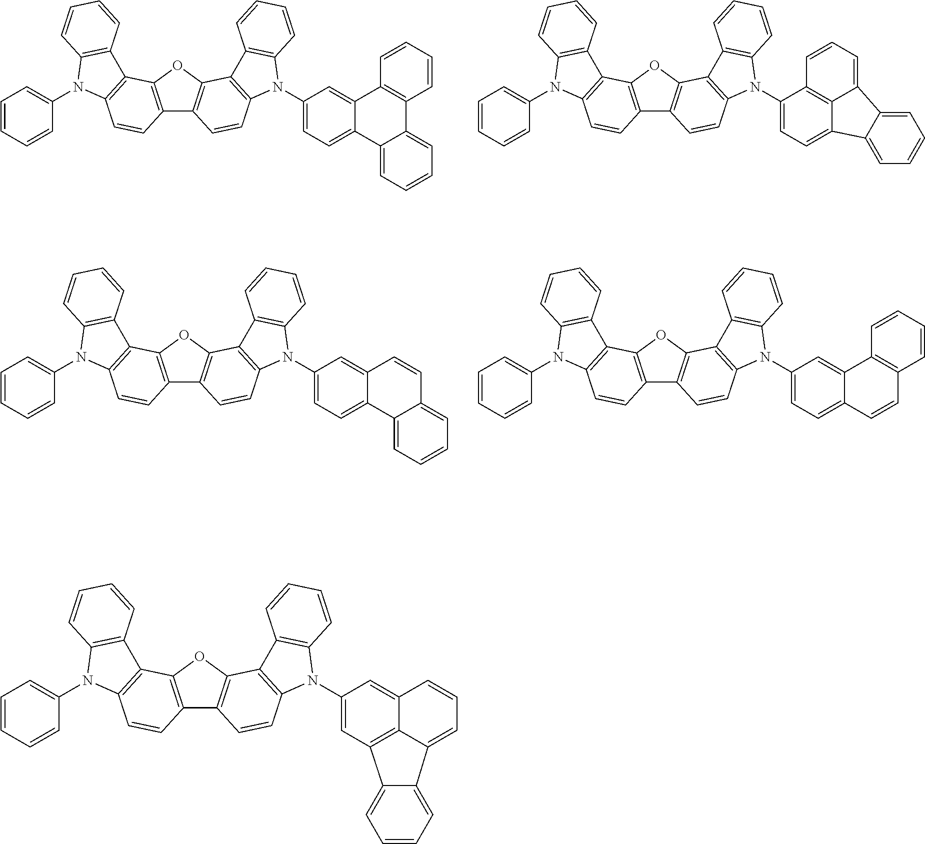

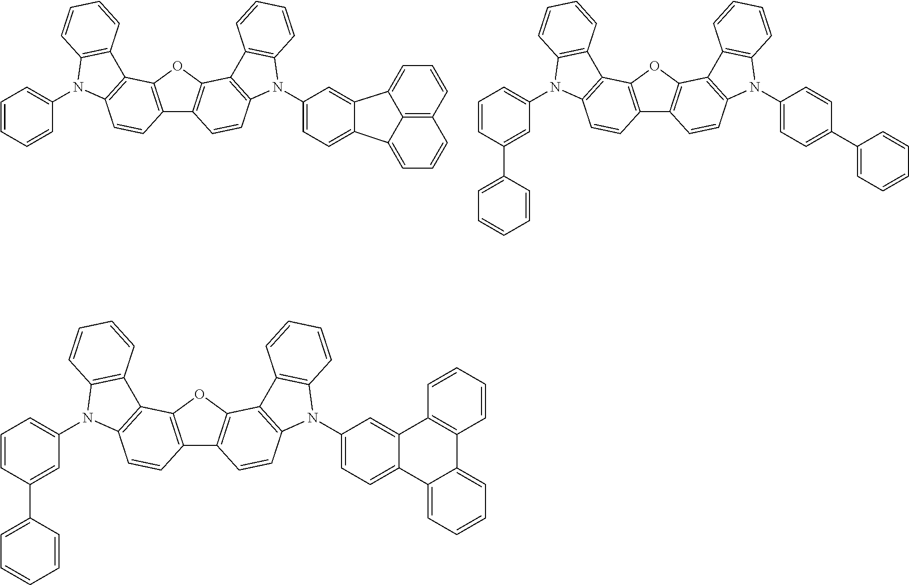

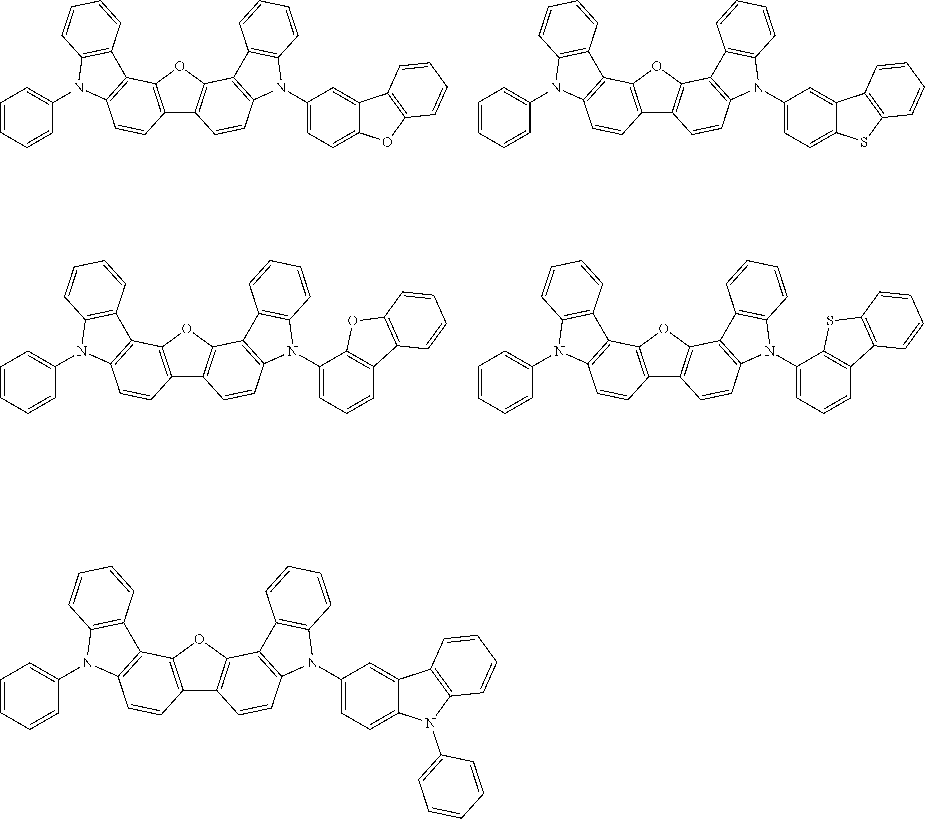

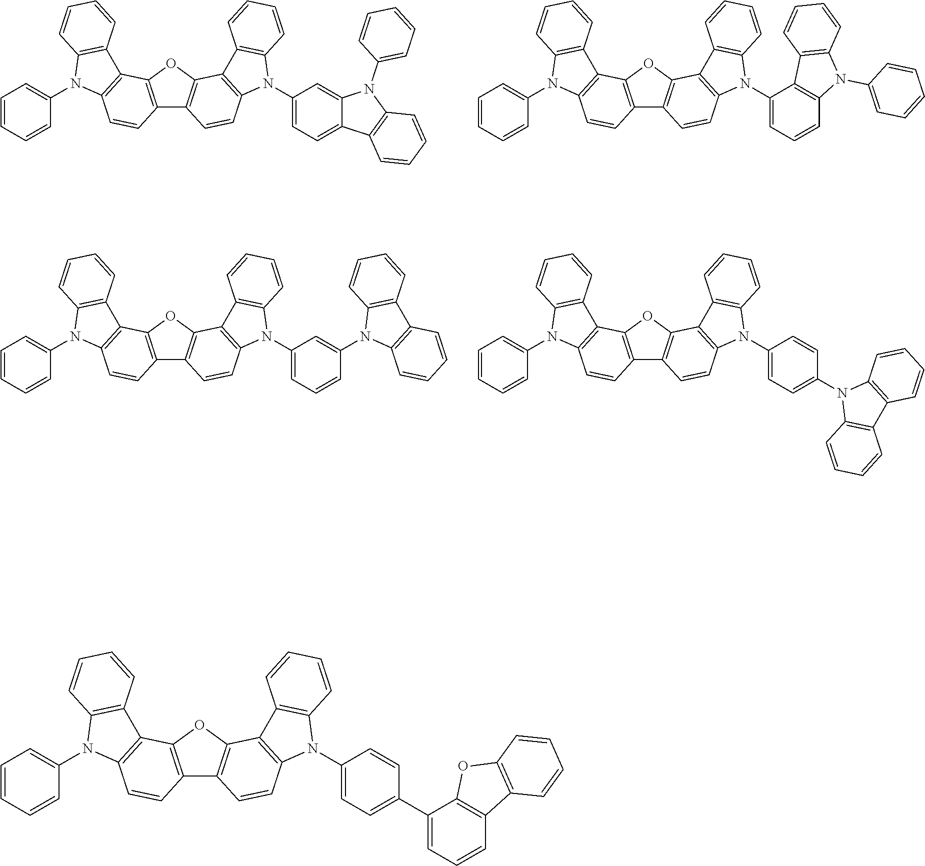

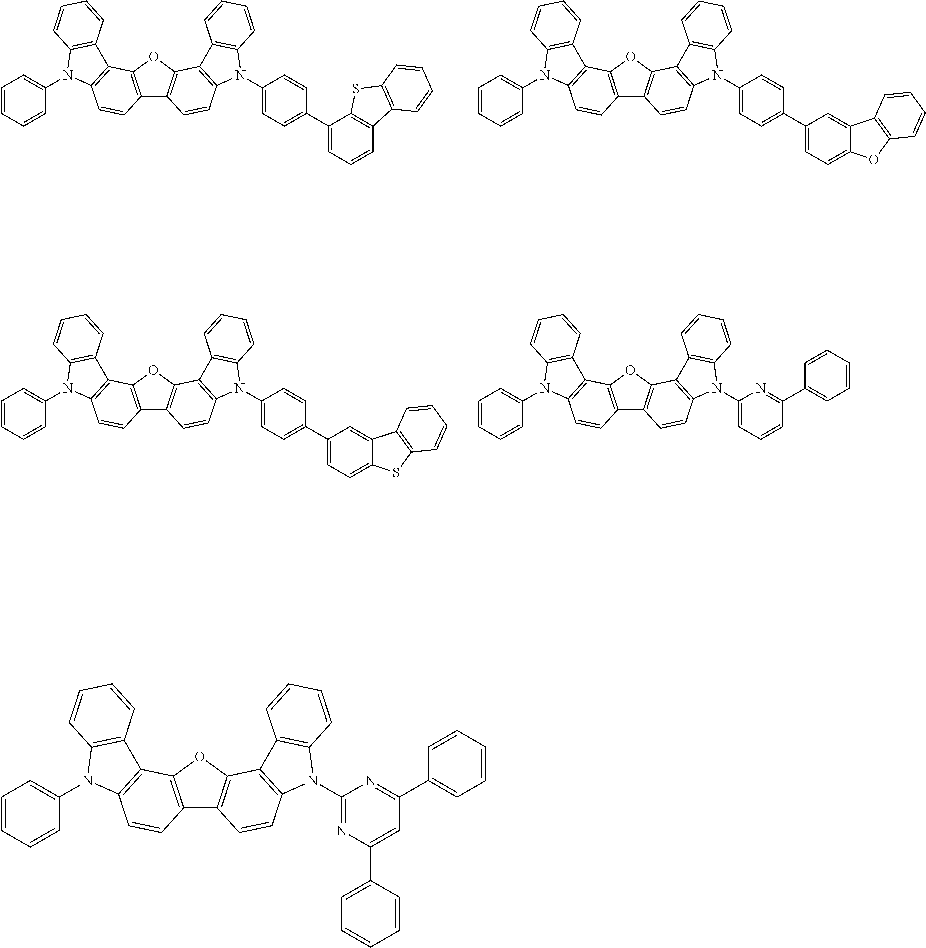

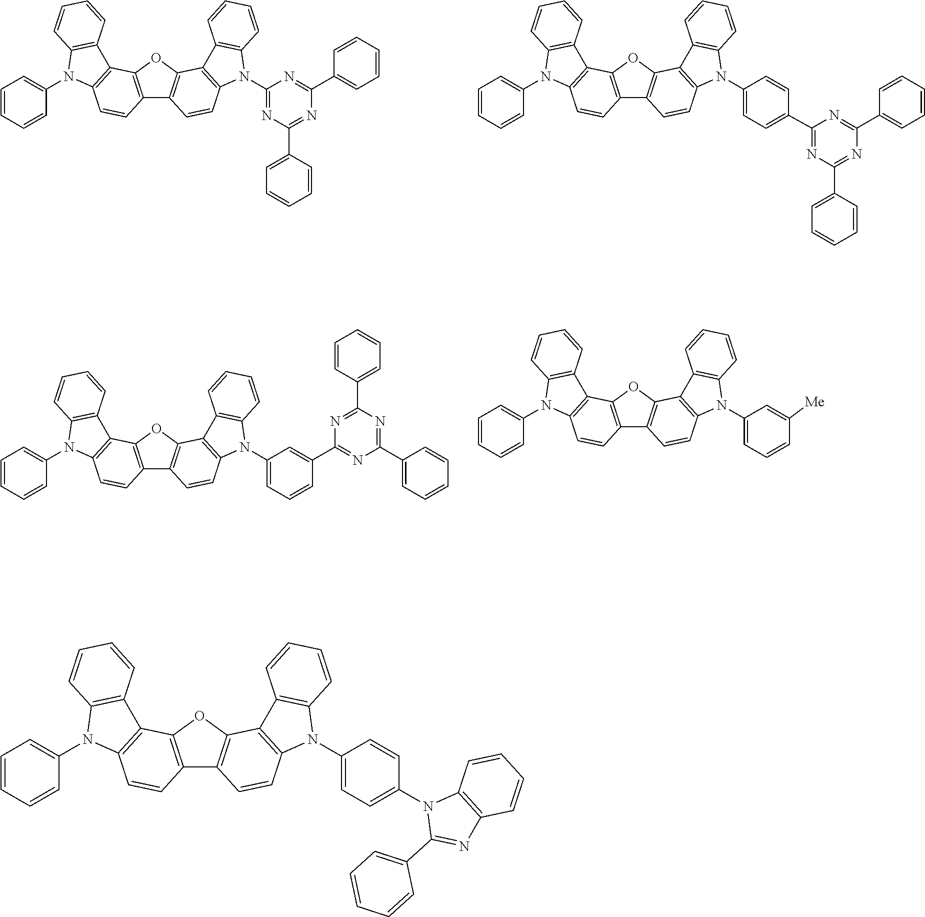

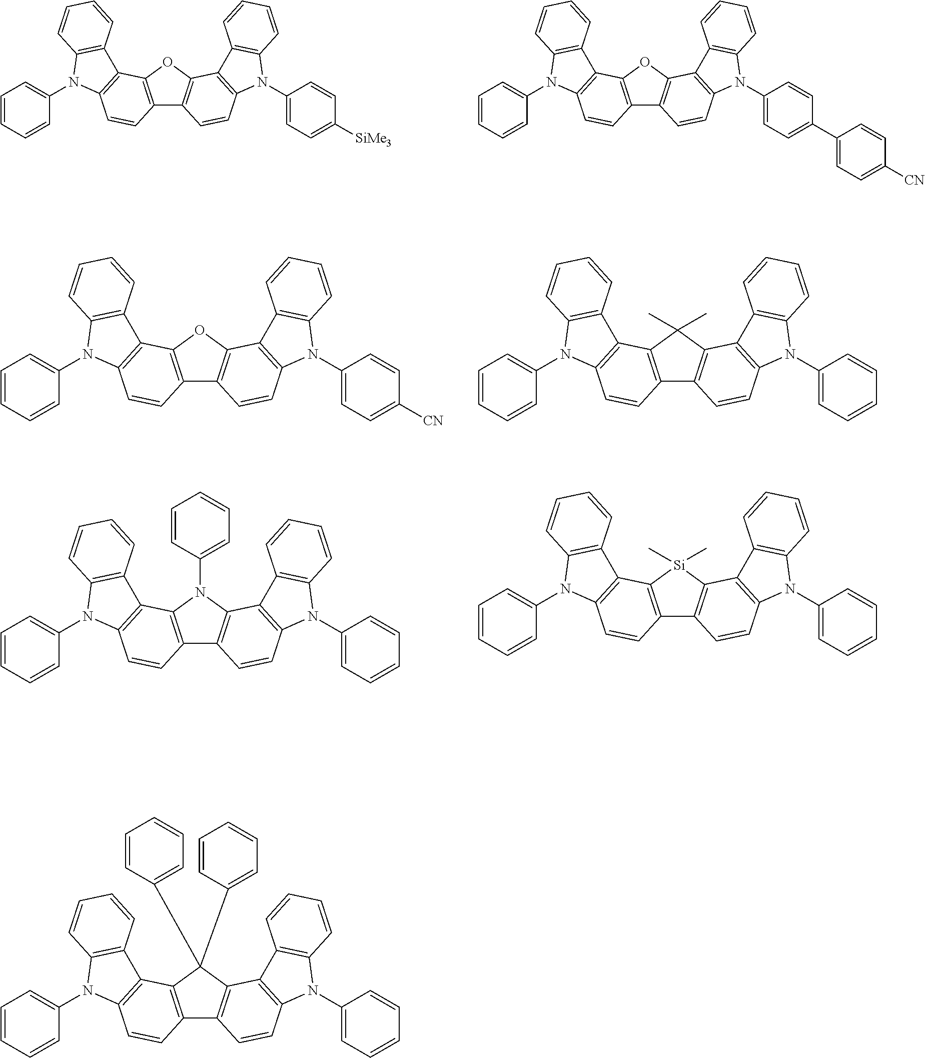

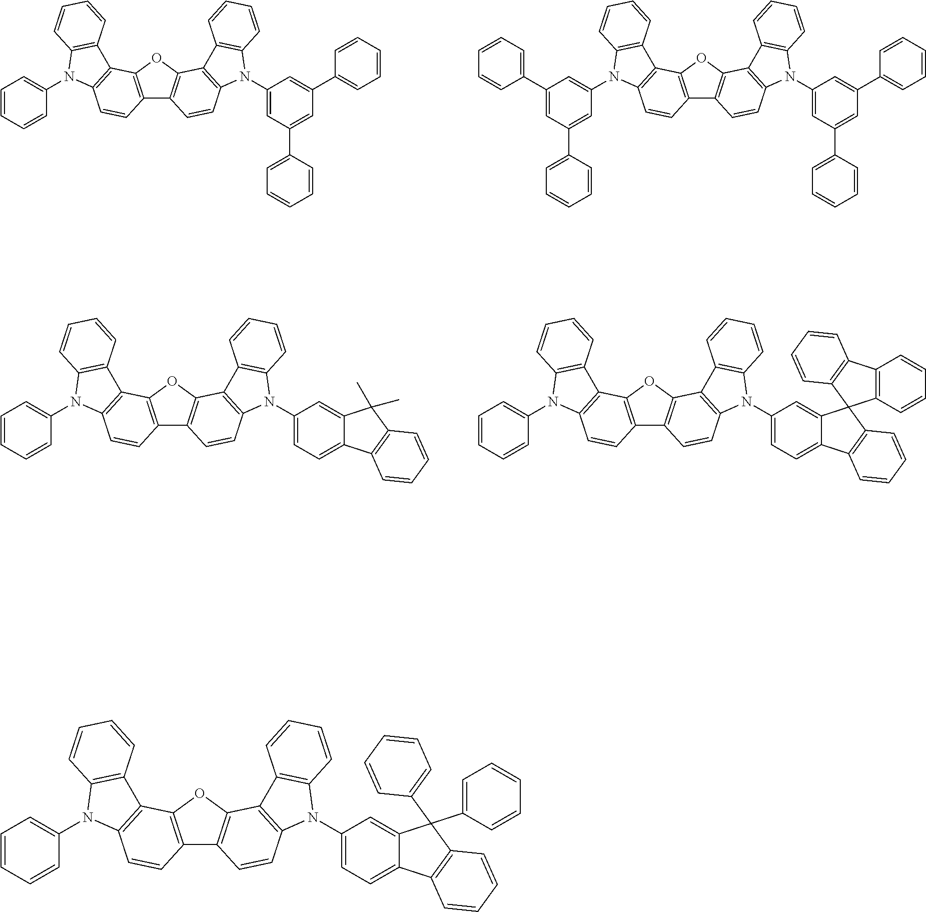 [SMILES: c1ccc(-c2cc(-c3ccccc3)cc(-n3c4ccccc4c4c5oc6c(ccc7c6c6ccccc6n7-c6ccccc6)c5ccc43)c2)cc1 ; c1ccc(-c2cc(-c3ccccc3)cc(-n3c4ccccc4c4c5oc6c(ccc7c6c6ccccc6n7-c6cc(-c7ccccc7)cc(-c7ccccc7)c6)c5ccc43)c2)cc1 ; CC1(C)c2ccccc2-c2ccc(-n3c4ccccc4c4c5oc6c(ccc7c6c6ccccc6n7-c6ccccc6)c5ccc43)cc21 ; c1ccc(-n2c3ccccc3c3c4oc5c(ccc6c5c5ccccc5n6-c5ccc6c(c5)C5(c7ccccc7-c7ccccc75)c5ccccc5-6)c4ccc32)cc1 ; c1ccc(-n2c3ccccc3c3c4oc5c(ccc6c5c5ccccc5n6-c5ccc6c(c5)C(c5ccccc5)(c5ccccc5)c5ccccc5-6)c4ccc32)cc1]

The compound represented by the formula (1) can be synthesized by a known conventional method described in WO 2014/057684A1, for example.

### Preferred Embodiments of Compounds Represented by Formula (2)

In one embodiment of the present invention, the compounds represented by the formula (2) are preferably compounds represented by the formula (21).

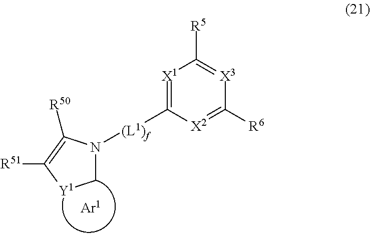

[In the formula (21),

X¹ to X³, R⁵, and R⁶ are as defined in the formula (2).

L¹ is a substituted or unsubstituted arylene group including 6 to 24 ring carbon atoms or a substituted or unsubstituted heteroarylene group including 5 to 30 ring atoms, and is preferably a substituted or unsubstituted arylene group including 6 to 24 ring carbon atoms.

f is an integer of 0 to 4; when f is 2 to 4, a plurality of L¹ may be identical with or different from each other, and when f is 0, the group containing X¹ to X³ is bonded with the group containing Y¹ via a single bond.

Y¹ is a carbon atom or a nitrogen atom.

Ar¹ is a substituted or unsubstituted aromatic hydrocarbon ring including 6 to 24 ring carbon atoms that shares a carbon atom and Y¹ with the adjacent nitrogen-containing 5-membered ring and is condensed to the nitrogen-containing 5-membered ring, or a substituted or unsubstituted aromatic heterocyclic ring including 5 to 30 ring atoms that shares a carbon atom and Y¹ with the adjacent nitrogen-containing 5-membered ring and is condensed to the nitrogen-containing 5-membered ring.

R⁵⁰ and R⁵¹ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, or —P(═O)R¹⁴R¹⁵, or R⁵⁰ and R⁵¹ are bonded with each other to form a substituted or unsubstituted aromatic ring group including 6 to 24 ring carbon atoms.

R¹⁴ and R¹⁵ are as defined in the formula (2).]

The compounds represented by the formula (21) are preferably compounds represented by the formula (22).

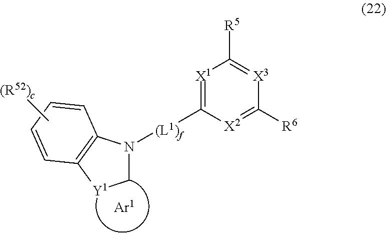

[In the formula (22),

X¹ to X³, R⁵, R⁶, L¹, f, Y¹, and Ar¹ are as defined in the formula (21).

R⁵² is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, or —P(═O)R¹⁴R¹⁵.

R¹⁴ and R¹⁵ are as defined in the formula (2).

c is an integer of 0 to 4; when c is 2 to 4, a plurality of R⁵² may be identical with or different from each other and adjacent R⁵² may be bonded with each other to form a ring.]

The compounds represented by the formula (22) are preferably compounds represented by the formula (23).

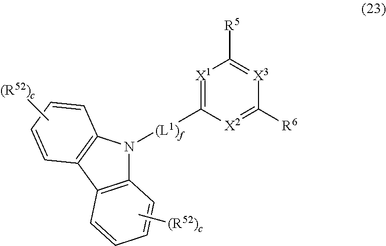

[In the formula (23),

X¹ to X³, R⁵, R⁶, L¹, f, R⁵² and c are as defined in the formula (22).]

The compounds represented by the formula (23) are preferably compounds represented by the formula (24).

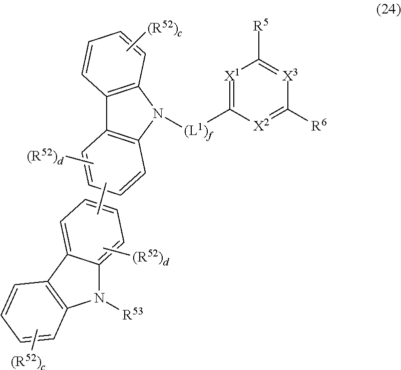

[In the formula (24),

X¹ to X³, R⁵, R⁶, L¹, f, R⁵², and c are as defined in the formula (23).

d is an integer of 0 to 3; when d is 2 or 3, a plurality of R⁵² may be identical with or different from each other.

R⁵³ is a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms.]

In another embodiment, the compounds represented by the formula (22) are preferably compounds represented by the formula (25).

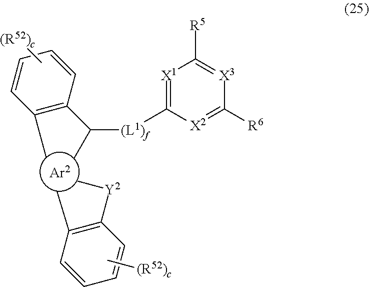

[In the formula (25),

X¹ to X³, R⁵, R⁶, L¹, f, R⁵², and c are as defined in the formula (22).

Y² is CR⁵⁴R⁵⁵, NR⁵⁶, an oxygen atom or a sulfur atom.

R⁵⁴ to R⁵⁶ are a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms.

Ar² is a substituted or unsubstituted aromatic hydrocarbon ring including 6 to 24 ring carbon atoms that shares two carbon atoms of each of adjacent two 5-membered rings and is condensed to the two 5-membered rings, or a substituted or unsubstituted aromatic heterocyclic ring including 5 to 30 ring atoms that shares two carbon atoms of each of adjacent two 5-membered rings and is condensed to the two 5-membered rings.]

The compounds represented by the formula (25) are preferably compounds each represented by any one of the formulae (26A) to (26F).

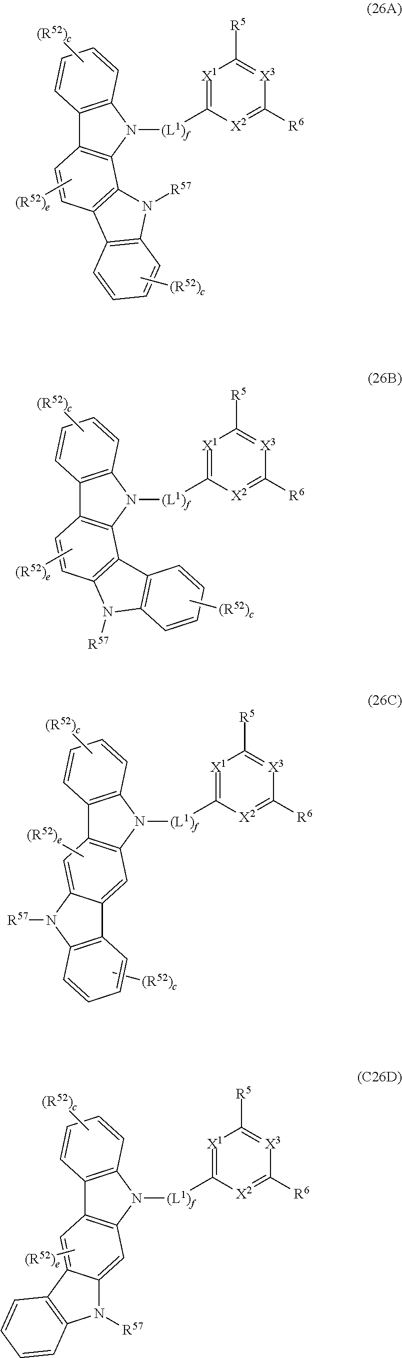

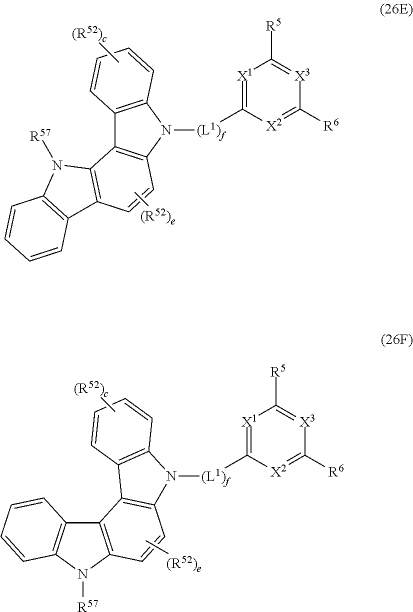

[In the formulae (26A) to (26F),

X¹ to X³, R⁵, R⁶, L¹, f, R⁵², and c are as defined in the formula (25).

R⁵⁷ is a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms.

e is an integer of 0 to 2; when e is 2, a plurality of R⁵² may be identical with or different from each other.]

The compounds represented by the formula (25) are preferably compounds each represented by any one of the formulae (27A) to (27F).

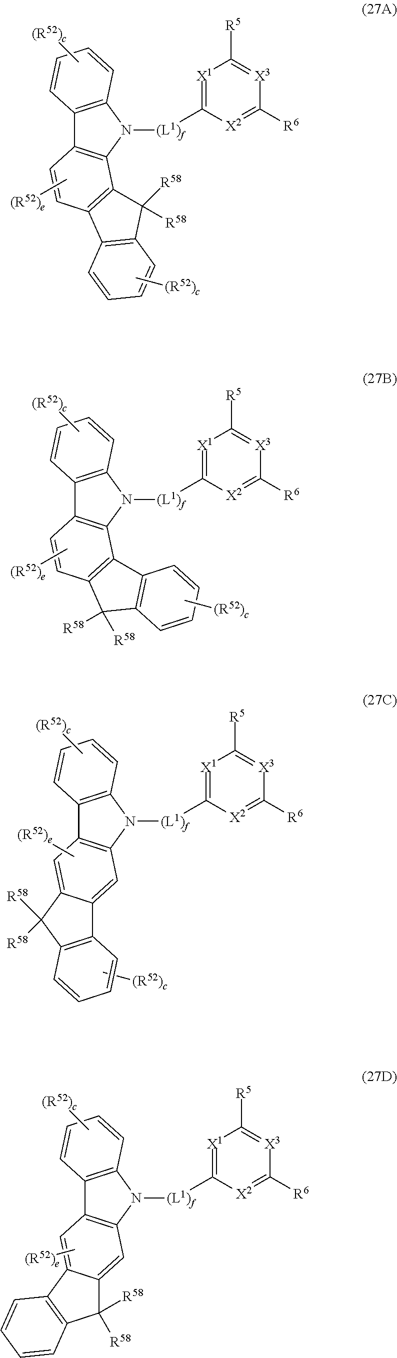

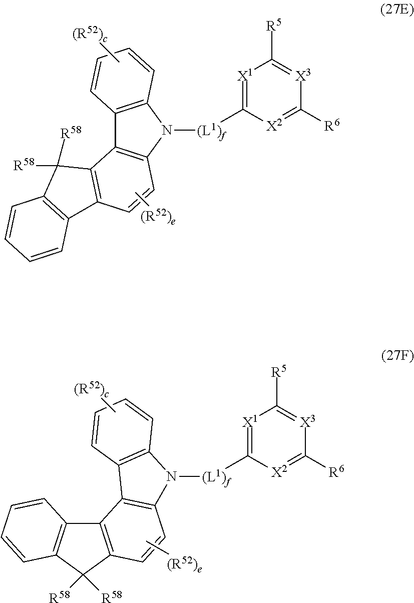

[In the formulae (27A) to (27F),

X¹ to X³, R⁵, R⁶, L¹, f, R⁵², and c are as defined in the formula (25).

R⁵⁸ is a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms.

e is an integer of 0 to 2; when e is 2, a plurality of R⁵² may be identical with or different from each other.]

The compounds represented by the formula (25) are preferably compounds each represented by any one of the formulae (27′A) to (27′F).

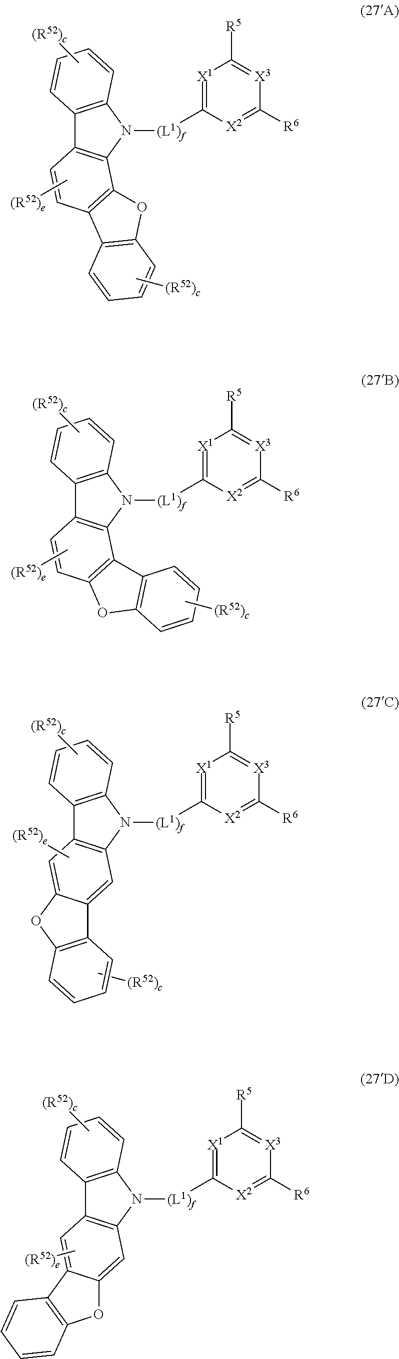

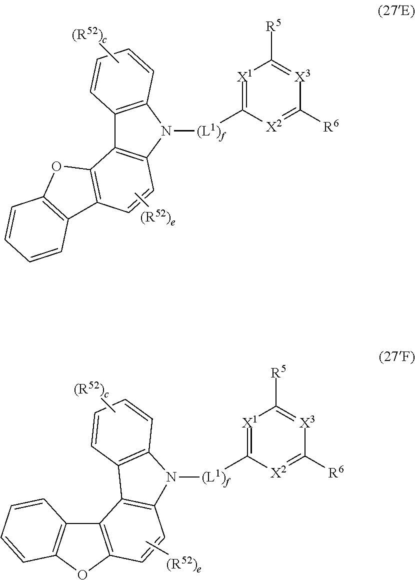

[In the formulae (27′A) to (27′F),

X¹ to X³, R⁵, R⁶, L¹, f, R⁵², and c are as defined in the formula (25).

e is an integer of 0 to 2; when e is 2, a plurality of R⁵² may be identical with or different from each other.]

The compounds represented by the formula (25) are preferably compounds each represented by any one of the formulae (27″A) to (27° F.).

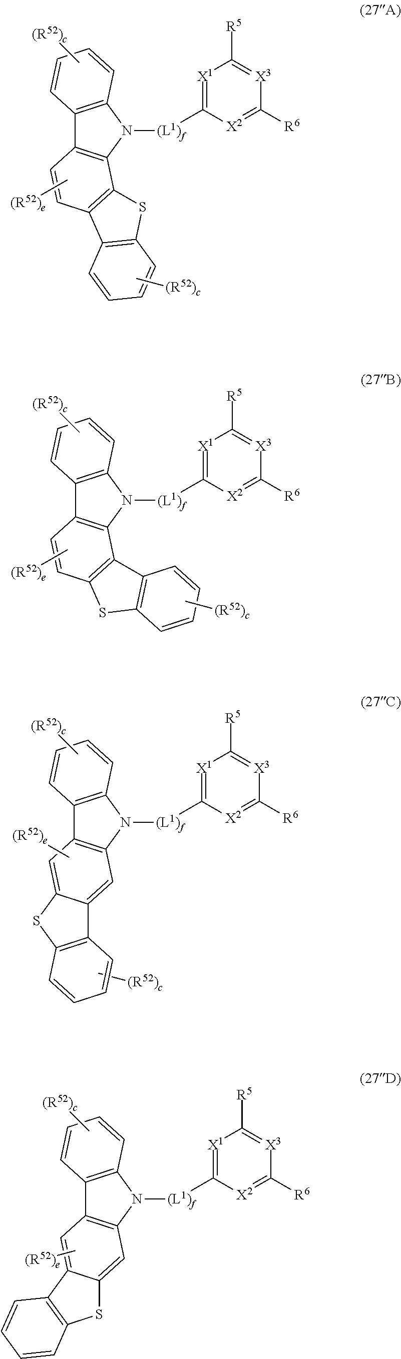

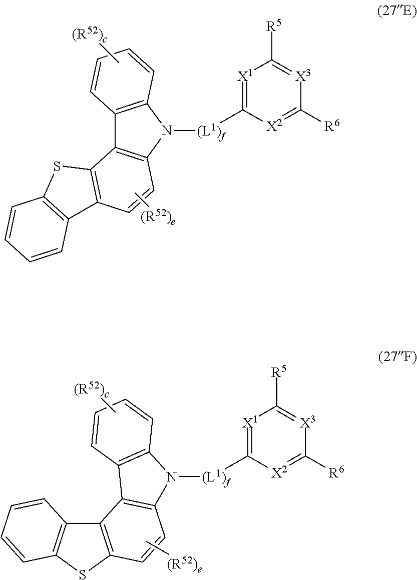

[In the formulae (27″A) to (27° F.),

X¹ to X³, R⁵, R⁶, L¹, f, R⁵², and c are as defined in the formula (25).

e is an integer of 0 to 2; when e is 2, a plurality of R⁵² may be identical or different from each other.]

In another embodiment, the compounds represented by the formula (22) may be compounds represented by the formula (28).

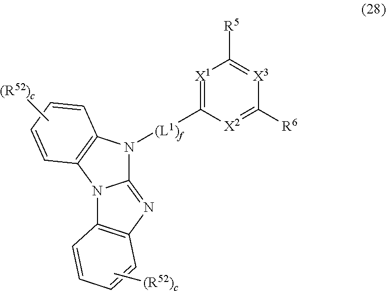

[In the formula (28),

X¹ to X³, R⁵, R⁶, L¹, f, R⁵² and c are as defined in the formula (22).]

In yet another embodiment, the compounds represented by the formula (2) are preferably compounds represented by the formula (30).

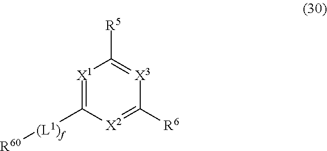

[In the formula (30),

X¹ to X³, R⁵, and R⁶ are as defined in the formula (2).

L¹ is a substituted or unsubstituted arylene group including 6 to 24 ring carbon atoms or a substituted or unsubstituted heteroarylene group including 5 to 30 ring atoms.

f is an integer of 0 to 4; when f is 2 to 4, a plurality of L¹ may be identical with or different from each other, and when f is 0, the group containing X¹ to X³ is bonded with R⁶⁰ via a single bond.

R⁶⁰ is a substituted or unsubstituted condensed aryl group including 10 to 24 ring carbon atoms, or a substituted or unsubstituted condensed heteroaryl group including 9 to 30 ring atoms.]

The substituted or unsubstituted condensed aryl group including 10 to 24 ring carbon atoms as R⁶⁰ is preferably a monovalent residue of a compound represented by the following formula (a1-1) or (a1-2).

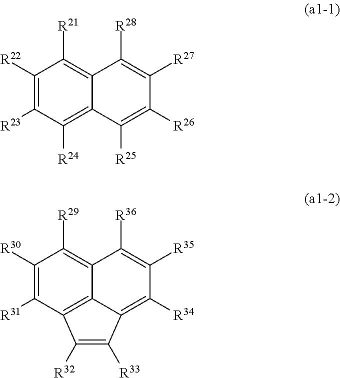

[In the formulae (a1-1) and (a1-2),

R²¹ to R³⁶ are independently a hydrogen atom or a substituent R^{b}; when a plurality of R^{b} are present, they may be identical with or different from each other and two R^{b} selected from the plurality of R^{b} may be bonded with each other to form a ring.]

Examples of the substituent R^{b} are the same as those of the substituent R⁵⁹ in the formula (31) described later.

As examples of the substituted or unsubstituted condensed ring residue including 10 to 24 ring carbon atoms in compounds represented by the formula (a1-1), condensed aromatic rings described below are mentioned. Among them, preference is given to a condensed aromatic ring group in which four or more rings are condensed, and a triphenylenyl group is mentioned as a specific example thereof.

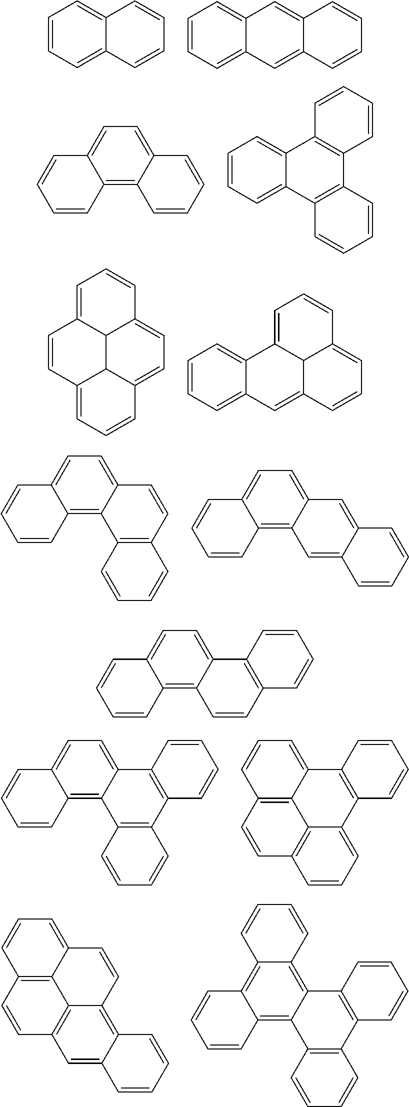

As examples of the substituted or unsubstituted condensed ring residue including 10 to 24 ring carbon atoms in compounds represented by the formula (a1-2), compounds described below are mentioned. Preference is given to a condensed ring group in which four or more rings are condensed, and a fluoranthenyl group is mentioned as a specific example thereof.

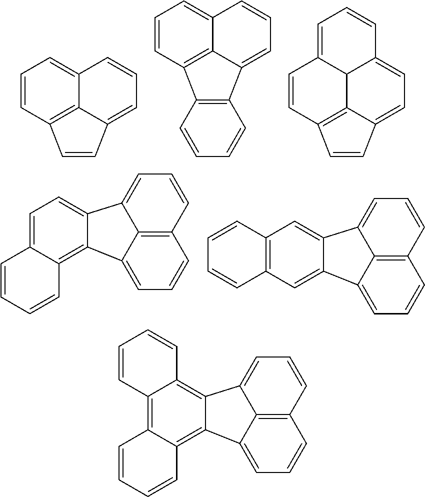

As the substituted or unsubstituted condensed heteroaryl group including 9 to 30 ring atoms as R⁶⁰, heteroaryl groups including 9 to 30 ring atoms are mentioned among the groups described as examples of the heteroaryl above. Particularly, monovalent residues of compounds represented by the following formula (a2) are preferred.

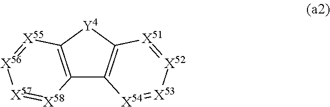

[In the formula (a2),

X⁵¹ to X⁵⁸ are independently CH, C(R^{b}) or N.

R^{b} is a substituent; when a plurality of R^{b} are present, they may be identical with or different from each other and two R^{b} selected from the plurality of R^{b} may be bonded with each other to form a ring.

Y⁴ is an oxygen atom, a sulfur atom, —NR^{d}, or —C(R^{e})(R^{f})—.

R^{d}, R^{e}, and R^{f} are independently a hydrogen atom or a substituent R^{b} and when both R^{e} and R^{f} are R^{b}, they may be bonded with each other to form a ring.]

The substituent R^{b} is as defined above.

The monovalent residues of the compounds represented by the formula (a2) are preferably monovalent residues of compounds represented by the following formula (a2-1).

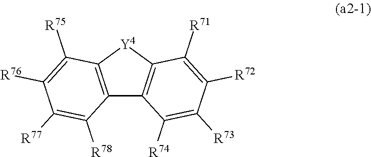

[In the formula (a2-1),

Y⁴ is as defined in the formula (a2).

R⁷¹ to R⁷⁸ are independently a hydrogen atom or a substituent R^{b}; when a plurality of R^{b} are present, they may be identical with or different from each other and two R^{b} selected from the plurality of R^{b} may be bonded with each other to form a ring.]

In the compounds represented by the formula (a2-1), Y⁴ is preferably an oxygen atom, a sulfur atom, NH, C(CH₃)₂, for example.

It is preferred that any one of R⁷¹ to R⁷⁸ form a single bond with L¹.

The compounds represented by the formula (30) are preferably compounds represented by the formula (31).

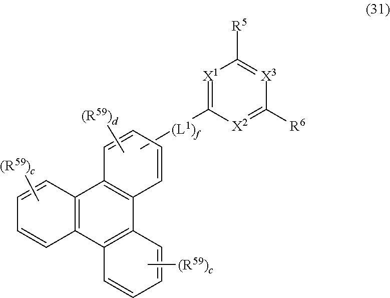

[In the formula (31),

X¹ to X³, R⁵, R⁶, L¹, and f are as defined in the formula (30).

R⁵⁹ is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, or —P(═O)R¹⁴R¹⁵.

R¹⁴ and R¹⁵ are as defined in the formula (2).

c is an integer of 0 to 4 and d is an integer of 0 to 3; when c is 2 to 4 or d is 2 or 3, a plurality of R⁵⁹ may be identical with or different from each other and adjacent R⁵⁹ may be bonded with each other to form a ring.]

In yet still another embodiment, the compounds represented by the formula (30) are preferably compounds represented by the formula (32).

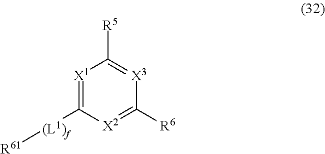

[In the formula (32),

X¹ to X³, R⁵, R⁶, L¹ and f are as defined in the formula (30).

R⁶¹ is a substituted or unsubstituted condensed heteroaryl group including 9 to 30 ring atoms and containing no nitrogen atom.]

Among the groups that are described as the examples of the heteroaryl group above, condensed heteroaryl groups including 9 to 30 ring atoms and containing no nitrogen atom are mentioned as examples of the condensed heteroaryl group including 9 to 30 ring atoms and containing no nitrogen atom in R⁶¹.

The compounds represented by the formula (32) are preferably compounds represented by the formula (33).

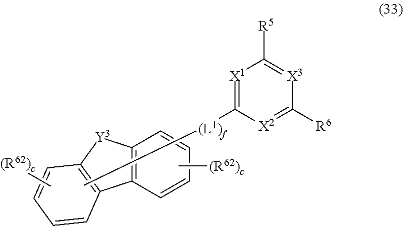

[In the formula (33),

X¹ to X³, R⁵, R⁶, L¹, and f are as defined in the formula (32).

R⁶² is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, or —P(═O)R¹⁴R¹⁵.

R¹⁴ and R¹⁵ are as defined in the formula (2).

c is an integer of 0 to 4; when c is 2 to 4, a plurality of R⁵² may be identical with or different from each other and adjacent R⁵² may be bonded with each other to form a ring.

Y³ is an oxygen atom or a sulfur atom.]

It is preferred that f in the formulae (21) to (33) be 0 or 1.

R⁵ and R⁶ in the formulae (21) to (33) are independently a substituted or unsubstituted aryl group including 6 to 24 ring carbon atoms, or a substituted or unsubstituted heterocyclic group, more preferably a substituted or unsubstituted phenyl group, a substituted or unsubstituted biphenyl group, a substituted or unsubstituted terphenyl group, a substituted or unsubstituted dibenzofuranyl group, a substituted or unsubstituted dibenzothiophenyl group, or a substituted or unsubstituted carbazolyl group.

The following are specific examples of the compounds represented by the formula (2), but the compounds are not limited to the examples.

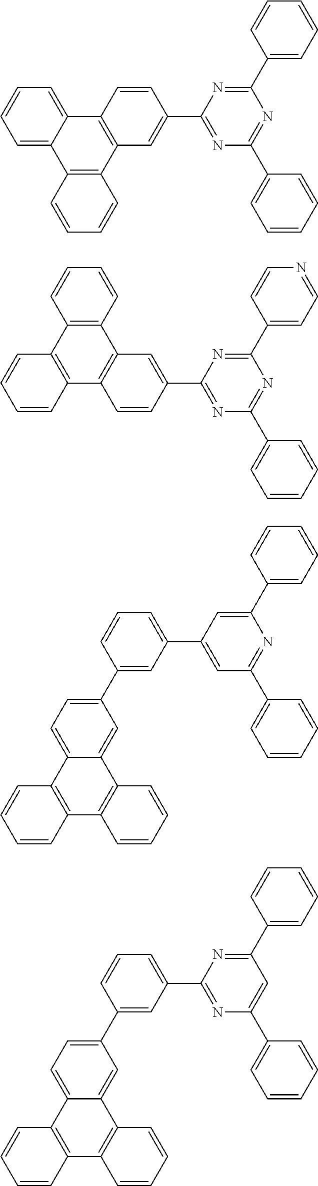

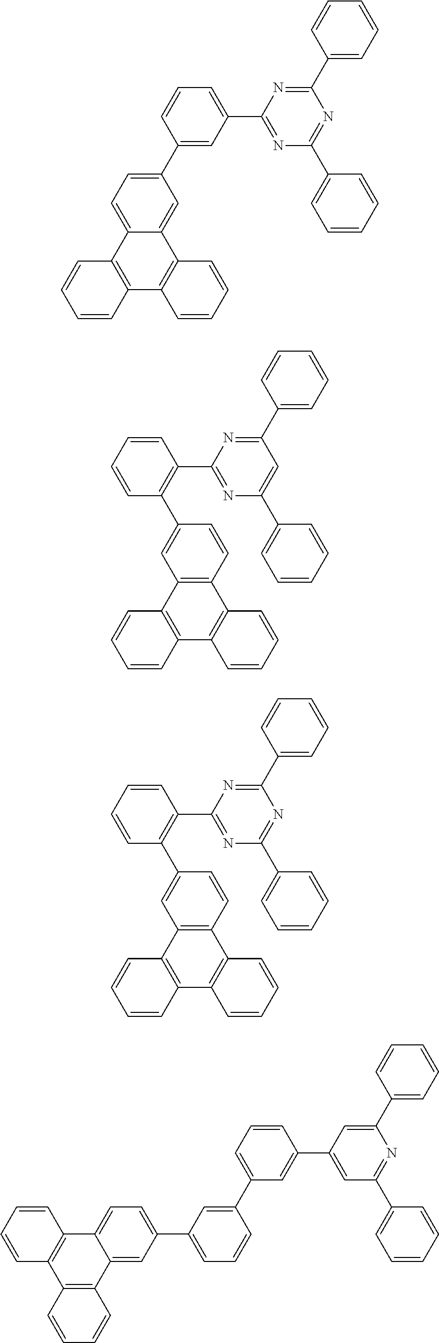

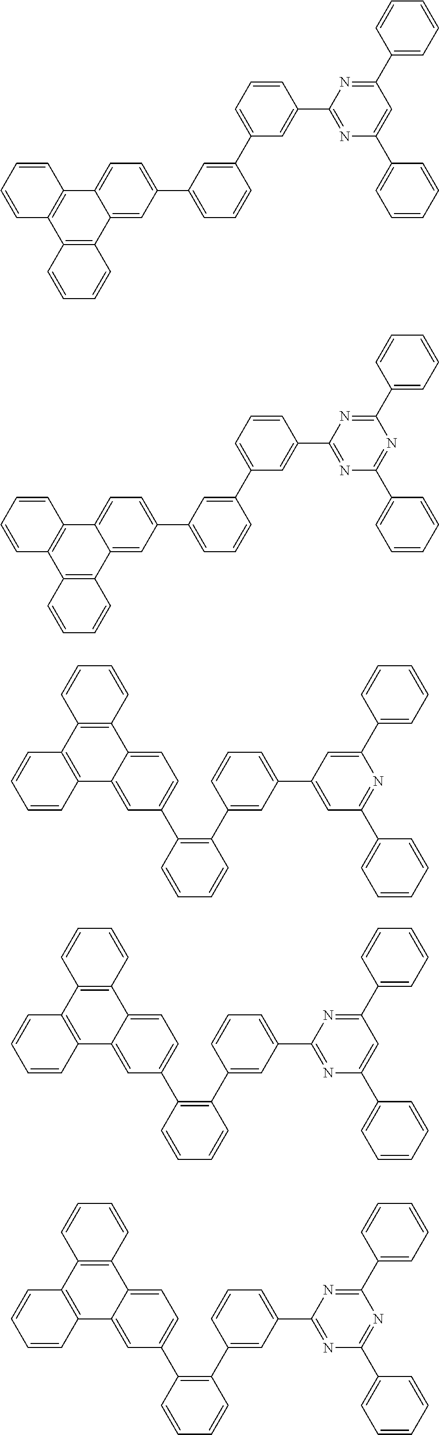

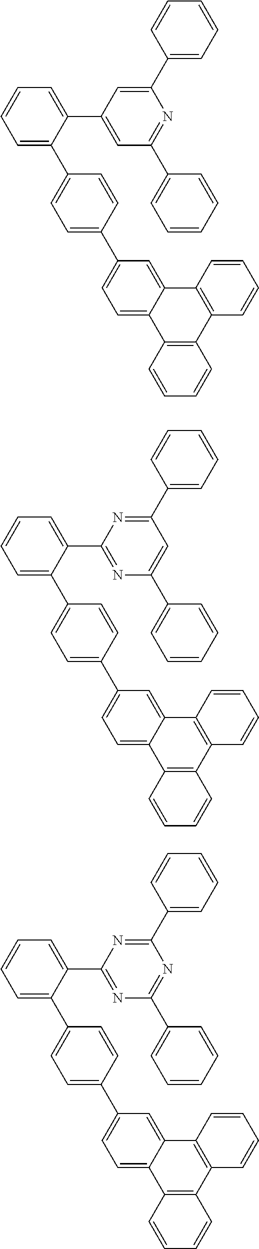

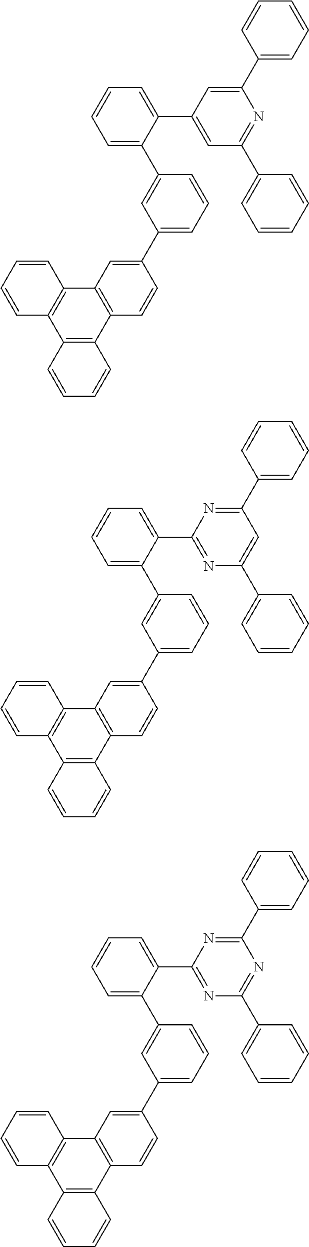 [SMILES: c1ccc(-c2cc(-c3ccccc3-c3cccc(-c4ccc5c6ccccc6c6ccccc6c5c4)c3)cc(-c3ccccc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1]

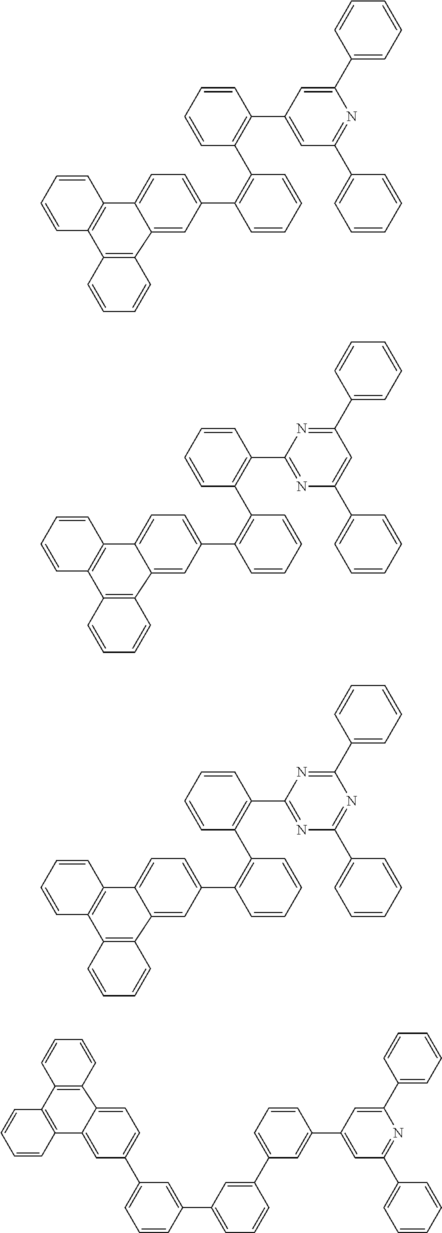 [SMILES: c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1 ; c1ccc(-c2cc(-c3cccc(-c4cccc(-c5cccc(-c6ccc7c8ccccc8c8ccccc8c7c6)c5)c4)c3)cc(-c3ccccc3)n2)cc1]

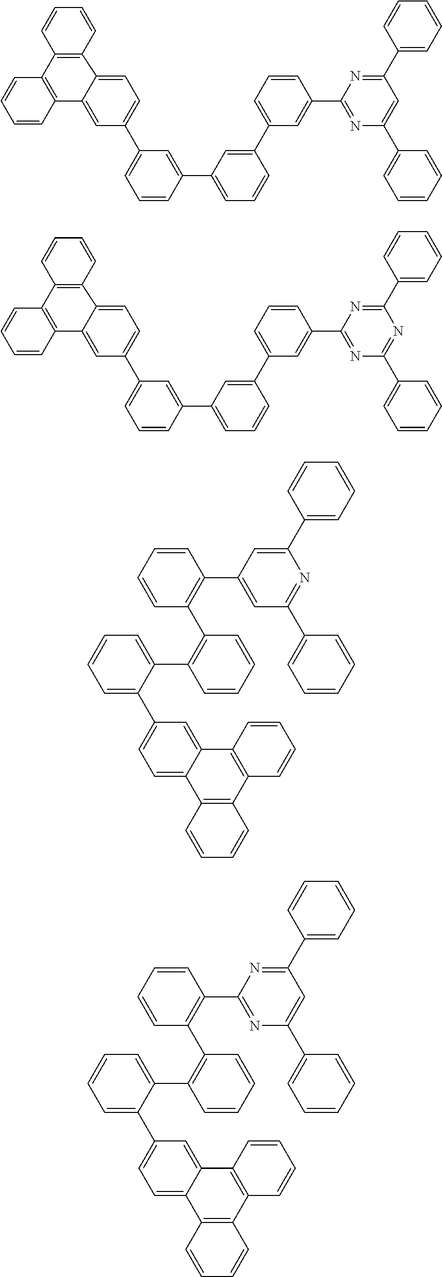

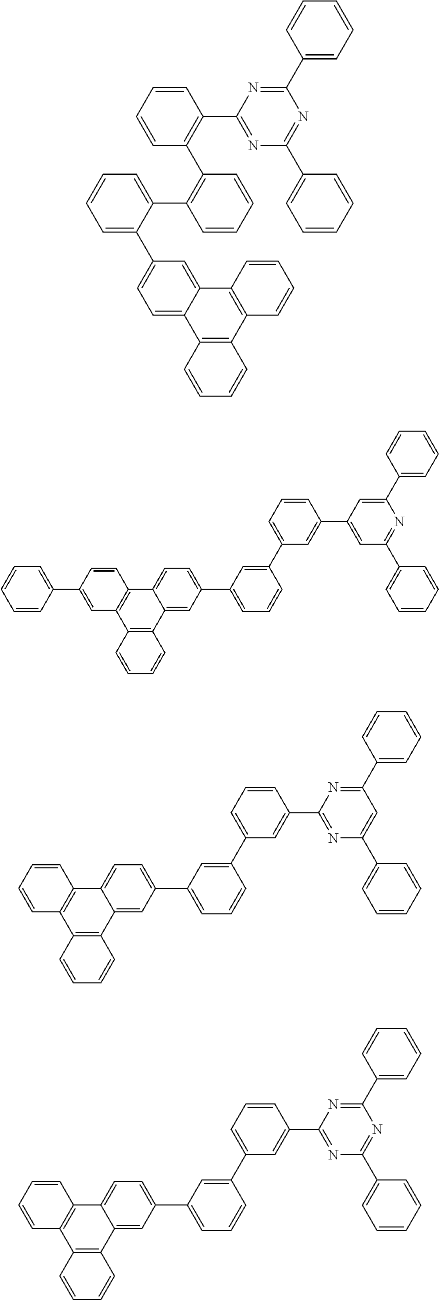 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1]

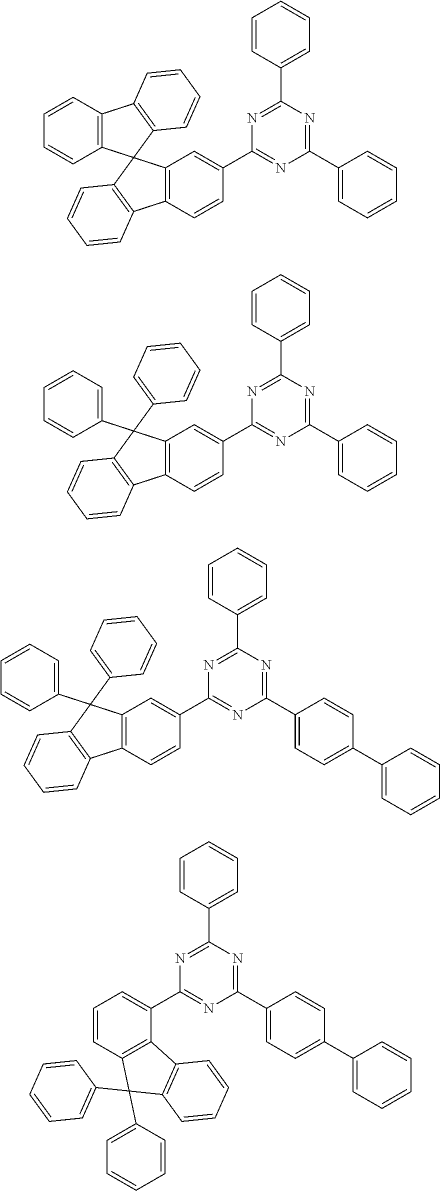 [SMILES: c1ccc(-c2nc(-c3ccccc3)nc(-c3ccc4c(c3)C3(c5ccccc5-c5ccccc53)c3ccccc3-4)n2)cc1 ; c1ccc(-c2nc(-c3ccccc3)nc(-c3ccc4c(c3)C(c3ccccc3)(c3ccccc3)c3ccccc3-4)n2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1 ; c1ccc(-c2ccc(-c3nc(-c4ccccc4)nc(-c4cccc5c4-c4ccccc4C5(c4ccccc4)c4ccccc4)n3)cc2)cc1]

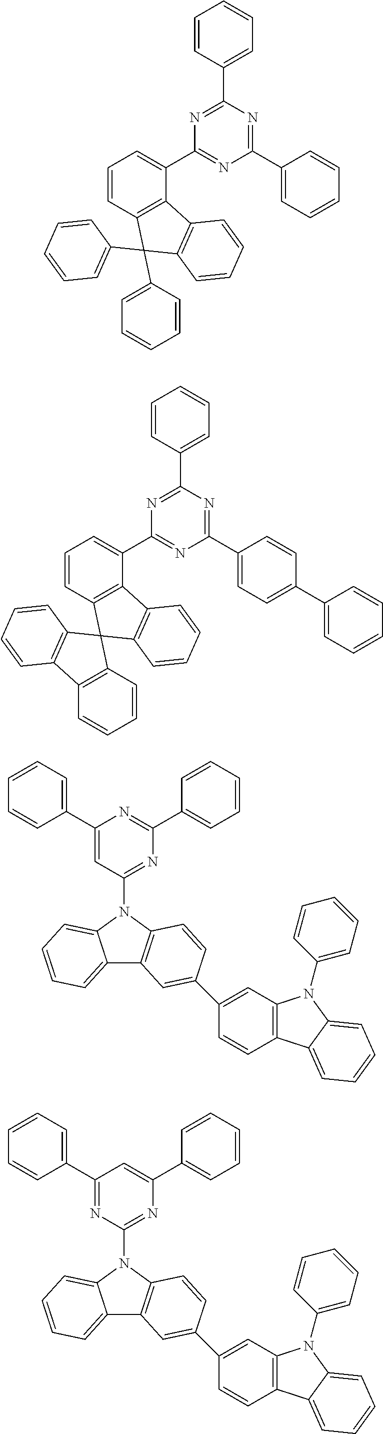 [SMILES: c1ccc(-c2nc(-c3ccccc3)nc(-c3cccc4c3-c3ccccc3C4(c3ccccc3)c3ccccc3)n2)cc1 ; c1ccc(-c2ccc(-c3nc(-c4ccccc4)nc(-c4cccc5c4-c4ccccc4C54c5ccccc5-c5ccccc54)n3)cc2)cc1 ; c1ccc(-c2cc(-n3c4ccccc4c4cc(-c5ccc6c7ccccc7n(-c7ccccc7)c6c5)ccc43)nc(-c3ccccc3)n2)cc1 ; c1ccc(-c2cc(-c3ccccc3)nc(-n3c4ccccc4c4cc(-c5ccc6c7ccccc7n(-c7ccccc7)c6c5)ccc43)n2)cc1]

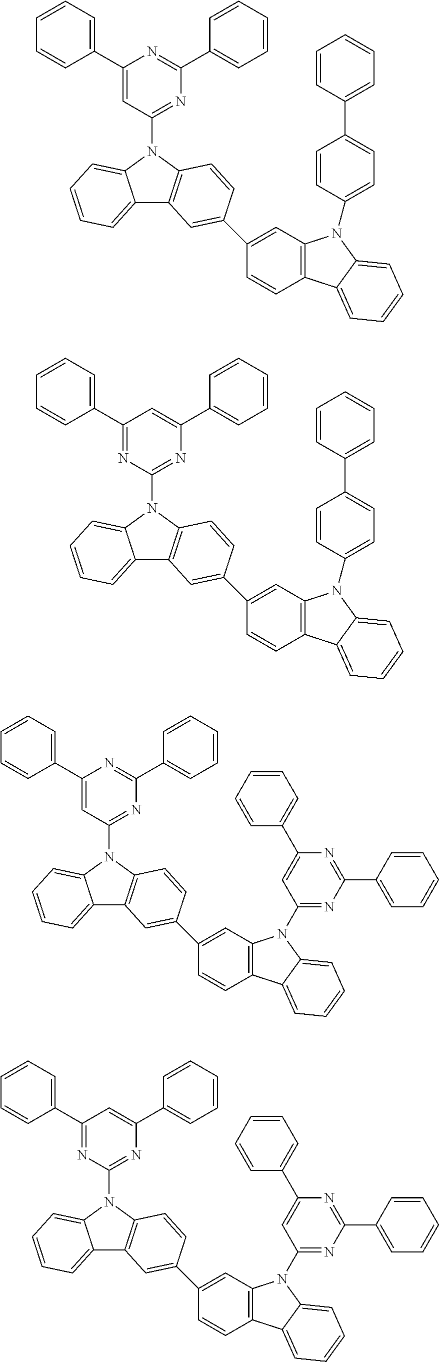 [SMILES: c1ccc(-c2ccc(-n3c4ccccc4c4ccc(-c5ccc6c(c5)c5ccccc5n6-c5cc(-c6ccccc6)nc(-c6ccccc6)n5)cc43)cc2)cc1 ; c1ccc(-c2ccc(-n3c4ccccc4c4ccc(-c5ccc6c(c5)c5ccccc5n6-c5nc(-c6ccccc6)cc(-c6ccccc6)n5)cc43)cc2)cc1 ; c1ccc(-c2cc(-n3c4ccccc4c4cc(-c5ccc6c7ccccc7n(-c7cc(-c8ccccc8)nc(-c8ccccc8)n7)c6c5)ccc43)nc(-c3ccccc3)n2)cc1 ; c1ccc(-c2cc(-n3c4ccccc4c4ccc(-c5ccc6c(c5)c5ccccc5n6-c5nc(-c6ccccc6)cc(-c6ccccc6)n5)cc43)nc(-c3ccccc3)n2)cc1]

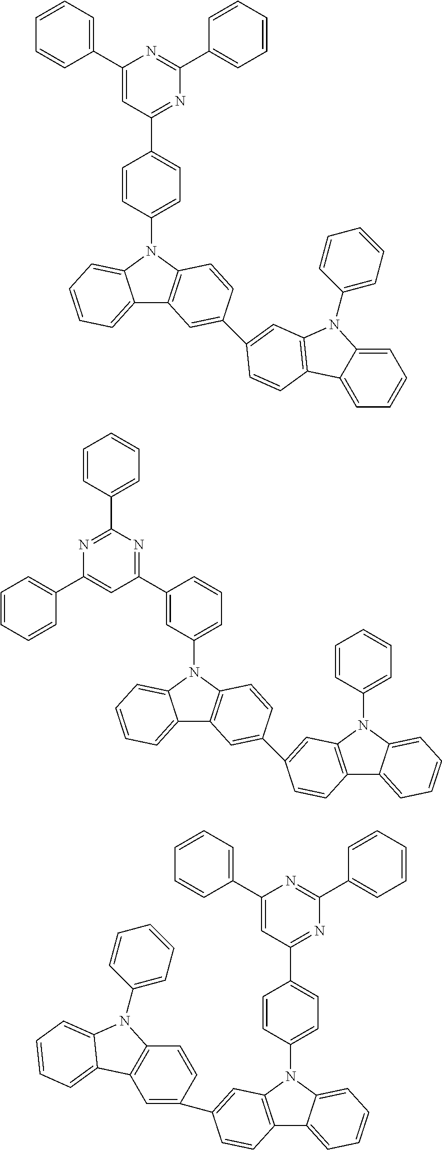

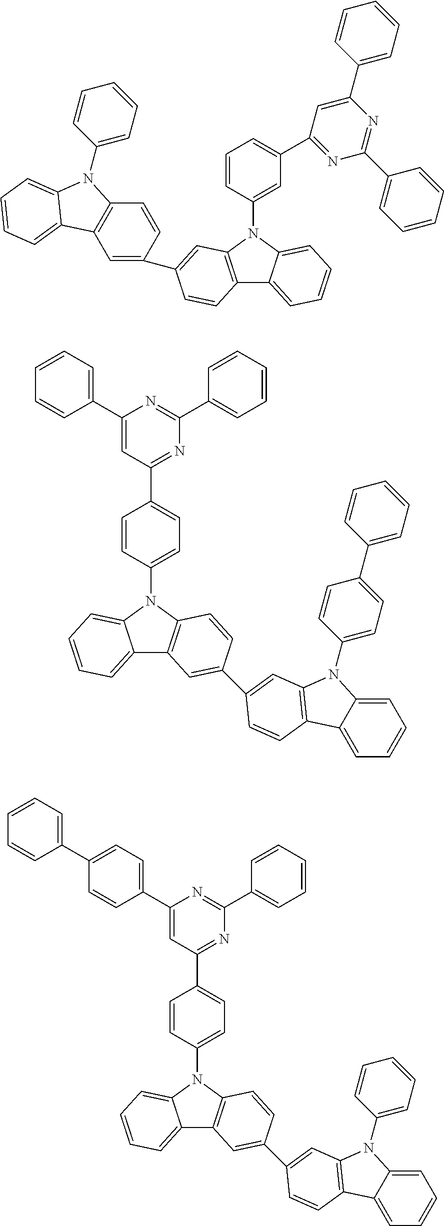 [SMILES: c1ccc(-c2cc(-c3cccc(-n4c5ccccc5c5ccc(-c6ccc7c(c6)c6ccccc6n7-c6ccccc6)cc54)c3)nc(-c3ccccc3)n2)cc1 ; c1ccc(-c2ccc(-n3c4ccccc4c4ccc(-c5ccc6c(c5)c5ccccc5n6-c5ccc(-c6cc(-c7ccccc7)nc(-c7ccccc7)n6)cc5)cc43)cc2)cc1 ; c1ccc(-c2ccc(-c3cc(-c4ccc(-n5c6ccccc6c6cc(-c7ccc8c9ccccc9n(-c9ccccc9)c8c7)ccc65)cc4)nc(-c4ccccc4)n3)cc2)cc1]

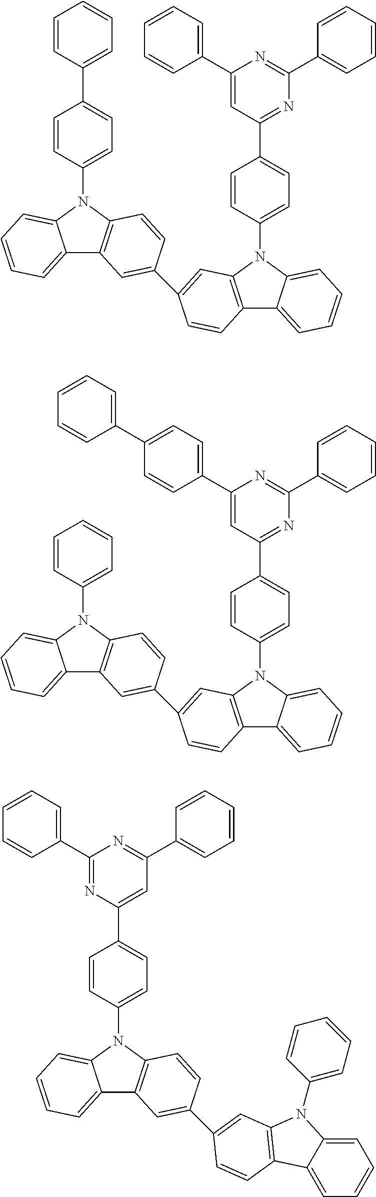

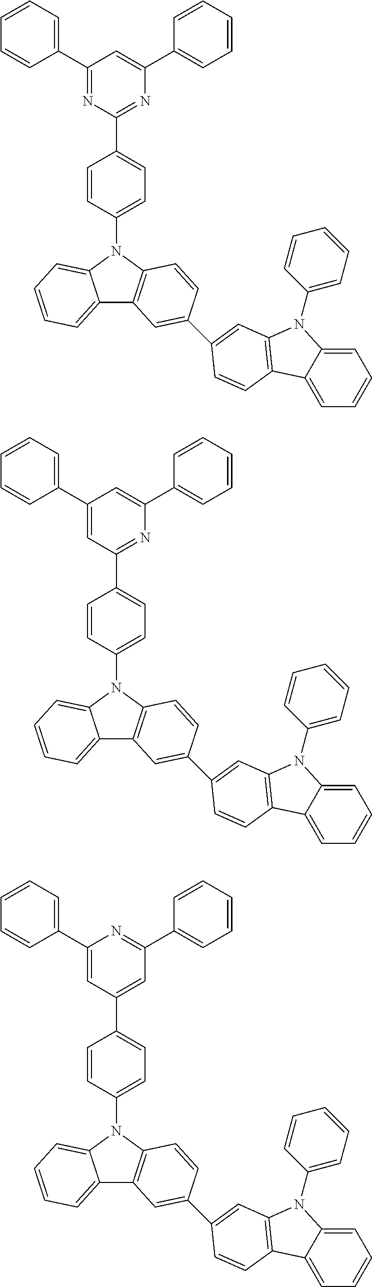 [SMILES: c1ccc(-c2cc(-c3ccccc3)nc(-c3ccc(-n4c5ccccc5c5cc(-c6ccc7c8ccccc8n(-c8ccccc8)c7c6)ccc54)cc3)n2)cc1 ; c1ccc(-c2cc(-c3ccccc3)nc(-c3ccc(-n4c5ccccc5c5cc(-c6ccc7c8ccccc8n(-c8ccccc8)c7c6)ccc54)cc3)c2)cc1 ; c1ccc(-c2cc(-c3ccc(-n4c5ccccc5c5cc(-c6ccc7c8ccccc8n(-c8ccccc8)c7c6)ccc54)cc3)cc(-c3ccccc3)n2)cc1]

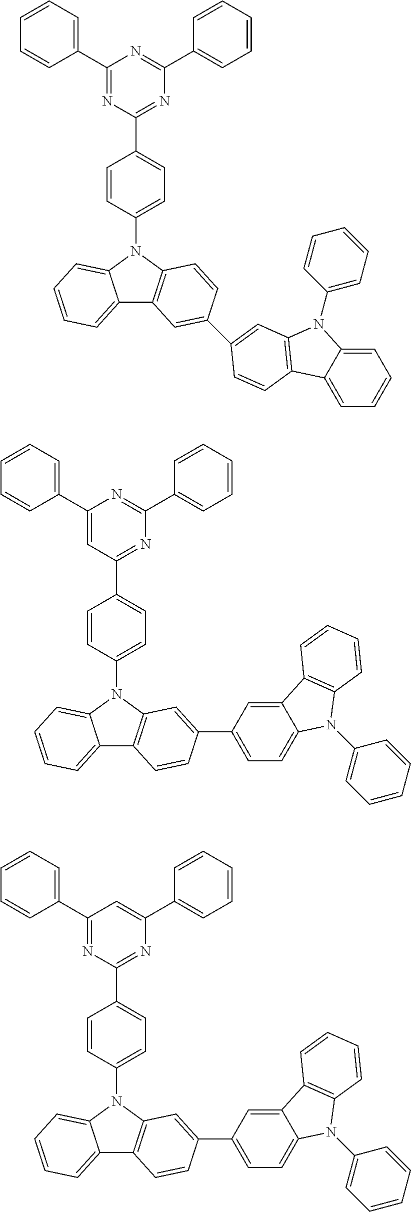 [SMILES: c1ccc(-c2nc(-c3ccccc3)nc(-c3ccc(-n4c5ccccc5c5cc(-c6ccc7c8ccccc8n(-c8ccccc8)c7c6)ccc54)cc3)n2)cc1 ; c1ccc(-c2cc(-c3ccc(-n4c5ccccc5c5ccc(-c6ccc7c(c6)c6ccccc6n7-c6ccccc6)cc54)cc3)nc(-c3ccccc3)n2)cc1 ; c1ccc(-c2cc(-c3ccccc3)nc(-c3ccc(-n4c5ccccc5c5ccc(-c6ccc7c(c6)c6ccccc6n7-c6ccccc6)cc54)cc3)n2)cc1]

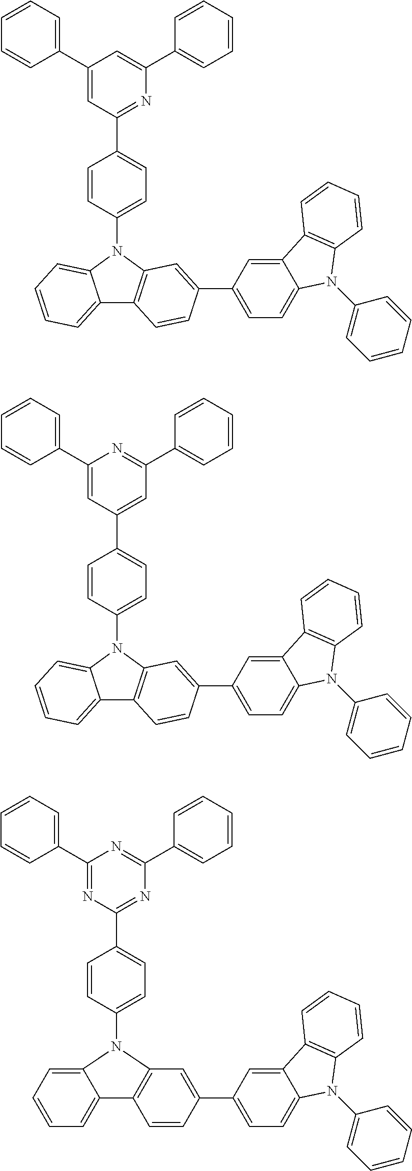 [SMILES: c1ccc(-c2cc(-c3ccccc3)nc(-c3ccc(-n4c5ccccc5c5ccc(-c6ccc7c(c6)c6ccccc6n7-c6ccccc6)cc54)cc3)c2)cc1 ; c1ccc(-c2cc(-c3ccc(-n4c5ccccc5c5ccc(-c6ccc7c(c6)c6ccccc6n7-c6ccccc6)cc54)cc3)cc(-c3ccccc3)n2)cc1 ; c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1]

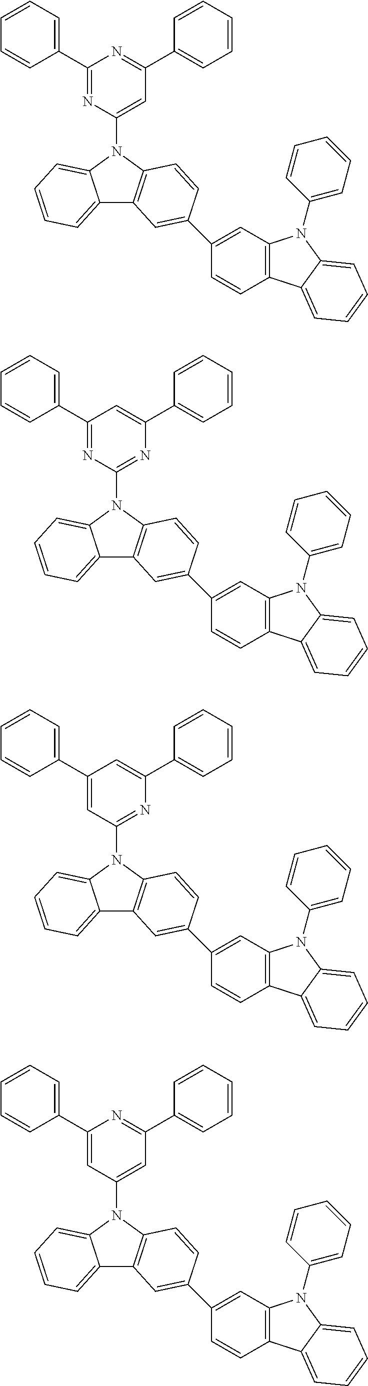 [SMILES: c1ccc(-c2cc(-n3c4ccccc4c4cc(-c5ccc6c7ccccc7n(-c7ccccc7)c6c5)ccc43)nc(-c3ccccc3)n2)cc1 ; c1ccc(-c2cc(-c3ccccc3)nc(-n3c4ccccc4c4cc(-c5ccc6c7ccccc7n(-c7ccccc7)c6c5)ccc43)n2)cc1 ; c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1]

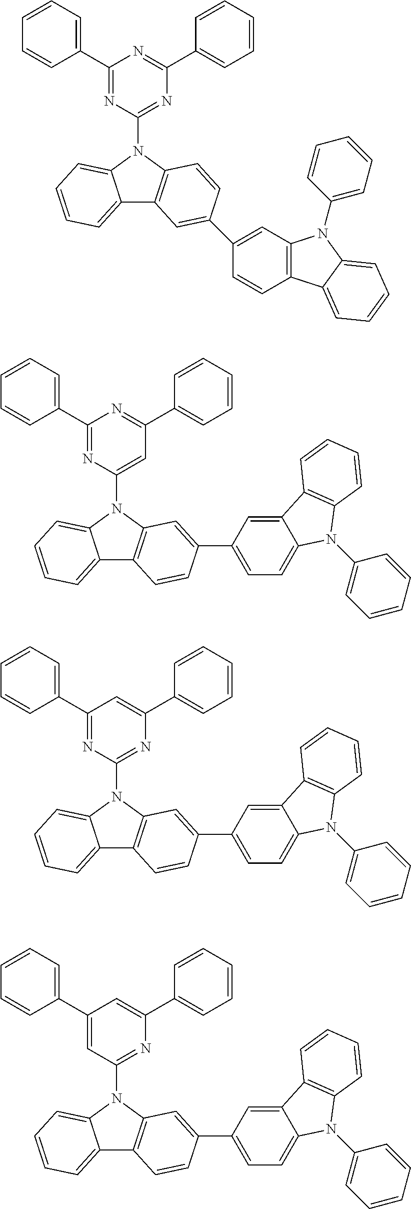 [SMILES: c1ccc(-c2nc(-c3ccccc3)nc(-n3c4ccccc4c4cc(-c5ccc6c7ccccc7n(-c7ccccc7)c6c5)ccc43)n2)cc1 ; c1ccc(-c2cc(-n3c4ccccc4c4ccc(-c5ccc6c(c5)c5ccccc5n6-c5ccccc5)cc43)nc(-c3ccccc3)n2)cc1 ; c1ccc(-c2cc(-c3ccccc3)nc(-n3c4ccccc4c4ccc(-c5ccc6c(c5)c5ccccc5n6-c5ccccc5)cc43)n2)cc1 ; c1ccc(-c2cc(-c3ccccc3)nc(-n3c4ccccc4c4ccc(-c5ccc6c(c5)c5ccccc5n6-c5ccccc5)cc43)c2)cc1]

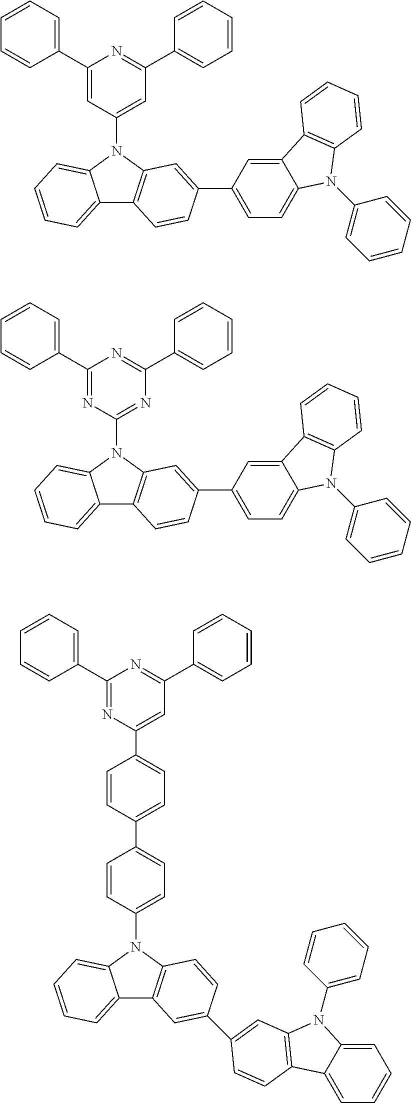 [SMILES: c1ccc(-c2cc(-n3c4ccccc4c4ccc(-c5ccc6c(c5)c5ccccc5n6-c5ccccc5)cc43)cc(-c3ccccc3)n2)cc1 ; c1ccc(-c2nc(-c3ccccc3)nc(-n3c4ccccc4c4ccc(-c5ccc6c(c5)c5ccccc5n6-c5ccccc5)cc43)n2)cc1 ; c1ccc(-c2cc(-c3ccc(-c4ccc(-n5c6ccccc6c6cc(-c7ccc8c9ccccc9n(-c9ccccc9)c8c7)ccc65)cc4)cc3)nc(-c3ccccc3)n2)cc1]

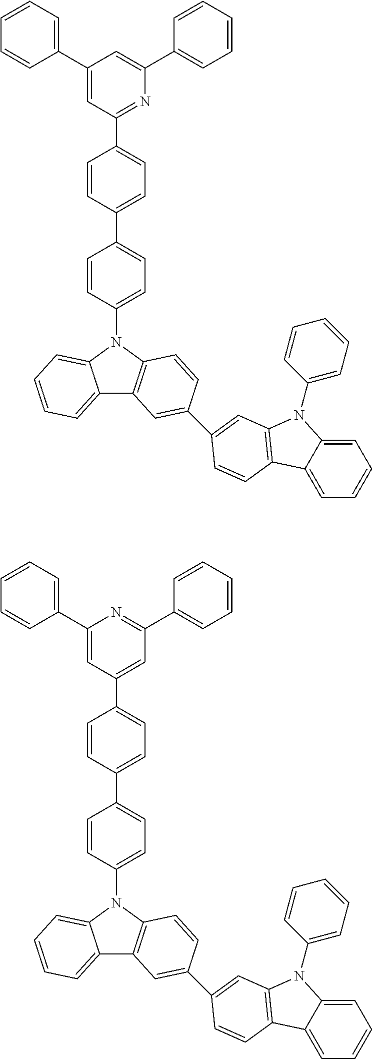 [SMILES: c1ccc(-c2cc(-c3ccccc3)nc(-c3ccc(-c4ccc(-n5c6ccccc6c6cc(-c7ccc8c9ccccc9n(-c9ccccc9)c8c7)ccc65)cc4)cc3)c2)cc1 ; c1ccc(-c2cc(-c3ccc(-c4ccc(-n5c6ccccc6c6cc(-c7ccc8c9ccccc9n(-c9ccccc9)c8c7)ccc65)cc4)cc3)cc(-c3ccccc3)n2)cc1]

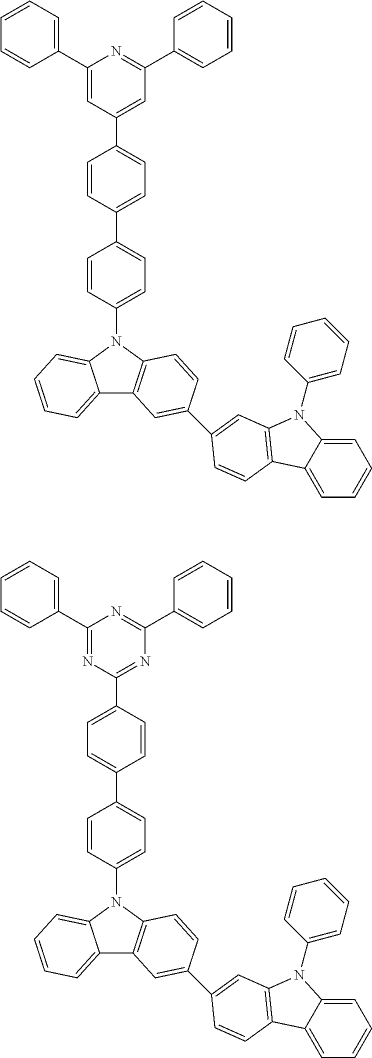 [SMILES: c1ccc(-c2cc(-c3ccc(-c4ccc(-n5c6ccccc6c6cc(-c7ccc8c9ccccc9n(-c9ccccc9)c8c7)ccc65)cc4)cc3)cc(-c3ccccc3)n2)cc1 ; c1ccc(-c2nc(-c3ccccc3)nc(-c3ccc(-c4ccc(-n5c6ccccc6c6cc(-c7ccc8c9ccccc9n(-c9ccccc9)c8c7)ccc65)cc4)cc3)n2)cc1]

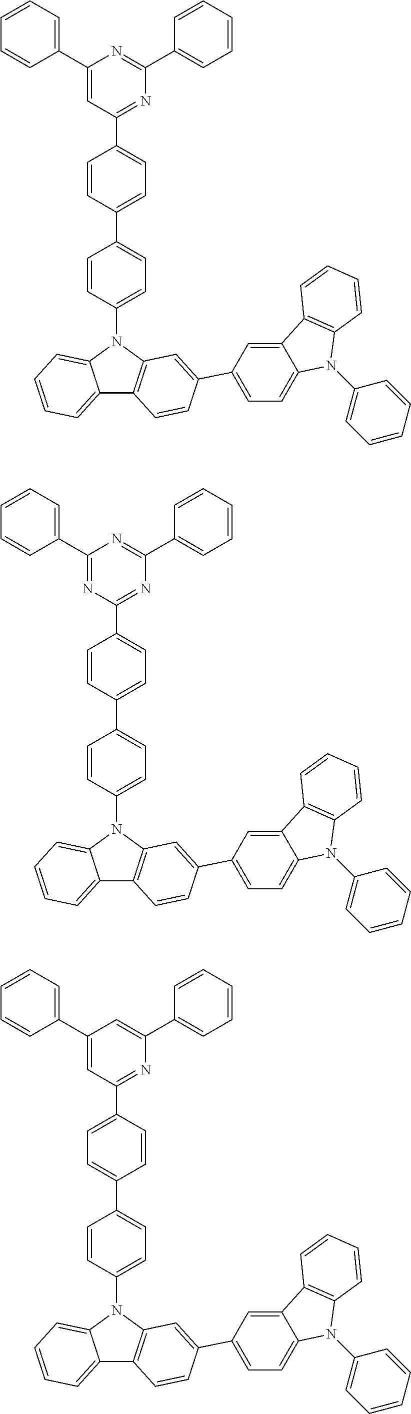

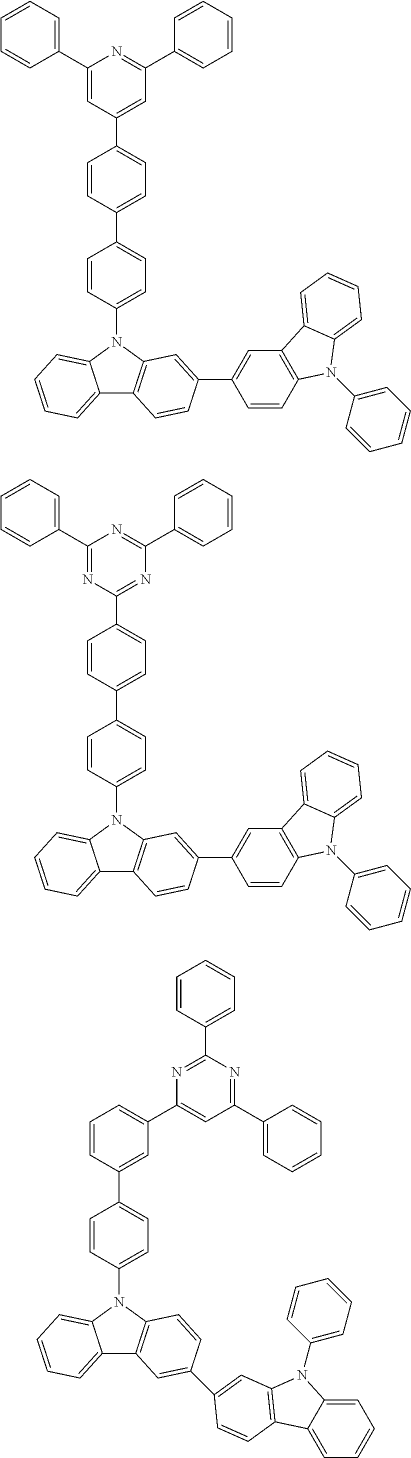

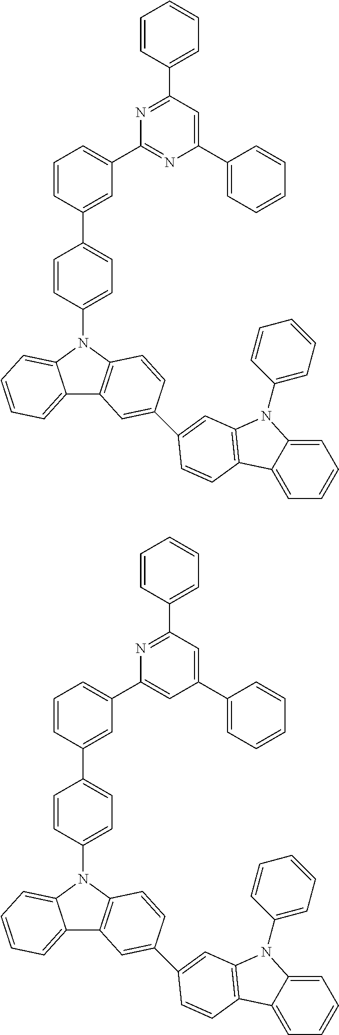 [SMILES: c1ccc(-c2cc(-c3ccccc3)nc(-c3cccc(-c4ccc(-n5c6ccccc6c6cc(-c7ccc8c9ccccc9n(-c9ccccc9)c8c7)ccc65)cc4)c3)n2)cc1 ; c1ccc(-c2cc(-c3ccccc3)nc(-c3cccc(-c4ccc(-n5c6ccccc6c6cc(-c7ccc8c9ccccc9n(-c9ccccc9)c8c7)ccc65)cc4)c3)c2)cc1]

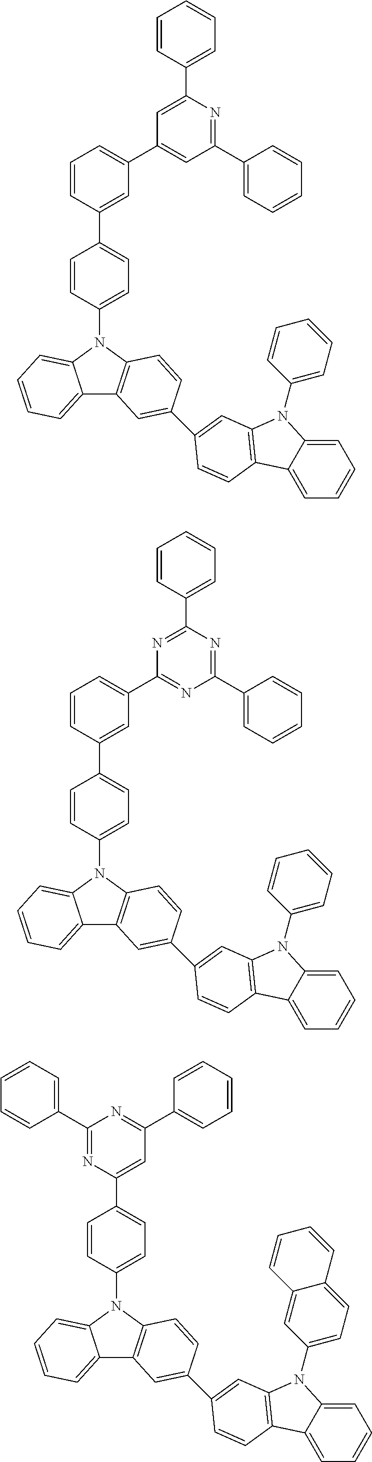 [SMILES: c1ccc(-c2cc(-c3cccc(-c4ccc(-n5c6ccccc6c6cc(-c7ccc8c9ccccc9n(-c9ccccc9)c8c7)ccc65)cc4)c3)cc(-c3ccccc3)n2)cc1 ; c1ccc(-c2nc(-c3ccccc3)nc(-c3cccc(-c4ccc(-n5c6ccccc6c6cc(-c7ccc8c9ccccc9n(-c9ccccc9)c8c7)ccc65)cc4)c3)n2)cc1 ; c1ccc(-c2cc(-c3ccc(-n4c5ccccc5c5cc(-c6ccc7c8ccccc8n(-c8ccc9ccccc9c8)c7c6)ccc54)cc3)nc(-c3ccccc3)n2)cc1]

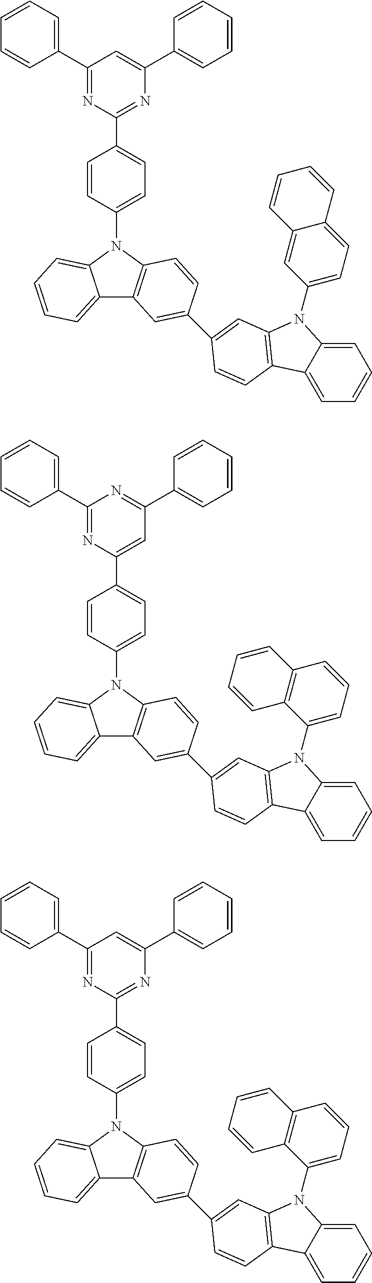 [SMILES: c1ccc(-c2cc(-c3ccccc3)nc(-c3ccc(-n4c5ccccc5c5cc(-c6ccc7c8ccccc8n(-c8ccc9ccccc9c8)c7c6)ccc54)cc3)n2)cc1 ; c1ccc(-c2cc(-c3ccc(-n4c5ccccc5c5cc(-c6ccc7c8ccccc8n(-c8cccc9ccccc89)c7c6)ccc54)cc3)nc(-c3ccccc3)n2)cc1 ; c1ccc(-c2cc(-c3ccccc3)nc(-c3ccc(-n4c5ccccc5c5cc(-c6ccc7c8ccccc8n(-c8cccc9ccccc89)c7c6)ccc54)cc3)n2)cc1]

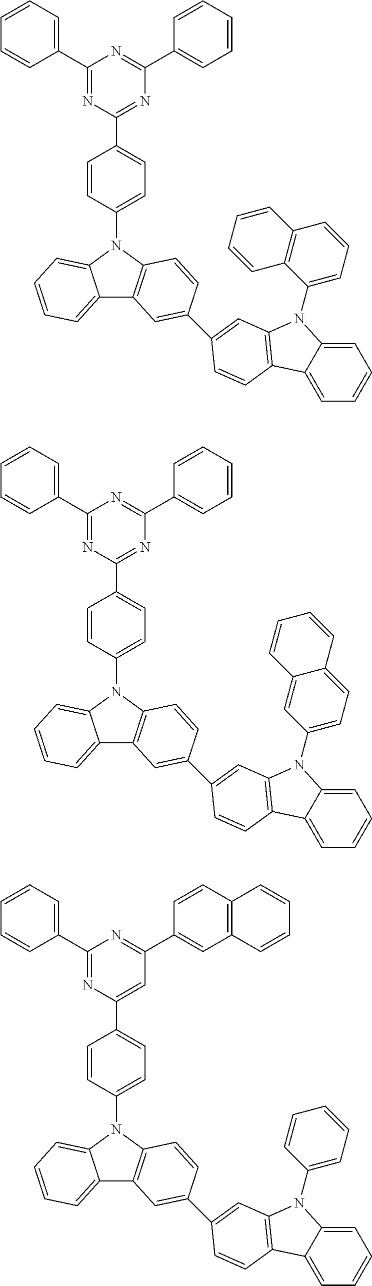 [SMILES: c1ccc(-c2nc(-c3ccccc3)nc(-c3ccc(-n4c5ccccc5c5cc(-c6ccc7c8ccccc8n(-c8cccc9ccccc89)c7c6)ccc54)cc3)n2)cc1 ; c1ccc(-c2nc(-c3ccccc3)nc(-c3ccc(-n4c5ccccc5c5cc(-c6ccc7c8ccccc8n(-c8ccc9ccccc9c8)c7c6)ccc54)cc3)n2)cc1 ; c1ccc(-c2nc(-c3ccc(-n4c5ccccc5c5cc(-c6ccc7c8ccccc8n(-c8ccccc8)c7c6)ccc54)cc3)cc(-c3ccc4ccccc4c3)n2)cc1]

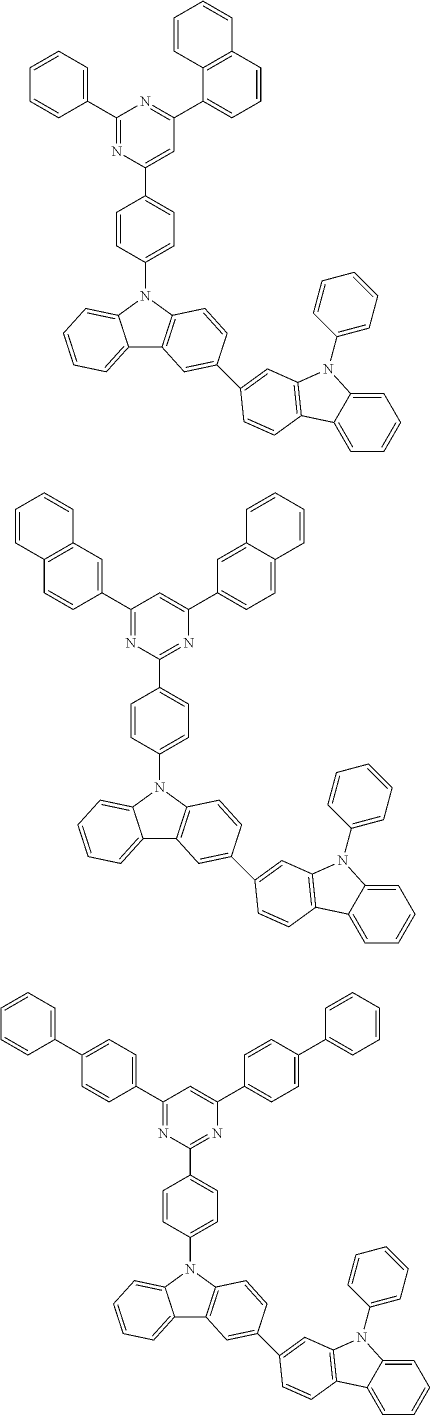 [SMILES: c1ccc(-c2nc(-c3ccc(-n4c5ccccc5c5cc(-c6ccc7c8ccccc8n(-c8ccccc8)c7c6)ccc54)cc3)cc(-c3cccc4ccccc34)n2)cc1 ; c1ccc(-n2c3ccccc3c3ccc(-c4ccc5c(c4)c4ccccc4n5-c4ccc(-c5nc(-c6ccc7ccccc7c6)cc(-c6ccc7ccccc7c6)n5)cc4)cc32)cc1 ; c1ccc(-c2ccc(-c3cc(-c4ccc(-c5ccccc5)cc4)nc(-c4ccc(-n5c6ccccc6c6cc(-c7ccc8c9ccccc9n(-c9ccccc9)c8c7)ccc65)cc4)n3)cc2)cc1]

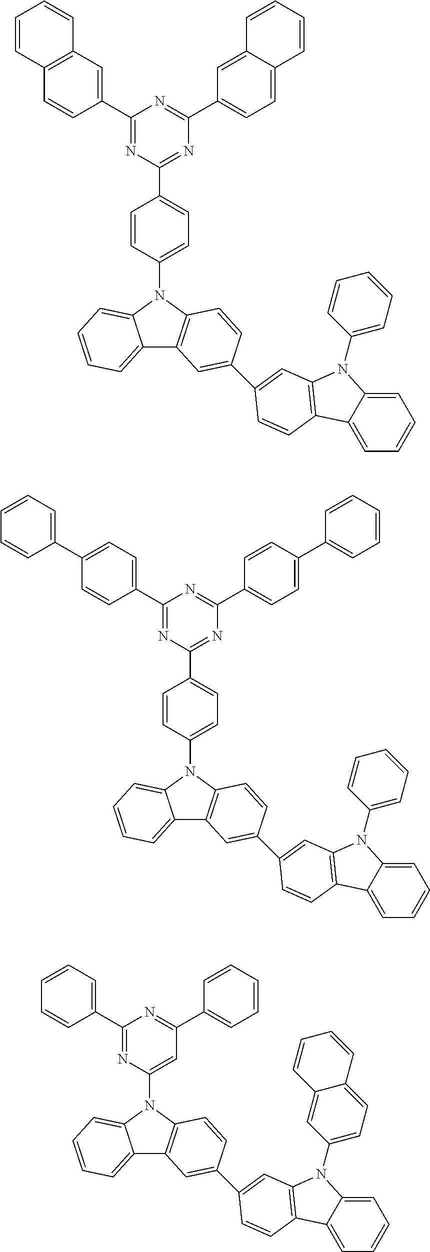 [SMILES: c1ccc(-n2c3ccccc3c3ccc(-c4ccc5c(c4)c4ccccc4n5-c4ccc(-c5nc(-c6ccc7ccccc7c6)nc(-c6ccc7ccccc7c6)n5)cc4)cc32)cc1 ; c1ccc(-c2ccc(-c3nc(-c4ccc(-c5ccccc5)cc4)nc(-c4ccc(-n5c6ccccc6c6cc(-c7ccc8c9ccccc9n(-c9ccccc9)c8c7)ccc65)cc4)n3)cc2)cc1 ; c1ccc(-c2cc(-n3c4ccccc4c4cc(-c5ccc6c7ccccc7n(-c7ccc8ccccc8c7)c6c5)ccc43)nc(-c3ccccc3)n2)cc1]

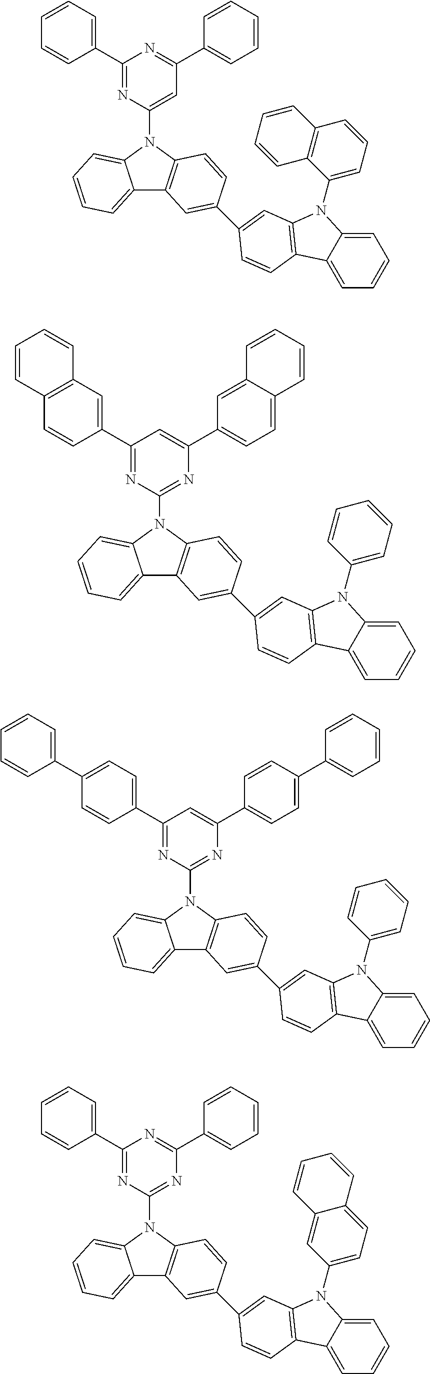

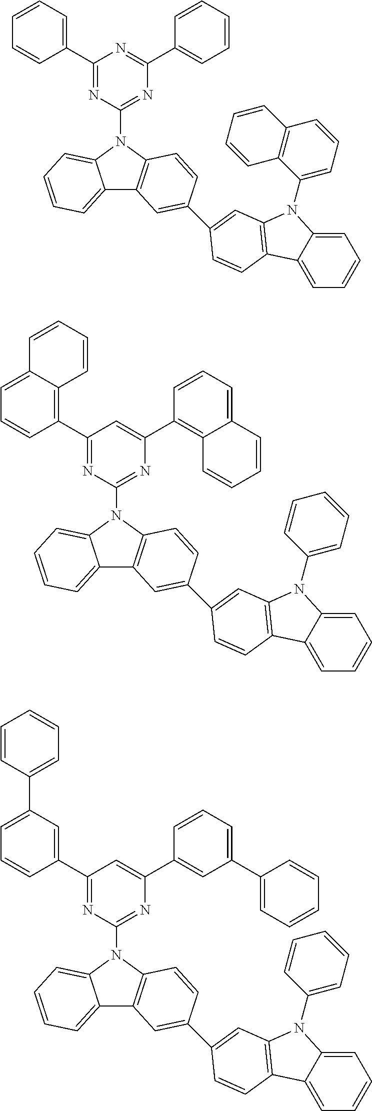 [SMILES: c1ccc(-c2nc(-c3ccccc3)nc(-n3c4ccccc4c4cc(-c5ccc6c7ccccc7n(-c7cccc8ccccc78)c6c5)ccc43)n2)cc1 ; c1ccc(-n2c3ccccc3c3ccc(-c4ccc5c(c4)c4ccccc4n5-c4nc(-c5cccc6ccccc56)cc(-c5cccc6ccccc56)n4)cc32)cc1 ; c1ccc(-c2cccc(-c3cc(-c4cccc(-c5ccccc5)c4)nc(-n4c5ccccc5c5cc(-c6ccc7c8ccccc8n(-c8ccccc8)c7c6)ccc54)n3)c2)cc1]

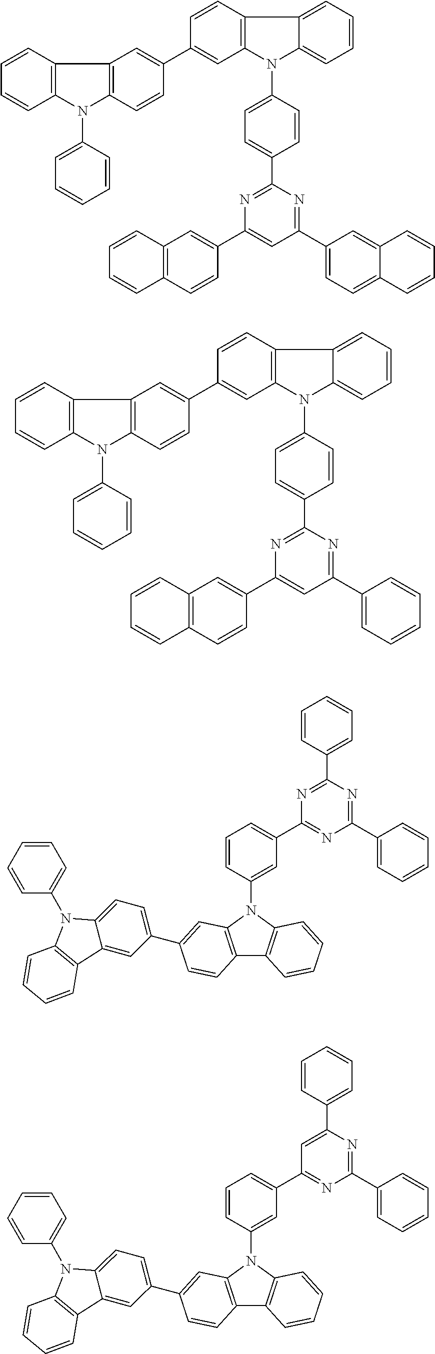

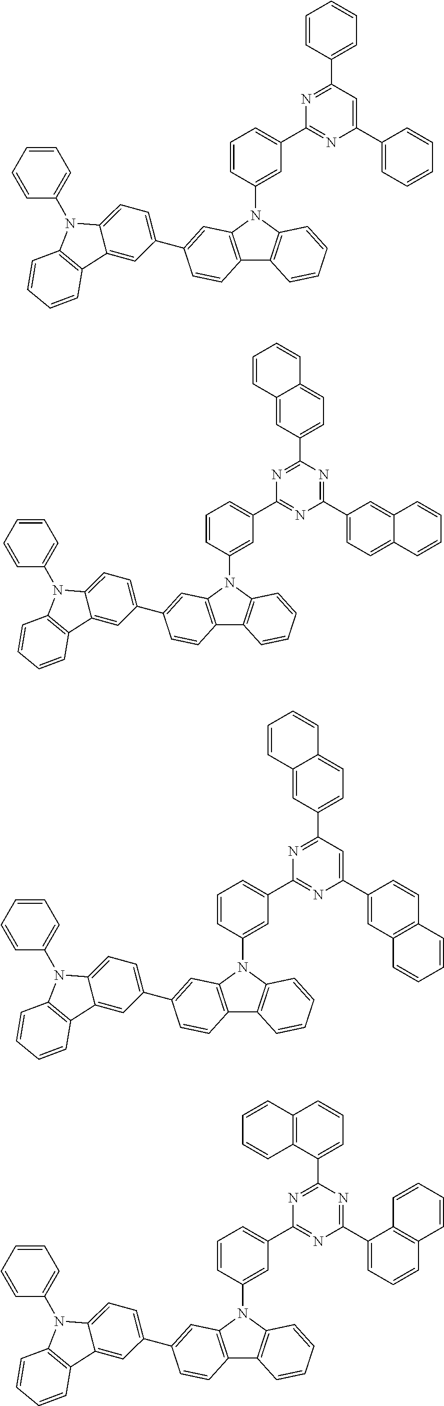 [SMILES: c1ccc(-c2cc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-n2c3ccccc3c3cc(-c4ccc5c6ccccc6n(-c6cccc(-c7nc(-c8ccc9ccccc9c8)nc(-c8ccc9ccccc9c8)n7)c6)c5c4)ccc32)cc1 ; c1ccc(-n2c3ccccc3c3cc(-c4ccc5c6ccccc6n(-c6cccc(-c7nc(-c8ccc9ccccc9c8)cc(-c8ccc9ccccc9c8)n7)c6)c5c4)ccc32)cc1 ; c1ccc(-n2c3ccccc3c3cc(-c4ccc5c6ccccc6n(-c6cccc(-c7nc(-c8cccc9ccccc89)nc(-c8cccc9ccccc89)n7)c6)c5c4)ccc32)cc1]

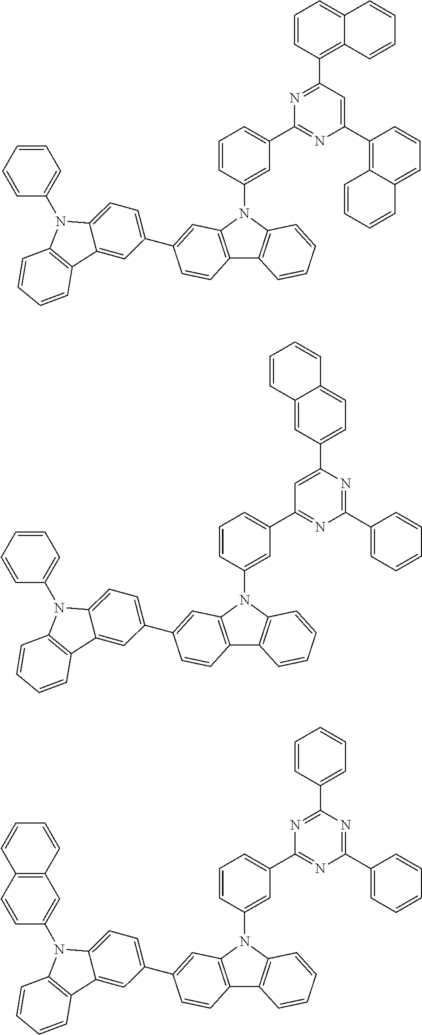 [SMILES: c1ccc(-n2c3ccccc3c3cc(-c4ccc5c6ccccc6n(-c6cccc(-c7nc(-c8cccc9ccccc89)cc(-c8cccc9ccccc89)n7)c6)c5c4)ccc32)cc1 ; c1ccc(-c2nc(-c3cccc(-n4c5ccccc5c5ccc(-c6ccc7c(c6)c6ccccc6n7-c6ccccc6)cc54)c3)cc(-c3ccc4ccccc4c3)n2)cc1 ; c1ccc(-c2nc(-c3ccccc3)nc(-c3cccc(-n4c5ccccc5c5ccc(-c6ccc7c(c6)c6ccccc6n7-c6ccc7ccccc7c6)cc54)c3)n2)cc1]

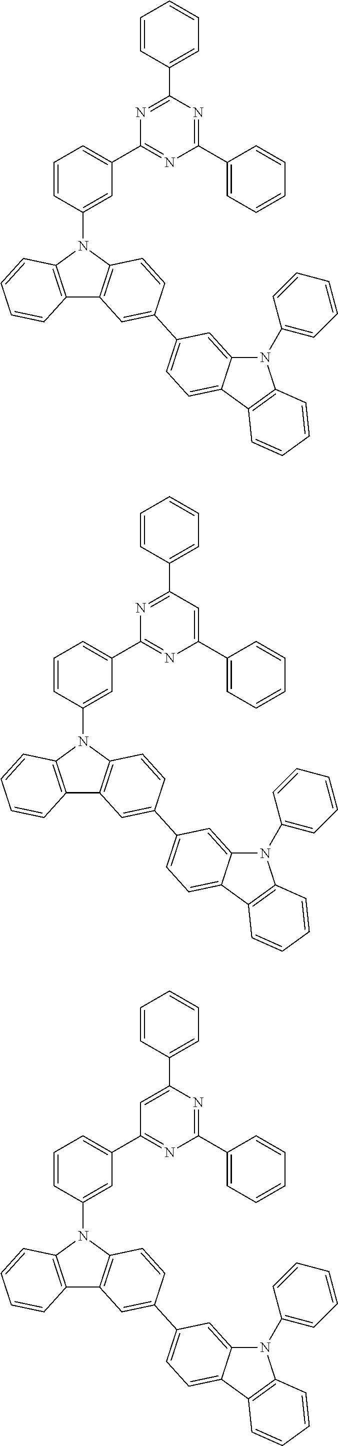 [SMILES: c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2cc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2cc(-c3cccc(-n4c5ccccc5c5cc(-c6ccc7c8ccccc8n(-c8ccccc8)c7c6)ccc54)c3)nc(-c3ccccc3)n2)cc1]

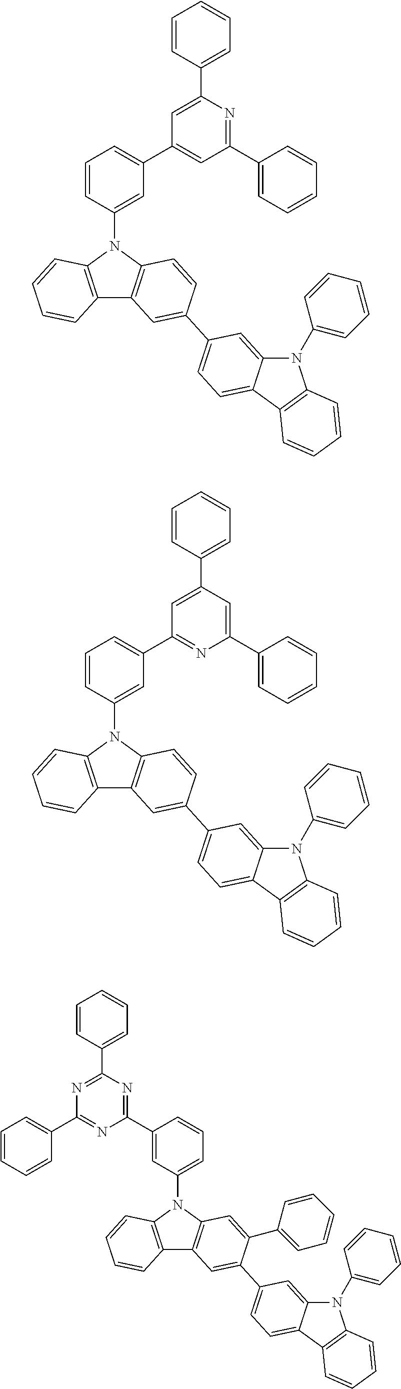 [SMILES: c1ccc(-c2cc(-c3cccc(-n4c5ccccc5c5cc(-c6ccc7c8ccccc8n(-c8ccccc8)c7c6)ccc54)c3)cc(-c3ccccc3)n2)cc1 ; c1ccc(-c2cc(-c3ccccc3)nc(-c3cccc(-n4c5ccccc5c5cc(-c6ccc7c8ccccc8n(-c8ccccc8)c7c6)ccc54)c3)c2)cc1 ; c1ccc(-c2nc(-c3ccccc3)nc(-c3cccc(-n4c5ccccc5c5cc(-c6ccc7c8ccccc8n(-c8ccccc8)c7c6)c(-c6ccccc6)cc54)c3)n2)cc1]

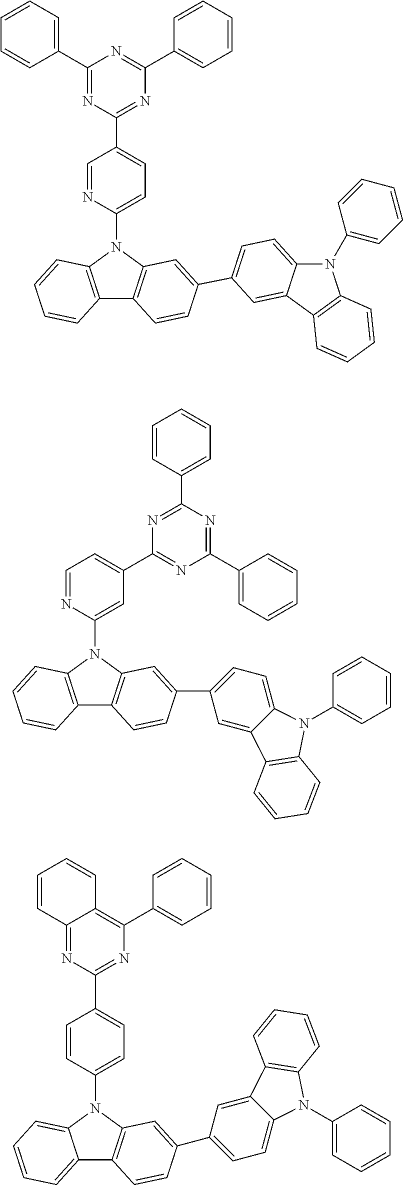

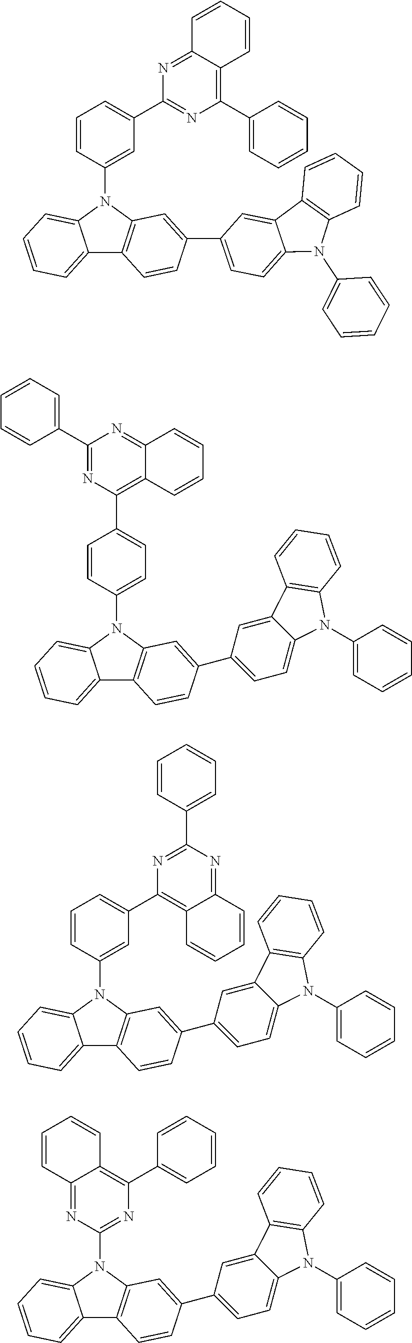 [SMILES: c1ccc(-c2nc(-c3cccc(-n4c5ccccc5c5ccc(-c6ccc7c(c6)c6ccccc6n7-c6ccccc6)cc54)c3)nc3ccccc23)cc1 ; c1ccc(-c2nc(-c3ccc(-n4c5ccccc5c5ccc(-c6ccc7c(c6)c6ccccc6n7-c6ccccc6)cc54)cc3)c3ccccc3n2)cc1 ; c1ccc(-c2nc(-c3cccc(-n4c5ccccc5c5ccc(-c6ccc7c(c6)c6ccccc6n7-c6ccccc6)cc54)c3)c3ccccc3n2)cc1 ; c1ccc(-c2nc(-n3c4ccccc4c4ccc(-c5ccc6c(c5)c5ccccc5n6-c5ccccc5)cc43)nc3ccccc23)cc1]

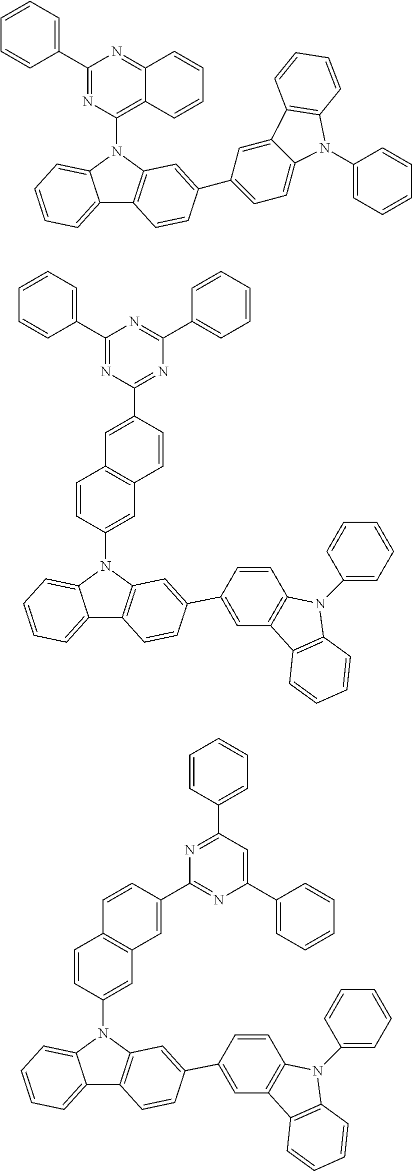

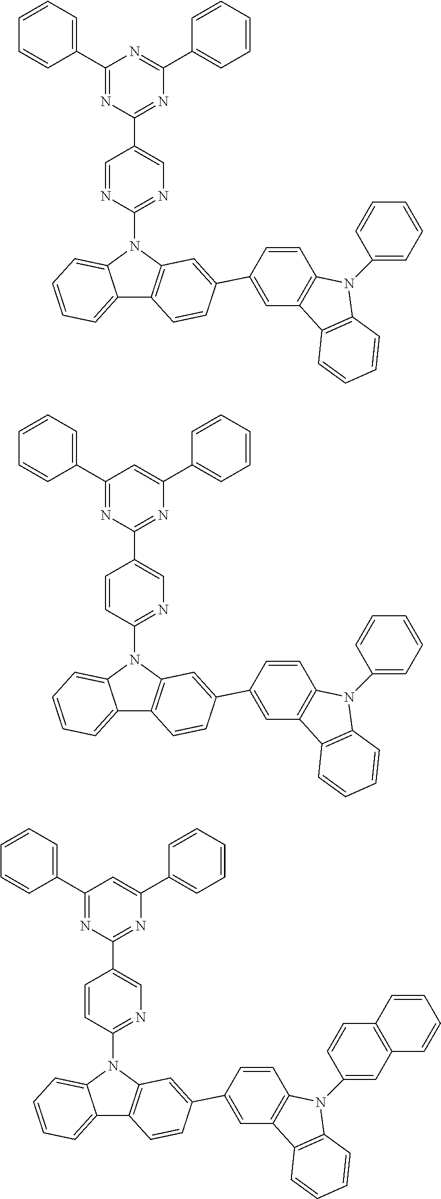 [SMILES: c1ccc(-c2nc(-c3ccccc3)nc(-c3cnc(-n4c5ccccc5c5ccc(-c6ccc7c(c6)c6ccccc6n7-c6ccccc6)cc54)nc3)n2)cc1 ; c1ccc(-c2cc(-c3ccccc3)nc(-c3ccc(-n4c5ccccc5c5ccc(-c6ccc7c(c6)c6ccccc6n7-c6ccccc6)cc54)nc3)n2)cc1 ; c1ccc(-c2cc(-c3ccccc3)nc(-c3ccc(-n4c5ccccc5c5ccc(-c6ccc7c(c6)c6ccccc6n7-c6ccc7ccccc7c6)cc54)nc3)n2)cc1]

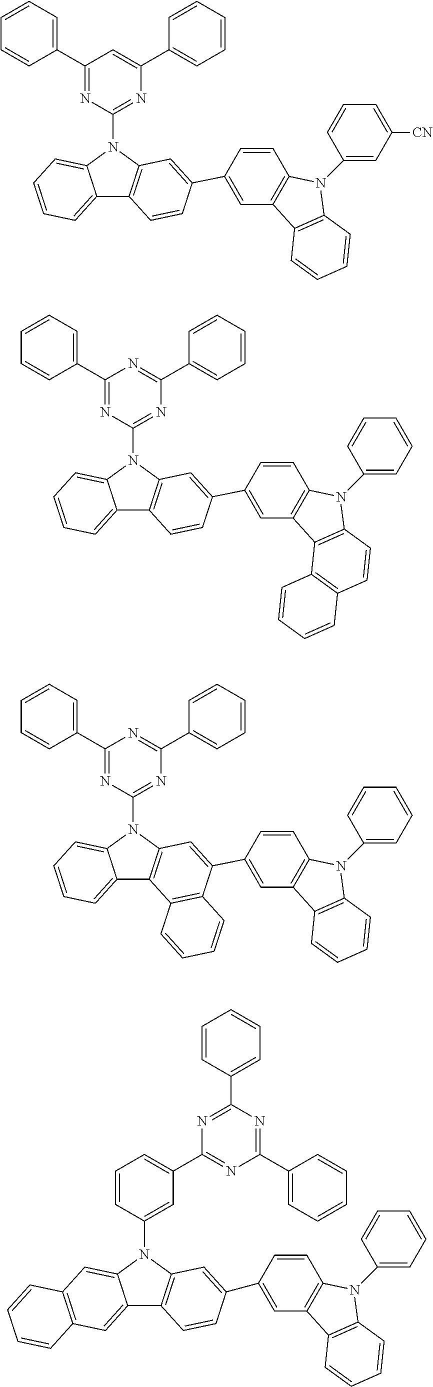 [SMILES: N#Cc1cccc(-n2c3ccccc3c3cc(-c4ccc5c6ccccc6n(-c6nc(-c7ccccc7)cc(-c7ccccc7)n6)c5c4)ccc32)c1 ; c1ccc(-c2nc(-c3ccccc3)nc(-n3c4ccccc4c4ccc(-c5ccc6c(c5)c5c7ccccc7ccc5n6-c5ccccc5)cc43)n2)cc1 ; c1ccc(-c2nc(-c3ccccc3)nc(-n3c4ccccc4c4c5ccccc5c(-c5ccc6c(c5)c5ccccc5n6-c5ccccc5)cc43)n2)cc1 ; c1ccc(-c2nc(-c3ccccc3)nc(-c3cccc(-n4c5cc(-c6ccc7c(c6)c6ccccc6n7-c6ccccc6)ccc5c5cc6ccccc6cc54)c3)n2)cc1]

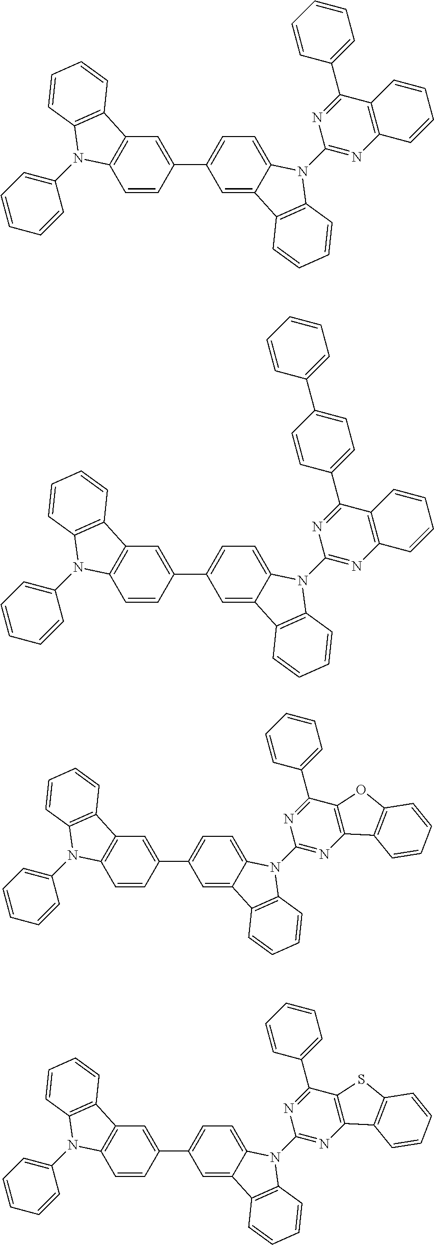

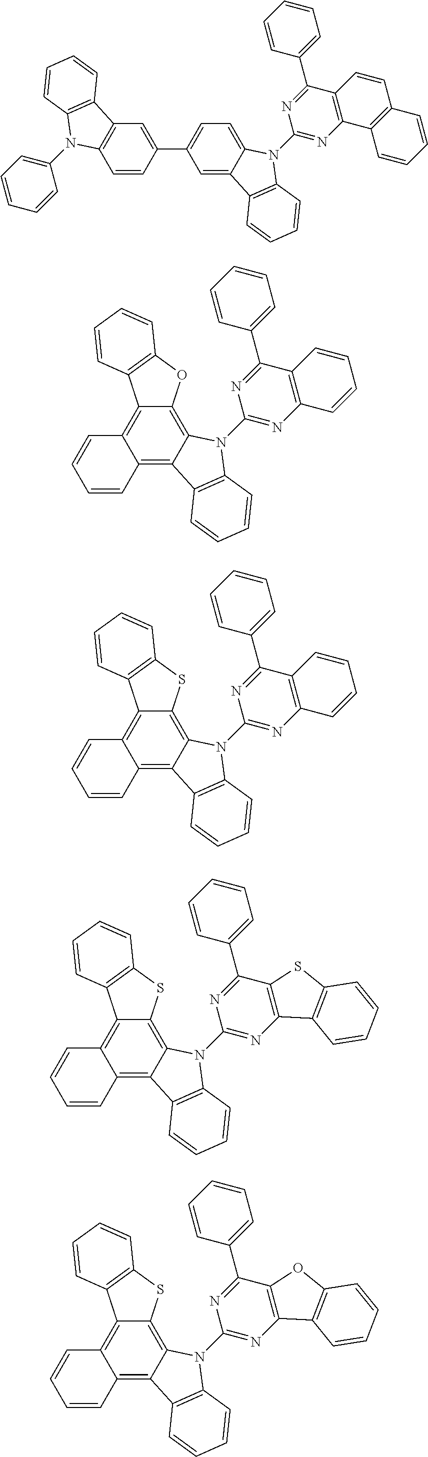

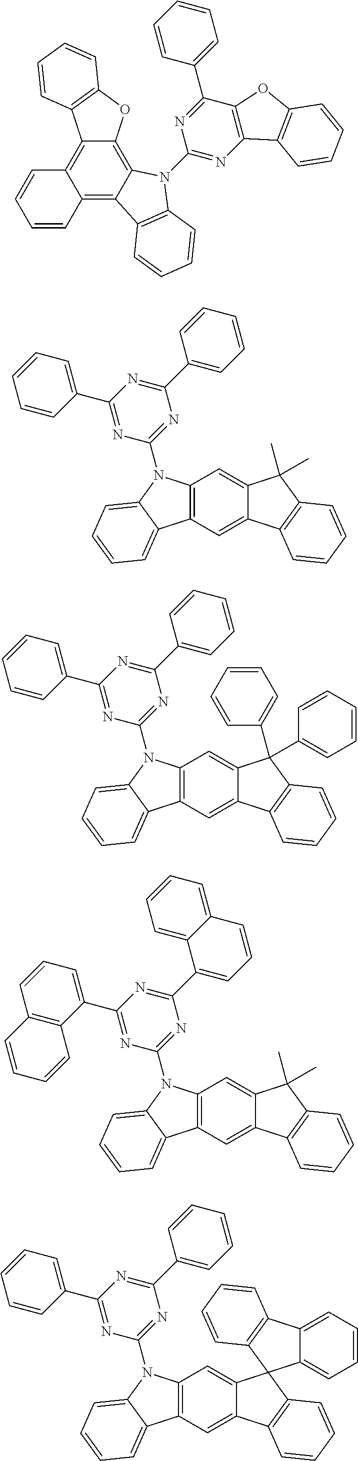

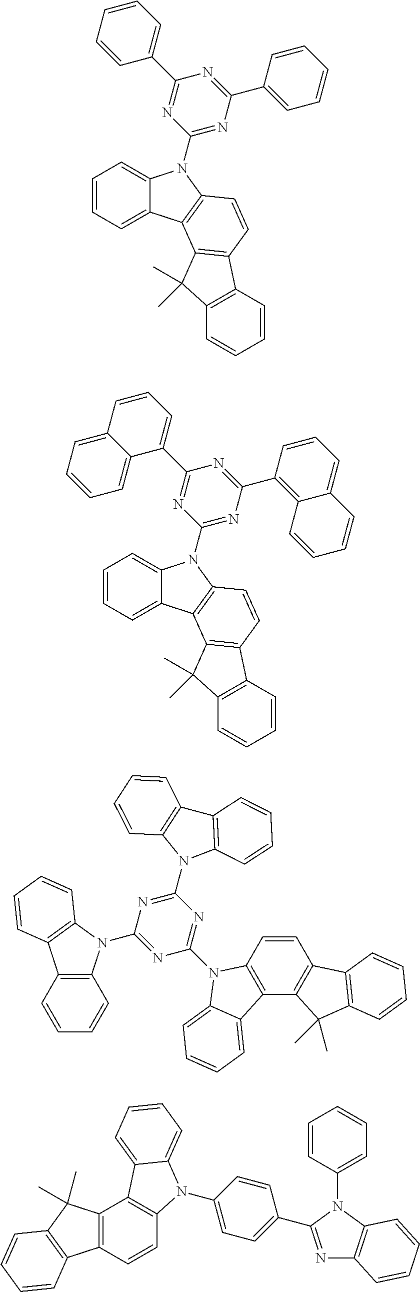

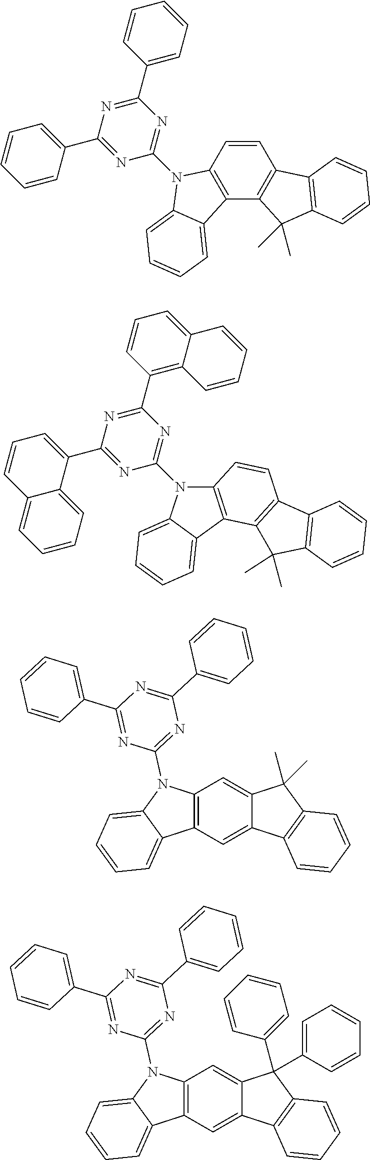

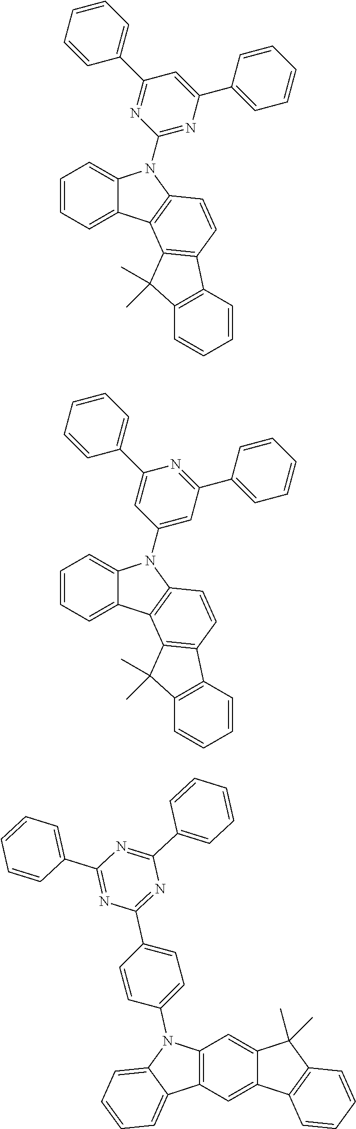

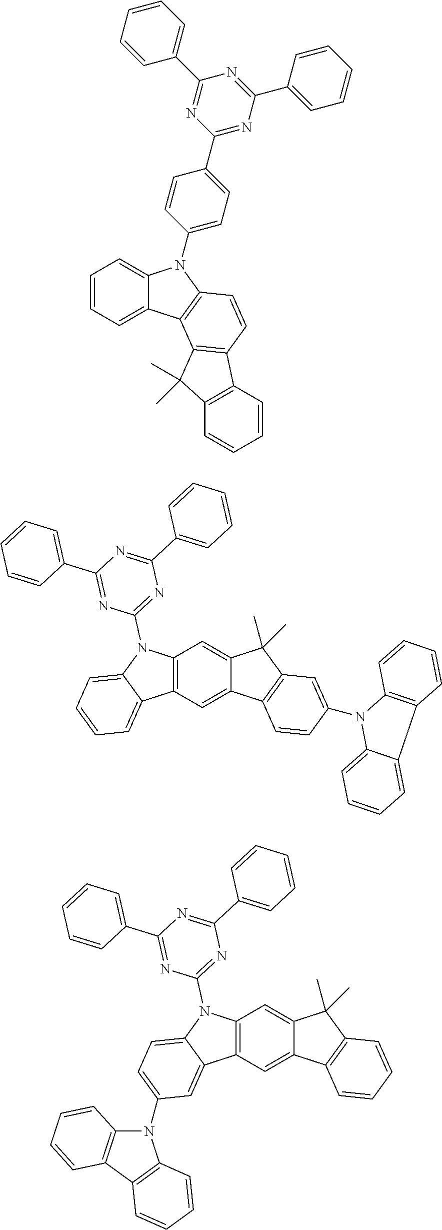

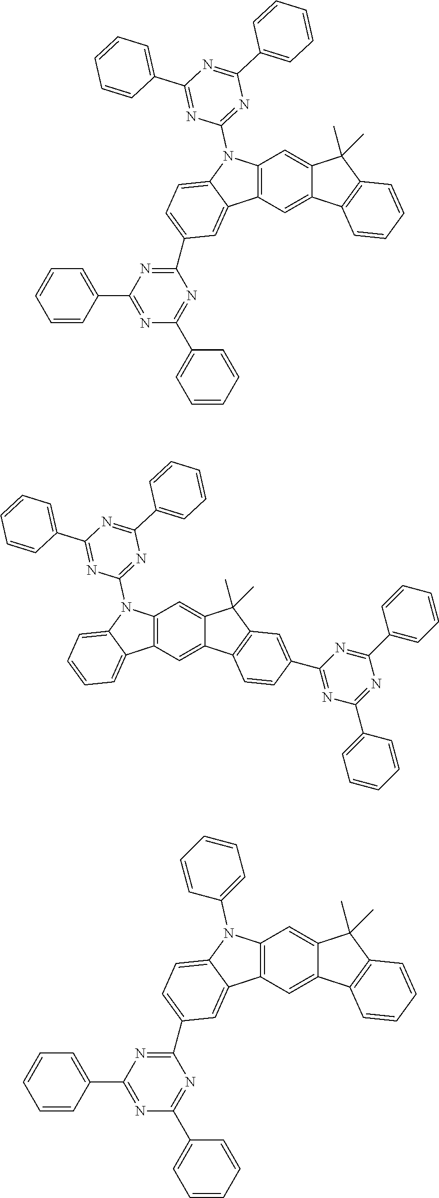 [SMILES: CC1(C)c2ccccc2-c2cc3c4cc(-c5nc(-c6ccccc6)nc(-c6ccccc6)n5)ccc4n(-c4nc(-c5ccccc5)nc(-c5ccccc5)n4)c3cc21 ; CC1(C)c2cc(-c3nc(-c4ccccc4)nc(-c4ccccc4)n3)ccc2-c2cc3c4ccccc4n(-c4nc(-c5ccccc5)nc(-c5ccccc5)n4)c3cc21 ; CC1(C)c2ccccc2-c2cc3c4cc(-c5nc(-c6ccccc6)nc(-c6ccccc6)n5)ccc4n(-c4ccccc4)c3cc21]

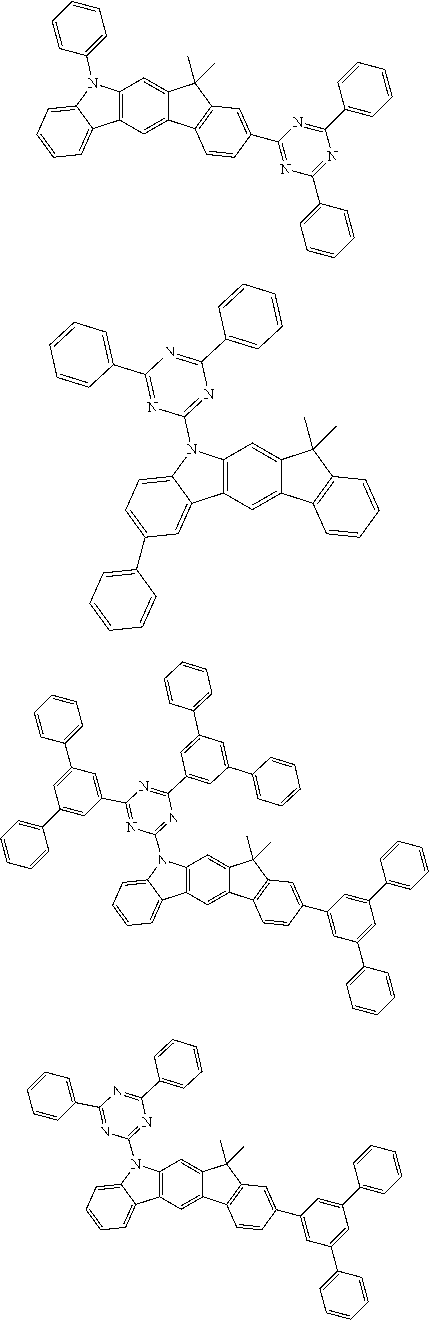 [SMILES: CC1(C)c2cc(-c3nc(-c4ccccc4)nc(-c4ccccc4)n3)ccc2-c2cc3c4ccccc4n(-c4ccccc4)c3cc21 ; CC1(C)c2ccccc2-c2cc3c4cc(-c5ccccc5)ccc4n(-c4nc(-c5ccccc5)nc(-c5ccccc5)n4)c3cc21 ; CC1(C)c2cc(-c3cc(-c4ccccc4)cc(-c4ccccc4)c3)ccc2-c2cc3c4ccccc4n(-c4nc(-c5cc(-c6ccccc6)cc(-c6ccccc6)c5)nc(-c5cc(-c6ccccc6)cc(-c6ccccc6)c5)n4)c3cc21 ; CC1(C)c2cc(-c3cc(-c4ccccc4)cc(-c4ccccc4)c3)ccc2-c2cc3c4ccccc4n(-c4nc(-c5ccccc5)nc(-c5ccccc5)n4)c3cc21]

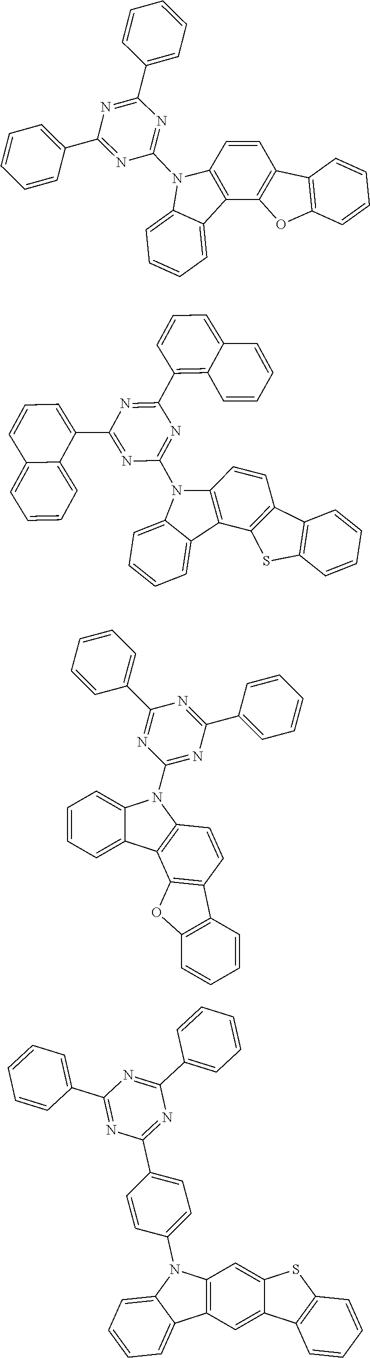

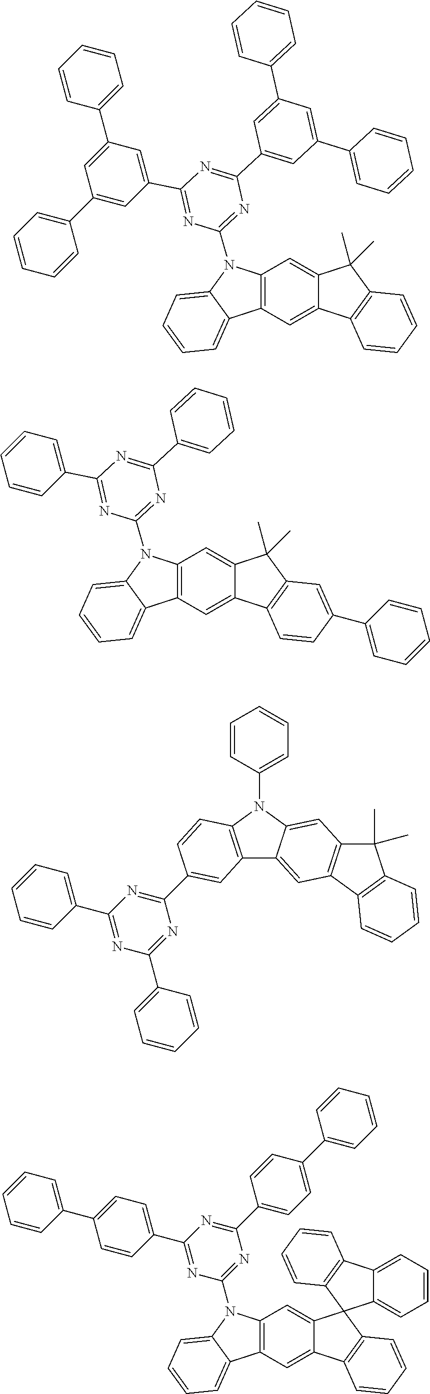

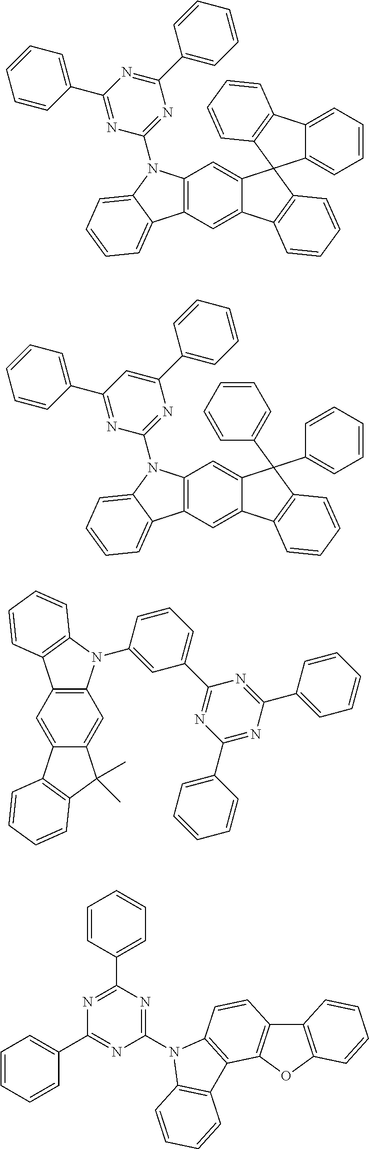 [SMILES: c1ccc(-c2nc(-c3ccccc3)nc(-n3c4ccccc4c4cc5c(cc43)C3(c4ccccc4-c4ccccc43)c3ccccc3-5)n2)cc1 ; c1ccc(-c2cc(-c3ccccc3)nc(-n3c4ccccc4c4cc5c(cc43)C(c3ccccc3)(c3ccccc3)c3ccccc3-5)n2)cc1 ; CC1(C)c2ccccc2-c2cc3c4ccccc4n(-c4cccc(-c5nc(-c6ccccc6)nc(-c6ccccc6)n5)c4)c3cc21 ; c1ccc(-c2nc(-c3ccccc3)nc(-n3c4ccccc4c4c5oc6ccccc6c5ccc43)n2)cc1]

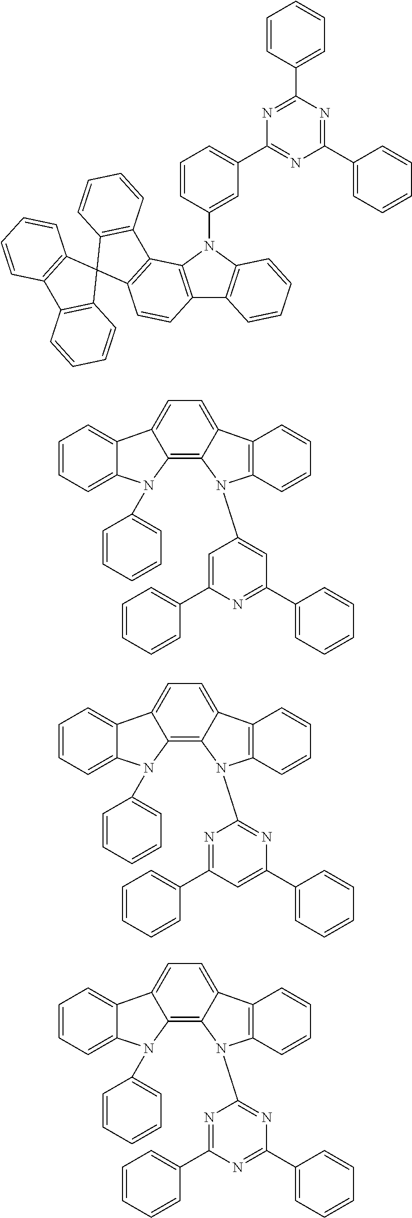

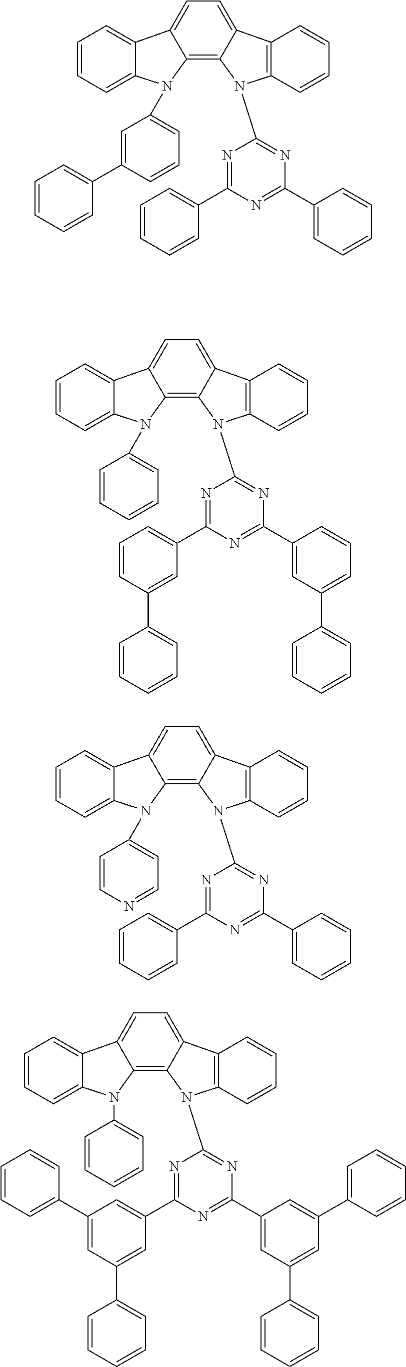

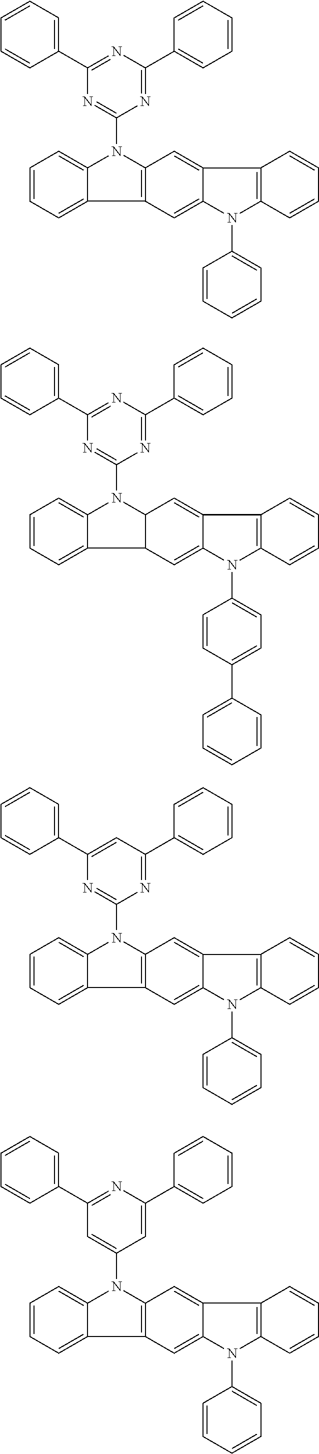

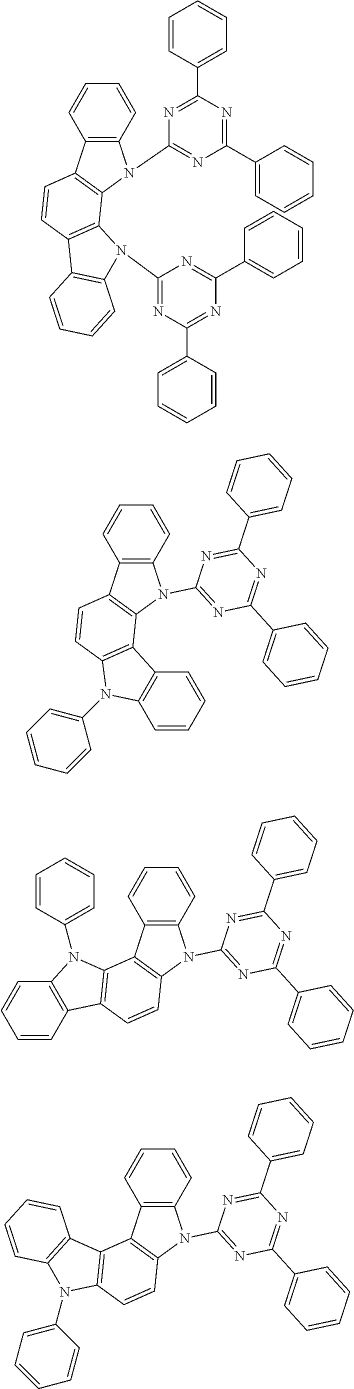

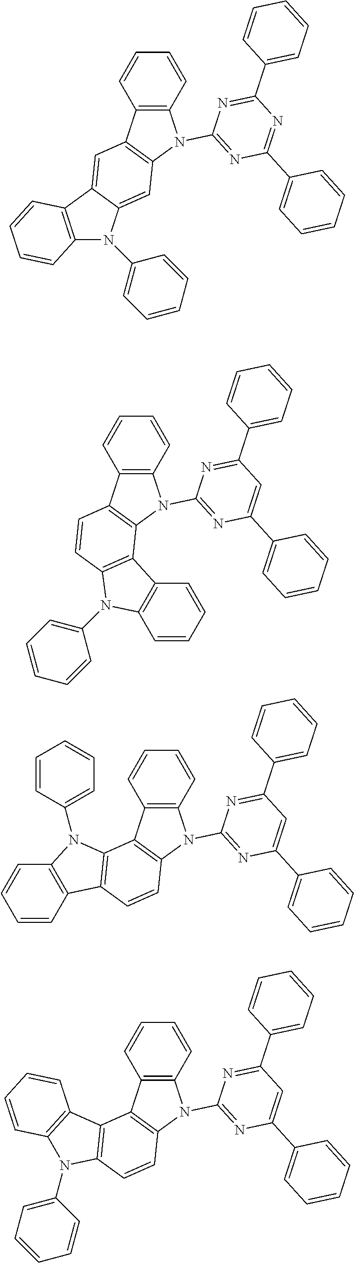

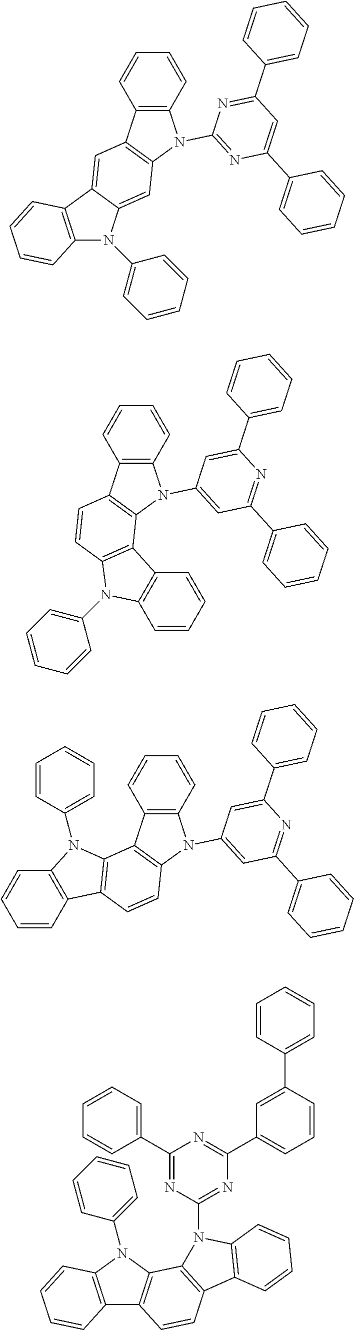 [SMILES: c1ccc(-c2cc(-c3ccccc3)nc(-n3c4ccccc4c4cc5c6ccccc6n(-c6ccccc6)c5cc43)n2)cc1 ; c1ccc(-c2cc(-n3c4ccccc4c4ccc5c(c6ccccc6n5-c5ccccc5)c43)cc(-c3ccccc3)n2)cc1 ; c1ccc(-c2cc(-n3c4ccccc4c4c3ccc3c5ccccc5n(-c5ccccc5)c34)cc(-c3ccccc3)n2)cc1 ; c1ccc(-c2cccc(-c3nc(-c4ccccc4)nc(-n4c5ccccc5c5ccc6c7ccccc7n(-c7ccccc7)c6c54)n3)c2)cc1]

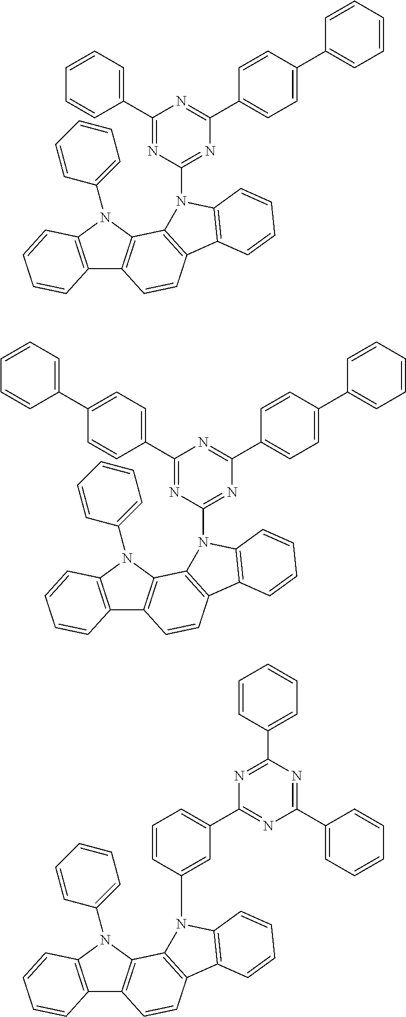 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1 ; c1ccc(-c2nc(-c3ccccc3)nc(-c3cccc(-n4c5ccccc5c5ccc6c7ccccc7n(-c7ccccc7)c6c54)c3)n2)cc1]

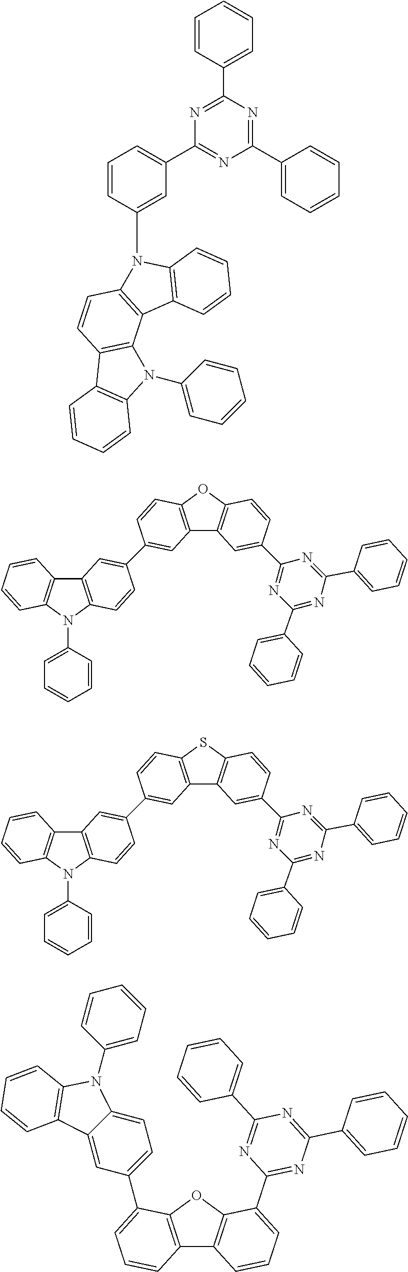 [SMILES: c1ccc(-c2nc(-c3ccccc3)nc(-c3cccc(-n4c5ccccc5c5c4ccc4c6ccccc6n(-c6ccccc6)c45)c3)n2)cc1 ; c1ccc(-c2nc(-c3ccccc3)nc(-c3ccc4oc5ccc(-c6ccc7c(c6)c6ccccc6n7-c6ccccc6)cc5c4c3)n2)cc1 ; c1ccc(-c2nc(-c3ccccc3)nc(-c3ccc4sc5ccc(-c6ccc7c(c6)c6ccccc6n7-c6ccccc6)cc5c4c3)n2)cc1 ; c1ccc(-c2nc(-c3ccccc3)nc(-c3cccc4c3oc3c(-c5ccc6c(c5)c5ccccc5n6-c5ccccc5)cccc34)n2)cc1]

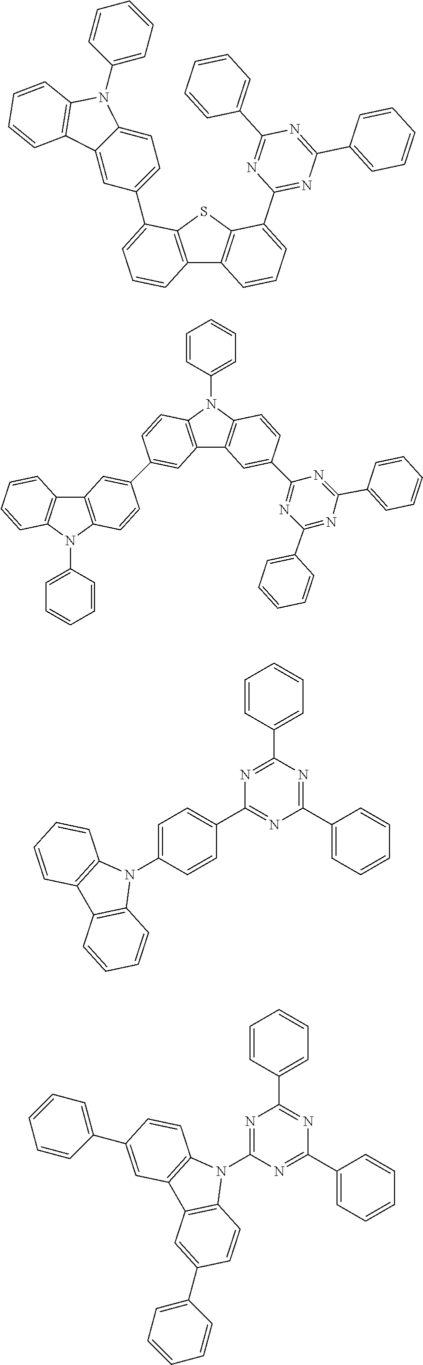

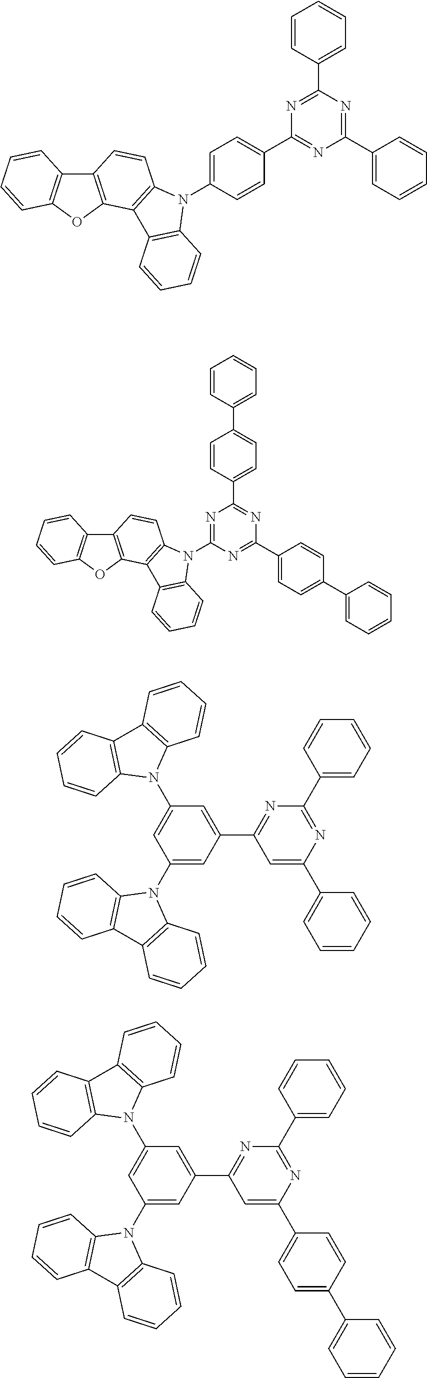

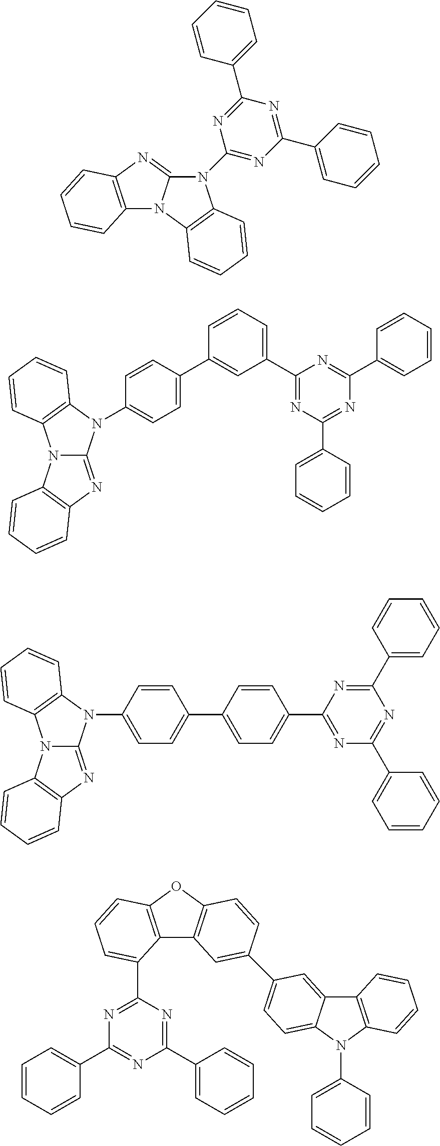

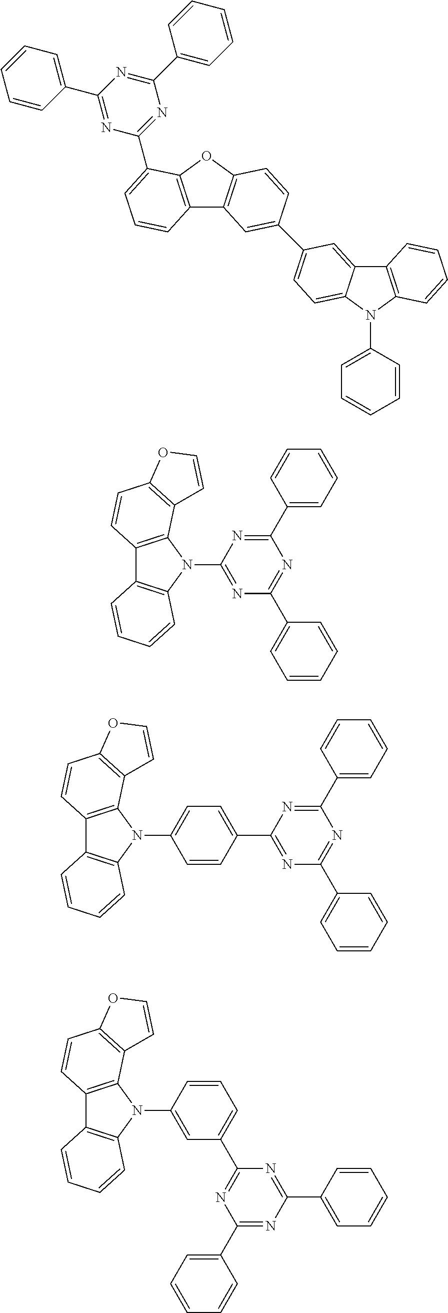

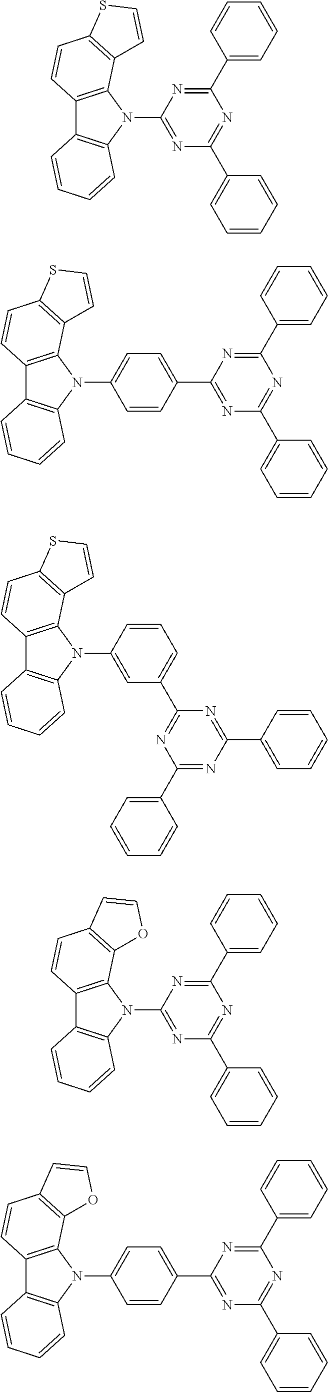

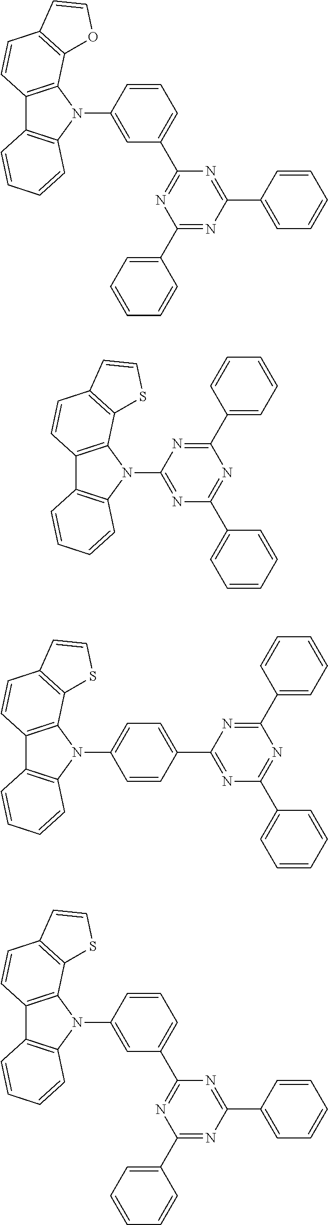 [SMILES: c1ccc(-c2nc(-c3ccccc3)nc(-c3cccc(-n4c5ccccc5c5ccc6ccoc6c54)c3)n2)cc1 ; c1ccc(-c2nc(-c3ccccc3)nc(-n3c4ccccc4c4ccc5ccsc5c43)n2)cc1 ; c1ccc(-c2nc(-c3ccccc3)nc(-c3ccc(-n4c5ccccc5c5ccc6ccsc6c54)cc3)n2)cc1 ; c1ccc(-c2nc(-c3ccccc3)nc(-c3cccc(-n4c5ccccc5c5ccc6ccsc6c54)c3)n2)cc1]

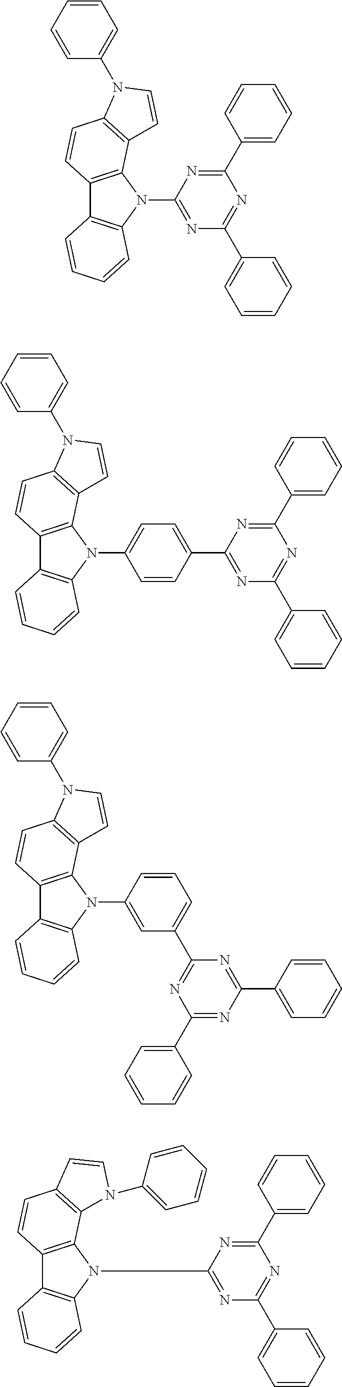

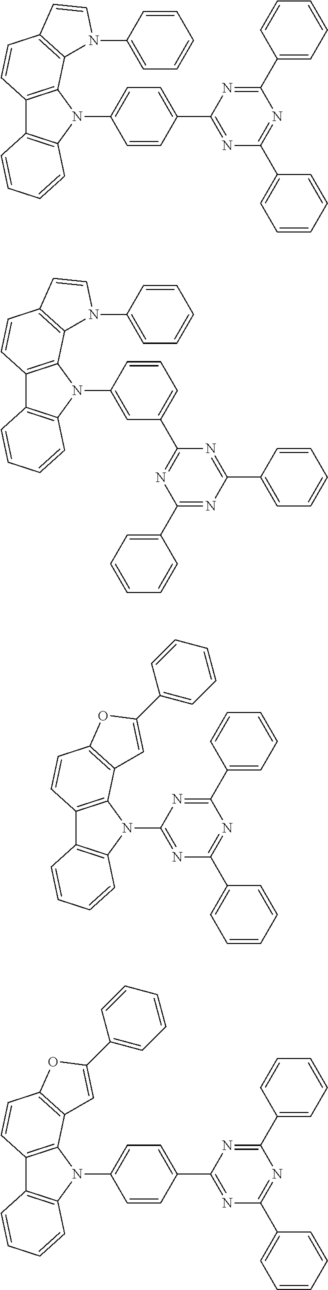 [SMILES: c1ccc(-c2nc(-c3ccccc3)nc(-c3ccc(-n4c5ccccc5c5ccc6ccn(-c7ccccc7)c6c54)cc3)n2)cc1 ; c1ccc(-c2nc(-c3ccccc3)nc(-c3cccc(-n4c5ccccc5c5ccc6ccn(-c7ccccc7)c6c54)c3)n2)cc1 ; c1ccc(-c2nc(-c3ccccc3)nc(-n3c4ccccc4c4ccc5oc(-c6ccccc6)cc5c43)n2)cc1 ; c1ccc(-c2nc(-c3ccccc3)nc(-c3ccc(-n4c5ccccc5c5ccc6oc(-c7ccccc7)cc6c54)cc3)n2)cc1]

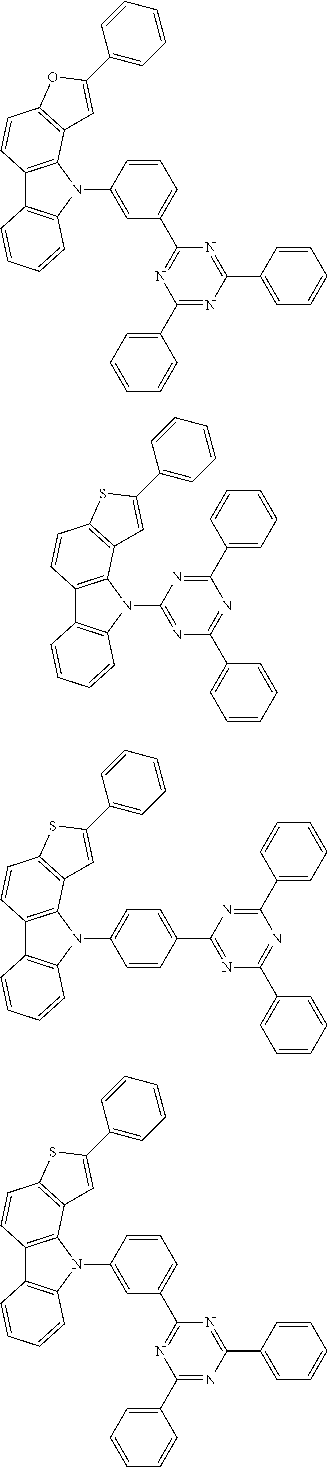

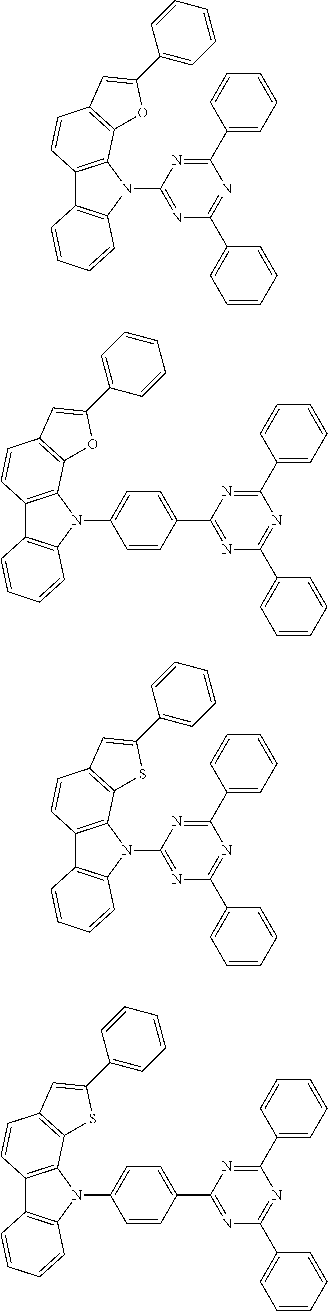 [SMILES: c1ccc(-c2nc(-c3ccccc3)nc(-n3c4ccccc4c4ccc5cc(-c6ccccc6)oc5c43)n2)cc1 ; c1ccc(-c2nc(-c3ccccc3)nc(-c3ccc(-n4c5ccccc5c5ccc6cc(-c7ccccc7)oc6c54)cc3)n2)cc1 ; c1ccc(-c2nc(-c3ccccc3)nc(-n3c4ccccc4c4ccc5cc(-c6ccccc6)sc5c43)n2)cc1 ; c1ccc(-c2nc(-c3ccccc3)nc(-c3ccc(-n4c5ccccc5c5ccc6cc(-c7ccccc7)sc6c54)cc3)n2)cc1]

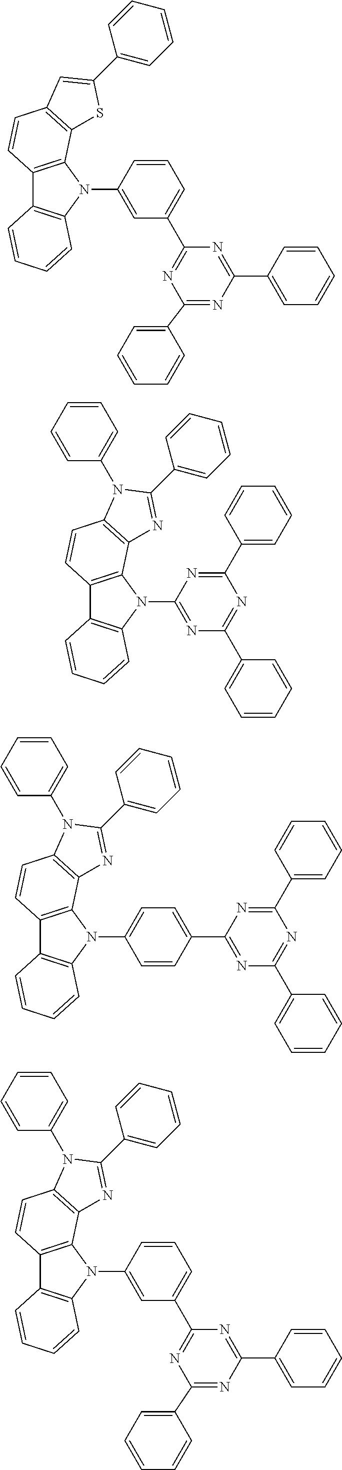

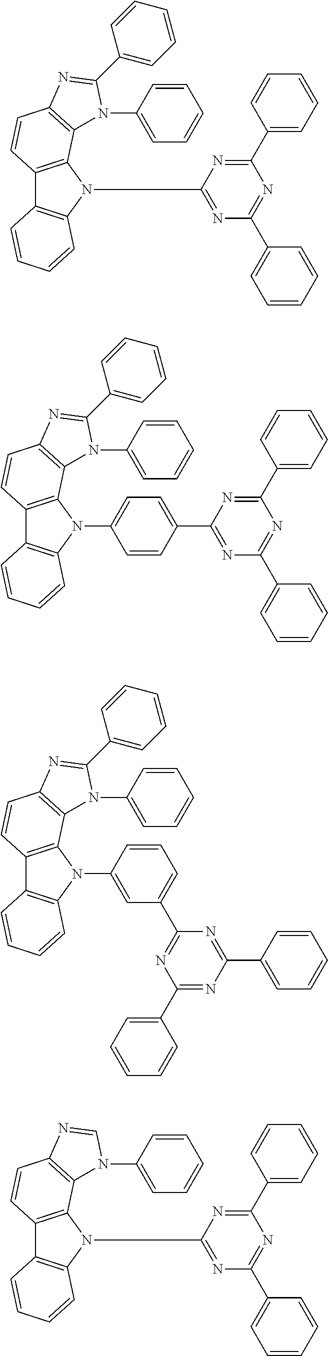 [SMILES: c1ccc(-c2nc(-c3ccccc3)nc(-n3c4ccccc4c4ccc5nc(-c6ccccc6)n(-c6ccccc6)c5c43)n2)cc1 ; c1ccc(-c2nc(-c3ccccc3)nc(-c3ccc(-n4c5ccccc5c5ccc6nc(-c7ccccc7)n(-c7ccccc7)c6c54)cc3)n2)cc1 ; c1ccc(-c2nc(-c3ccccc3)nc(-c3cccc(-n4c5ccccc5c5ccc6nc(-c7ccccc7)n(-c7ccccc7)c6c54)c3)n2)cc1 ; c1ccc(-c2nc(-c3ccccc3)nc(-n3c4ccccc4c4ccc5ncn(-c6ccccc6)c5c43)n2)cc1]

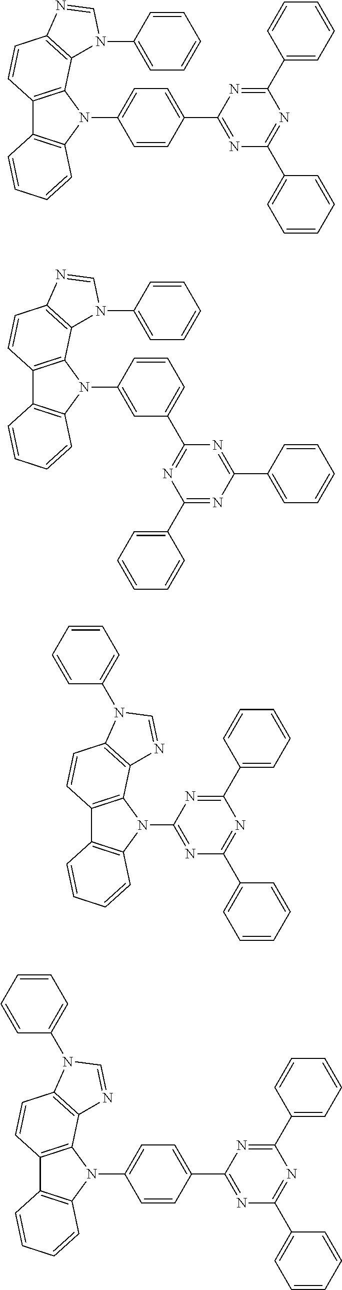 [SMILES: c1ccc(-c2nc(-c3ccccc3)nc(-c3ccc(-n4c5ccccc5c5ccc6ncn(-c7ccccc7)c6c54)cc3)n2)cc1 ; c1ccc(-c2nc(-c3ccccc3)nc(-c3cccc(-n4c5ccccc5c5ccc6ncn(-c7ccccc7)c6c54)c3)n2)cc1 ; c1ccc(-c2nc(-c3ccccc3)nc(-n3c4ccccc4c4ccc5c(ncn5-c5ccccc5)c43)n2)cc1 ; c1ccc(-c2nc(-c3ccccc3)nc(-c3ccc(-n4c5ccccc5c5ccc6c(ncn6-c6ccccc6)c54)cc3)n2)cc1]

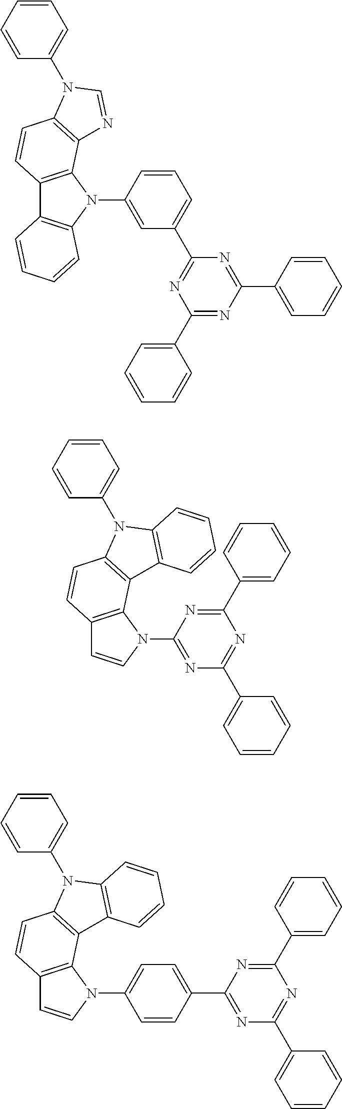 [SMILES: c1ccc(-c2nc(-c3ccccc3)nc(-c3cccc(-n4c5ccccc5c5ccc6c(ncn6-c6ccccc6)c54)c3)n2)cc1 ; c1ccc(-c2nc(-c3ccccc3)nc(-n3ccc4ccc5c(c6ccccc6n5-c5ccccc5)c43)n2)cc1 ; c1ccc(-c2nc(-c3ccccc3)nc(-c3ccc(-n4ccc5ccc6c(c7ccccc7n6-c6ccccc6)c54)cc3)n2)cc1]

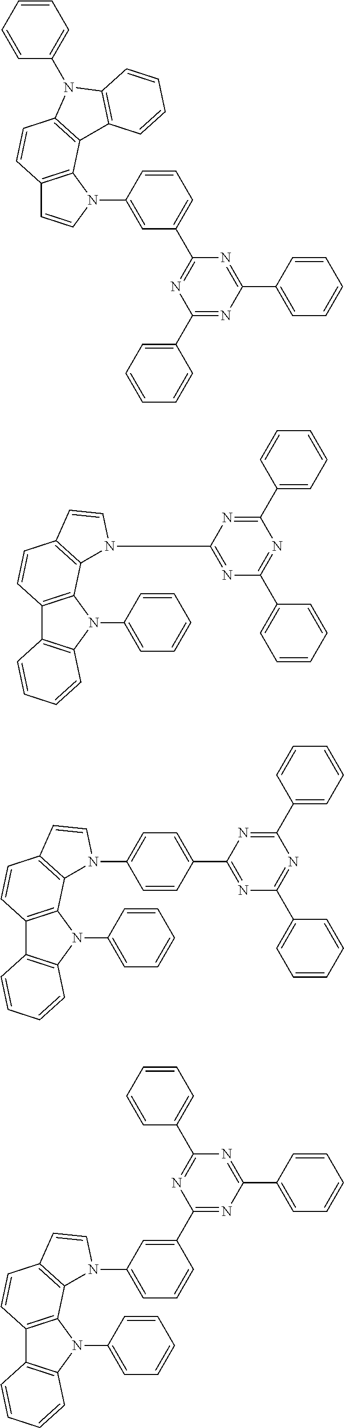 [SMILES: c1ccc(-c2nc(-c3ccccc3)nc(-c3cccc(-n4ccc5ccc6c(c7ccccc7n6-c6ccccc6)c54)c3)n2)cc1 ; c1ccc(-c2nc(-c3ccccc3)nc(-n3ccc4ccc5c6ccccc6n(-c6ccccc6)c5c43)n2)cc1 ; c1ccc(-c2nc(-c3ccccc3)nc(-c3ccc(-n4ccc5ccc6c7ccccc7n(-c7ccccc7)c6c54)cc3)n2)cc1 ; c1ccc(-c2nc(-c3ccccc3)nc(-c3cccc(-n4ccc5ccc6c7ccccc7n(-c7ccccc7)c6c54)c3)n2)cc1]

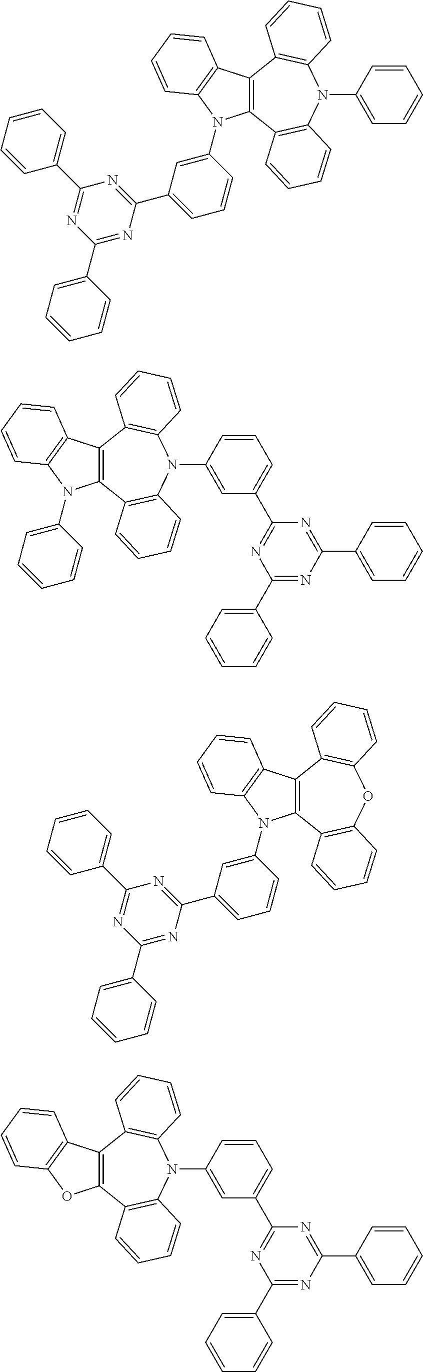

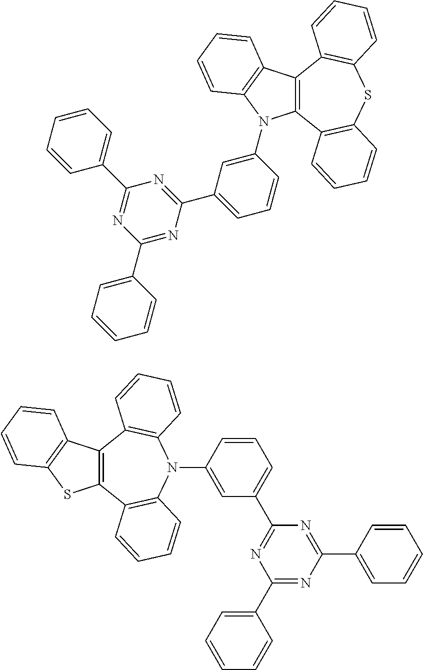

In one embodiment of the present invention, the compounding ratio of the compound represented by the formula (1) and the compound represented by the formula (2) is not particularly limited and is appropriately determined in accordance with the effects to be achieved. The compounding ratio (ratio by mass) of the compound represented by the formula (1) to the compound represented by the formula (2) is ordinarily in a range of 1:99 to 99:1, and preferably 10:90 to 90:10.

In the organic EL device according to one aspect of the present invention, the emitting layer may further contain an emitting material.

In one embodiment of the present invention, it is preferable that the emitting layer comprise a phosphorescent emitting material as an emitting material and that the phosphorescent emitting material be an ortho-metalated complex of a metal atom selected from iridium (Ir), osmium (Os) and platinum (Pt). Preferred phosphorescent emitting materials are described later.

The organic EL device may further comprise a hole transporting layer between the anode and the emitting layer and may further comprise an electron transporting layer between the cathode and the emitting layer.

The above-mentioned fluorescent emitting materials, phosphorescent emitting materials, and materials used in the hole transporting layer and electron transporting layer are described later.

Each layer of the organic EL device according to one aspect of the present invention is formed by a dry film-formation method such as vacuum deposition, sputtering, plasma, or ion plating, or a wet film-formation method such as spin coating, dipping, or flow coating. The film thickness is not particularly limited but it needs to be appropriately determined. If the film is too thick, a high voltage needs to be applied to obtain a given optical output, whereby efficiency could be deteriorated. If the film is too thin, pinholes and the like could be formed such that a sufficient luminance could not be achieved even by applying an electric field. Ordinarily, the suitable film thickness is in a range of 5 nm to 10 μm, and more preferably 10 nm to 0.2 μm.

Materials and the like of the components constituting the organic EL device are described below.

(Substrate)

A substrate is used as a support of an emitting device. As a substrate, glass, quartz, or plastics may be used, for example. A flexible substrate is also applicable. A flexible substrate is a bendable (i.e., flexible) substrate and is exemplified by plastic substrates made of polycarbonate, polyvinyl chloride, or the like.

(Anode)

In an anode formed on the substrate, a metal, an alloy, or an electroconductive compound that has a high work function (specifically 4.0 eV or higher), a mixture thereof, or the like is preferably used. Specifically, indium oxide-tin oxide (ITO; Indium Tin Oxide), indium oxide-tin oxide containing silicon or silicon oxide, indium oxide-zinc oxide, tungsten oxide, indium oxide containing zinc oxide, and graphene are mentioned as examples. In addition, gold (Au), platinum (Pt), and nitrides of metal materials (such as titanium nitride) are mentioned.

(Hole Injection Layer)

A hole injection layer is a layer containing a material having a high hole-injecting property. As the material having a high hole-injecting property, a molybdenum oxide, a titanium oxide, a vanadium oxide, a rhenium oxide, a ruthenium oxide, a chromium oxide, a zirconium oxide, a hafnium oxide, a tantalum oxide, a silver oxide, a tungsten oxide, a manganese oxide, an aromatic amine compound, or a high-molecular compound (such as an oligomer, a dendrimer, or a polymer) may be used.

(Hole Transporting Layer)

A hole transporting layer is a layer containing a material having a high hole-transporting property. In the hole transporting layer, an aromatic amine compound, a carbazole derivative, an anthracene derivative, or the like may be used. A high-molecular compound such as poly(N-vinylcarbazole) (abbreviated as PVK) or poly(4-vinyltriphenylamine) (abbreviated as PVTPA) may also be used. Any other materials may be used as long as said materials have hole-transporting properties higher than electron-transporting properties. The layer containing a material having a high hole-transporting property may be not only a single layer but also two or more laminated layers each composed of any one of the above materials.

(Guest Material of Emitting Layer)

An emitting layer is a layer containing a material having a high emitting property, and various types of materials can be used. For example, as a material having a high emitting property, a fluorescent compound that emits fluorescence or a phosphorescent compound that emits phosphorescence may be used. A fluorescent compound is a compound that can emit light from the singlet excited state, and a phosphorescent compound is a compound that can emit light from the triplet excited state. These compounds may be called dopants or dopant materials.

As a blue fluorescent emitting material that is usable in the light emitting layer, a pyrene derivative, a styrylamine derivative, a chrysene derivative, a fluoranthene derivative, a fluorene derivative, a diamine derivative, a triarylamine derivative or the like may be used. Specific examples thereof include N,N′-bis[4-(9H-carbazol-9-yl)phenyl]-N,N′-diphenylstilbene-4,4′-diamine (abbreviated as YGA2S), 4-(9H-carbazol-9-yl)-4′-(10-phenyl-9-anthryl)triphenylamine (abbreviated as YGAPA), and 4-(10-phenyl-9-anthryl)-4′-(9-phenyl-9H-carbazol-3-yl)triphenylamine (abbreviated as PCBAPA).

As a green fluorescent emitting material that is usable in the emitting layer, an aromatic amine derivative may be used. Specific examples thereof include N-(9,10-diphenyl-2-anthryl)-N,9-diphenyl-9H-carbazole-3-amine (abbreviated as 2PCAPA), N-[9,10-bis(1,1′-biphenyl-2-yl)-2-anthryl]-N,9-diphenyl-9H-carbazole-3-amine (abbreviated as 2PCABPhA), N-(9,10-diphenyl-2-anthryl)-N,N′,N′-triphenyl-1,4-phenylenediamine (abbreviated as 2DPAPA), N-[9,10-bis(1,1′-biphenyl-2-yl)-2-anthryl]-N,N′,N′-triphenyl-1,4-phenylenediamine (abbreviated as 2DPABPhA), N-[9,10-bis(1,1′-biphenyl-2-yl)]-N-[4-(9H-carbazol-9-yl)phenyl]-N-phenylanthracene-2-amine (abbreviated as 2YGABPhA), and N,N,9-triphenylanthracene-9-amine (abbreviated as DPhAPhA).

As a red fluorescent emitting material that is usable in the emitting layer, a tetracene derivative or a diamine derivative may be used. Specific examples thereof include N,N,N′,N′-tetrakis(4-methylphenyl) tetracene-5,11-diamine (abbreviated as p-mPhTD), and 7,14-diphenyl-N,N,N′N′-tetrakis(4-methylphenyl)acenaphtho[1,2-a]fluoranthene-3,10-diamine (abbreviated as p-mPhAFD).

According to one aspect of the present invention, the fluorescent emitting material preferably contains at least one derivative selected from anthracene derivatives, fluoranthene derivatives, styrylamine derivatives, and arylamine derivatives.

As a blue phosphorescent emitting material that is usable in the emitting layer, a metal complex such as an iridium complex, an osmium complex, or a platinum complex may be used. Specific examples thereof include bis[2-(4′,6′-difluorophenyl)pyridinato-N,C2′]iridium(III)tetrakis(1-pyrazolyl)borate (abbreviated as Fir₆), bis[2-(4′,6′-difluorophenyl)pyridinato-N,C2′]iridium(III)picolinato (abbreviated as Flrpic), bis[2-(3′5′-bistrifluoromethylphenyl)pyridinato-N,C2′]iridium(III)picolinate (abbreviated as Ir(CF₃ppy)₂(pic)), and bis[2-(4′,6′-difluorophenyl)pyridinato-N,C2′]iridium(III)acetylacetonate (abbreviated as Flracac).

As a green phosphorescent emitting material that is usable in the emitting layer, an iridium complex or the like may be used. Specific examples thereof include tris(2-phenylpyridinato-N,C2′)iridium(III) (abbreviated as Ir(ppy)₃), bis(2-phenylpyridinato-N,C2′)iridium(III)acetylacetonate (abbreviated as Ir(ppy)₂(acac)), bis(1,2-diphenyl-1H-benzimidazolato)iridium(III)acetylacetonate (abbreviated as Ir(pbi)₂(acac)), and bis(benzo[h]quinolinato)iridium(III)acetylacetonate (abbreviated as Ir(bzq)₂(acac)).

As a red phosphorescent emitting material that is usable in the emitting layer, a metal complex such as an iridium complex, a platinum complex, a terbium complex or a europium complex may be used. Specific examples thereof include organic metal complexes such as bis[2-(2′-benzo[4,5-a]thienyl)pyridinato-N,C3′]iridium(III)acetylacetonate (abbreviated as Ir(btp)₂(acac)), bis(1-phenylisoquinolinato-N,C2′)iridium(III)acetylacetonate (abbreviated as Ir(piq)₂(acac)), (acetylacetonato)bis[2,3-bis(4-fluorophenyl)quinoxalinato]iridium(III) (abbreviated as Ir(Fdpq)₂(acac)), and platinum (11) 2,3,7,8,12,13,17,18-octaethyl-21H,23H-porphyrin (abbreviated as PtOEP).

In addition, since rare-earth metal complexes such as tris(acetylacetonato) (monophenanthroline)terbium(III) (abbreviated as Tb(acac)₃(Phen)), tris(1,3-diphenyl-1,3-propanedionate) (monophenanthroline)europium(III) (abbreviated as Eu(DBM)₃(Phen)), and tris[1-(2-thenoyl)-3,3,3-trifluoroacetonato](monophenanthroline)europium(III) (abbreviated as Eu(TTA)₃(Phen)) emit light (i.e., electronic transition between different multiplicities) from rare-earth metal ions, said complexes may be used as phosphorescent compounds.

According to one aspect of the present invention, the phosphorescent emitting material is preferably an ortho-metalated complex of a metal atom selected from iridium (Ir), osmium (Os) and platinum (Pt).

The phosphorescent emitting material that is an ortho-metalated complex of a metal atom selected from iridium (Ir), osmium (Os) and platinum (Pt) is preferably a complex represented by the formula (a).

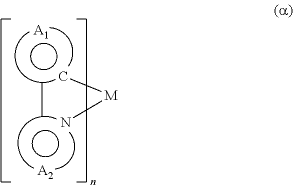

In the formula (a), M is at least one metal selected from osmium, iridium, and platinum, and n is a valence of the metal.

The ring A₁ is a substituted or unsubstituted aryl group including 6 to 24 ring carbon atoms or a heteroaryl group including 5 to 30 ring atoms, and the ring A₂ is a substituted or unsubstituted heteroaryl group including 5 to 30 ring atoms that includes nitrogen as a hetero ring atom.

As examples of the aryl group including 6 to 24 ring carbon atoms in the ring A₁ of the formula (a), the above-described aryl groups in the formula (1) are mentioned.

As examples of the heteroaryl group including 5 to 30 ring atoms in the ring A₁ and ring A₂ of the formula (a), the above-described aryl groups in the formula (1) are mentioned. Substituents that may be contained in the ring A₁ and ring A₂ of the formula (a) are the same as the substituents in the formula (1) described above.

In addition, complexes represented by the formula (a) are preferably complexes represented by the formula (T) or (U).

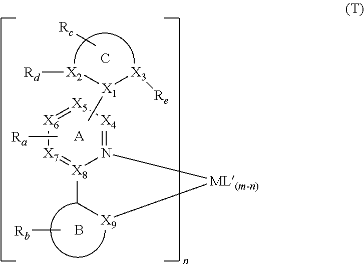

In the formula (T), M is a metal, and the ring B and the ring C are independently an aryl group or a heteroaryl group each including 5 or 6 ring atoms.

The ring A-ring B is a bonding pair of the aryl group or the heteroaryl group, and coordinates to the metal M via a nitrogen atom of the ring A and a sp² hybridized atom of the ring B. The ring A-ring C is a bonding pair of the aryl group or the heteroaryl group.

Rₐ, R_{b}, and R_{c} are independently any one selected from the group consisting of a hydrogen atom, a substituted or unsubstituted alkyl group including 1 to 25 carbon atoms, a substituted or unsubstituted alkoxy group including 1 to 25 carbon atoms, a substituted or unsubstituted amino group, a substituted or unsubstituted alkenyl group including 2 to 25 carbon atoms, a substituted or unsubstituted alkynyl group including 2 to 25 carbon atoms, a substituted or unsubstituted aralkyl group including 7 to 50 carbon atoms, a substituted or unsubstituted aryl group including 6 to 24 ring carbon atoms, and a substituted or unsubstituted heteroaryl group including 5 to 30 ring atoms, and the numbers of Rₐ, R_{b}, and R_{c} are independently one to four.

X₁ to X₉ are independently a carbon atom or a nitrogen atom.

R_{d} and Rₑ are independently any one selected from the group consisting of a hydrogen atom, a substituted or unsubstituted alkyl group including 1 to 25 carbon atoms, a substituted or unsubstituted alkoxy group including 1 to 25 carbon atoms, a substituted or unsubstituted amino group, a substituted or unsubstituted alkenyl group including 2 to 25 carbon atoms, a substituted or unsubstituted alkynyl group including 2 to 25 carbon atoms, a substituted or unsubstituted aralkyl group including 7 to 50 carbon atoms, a substituted or unsubstituted aryl group including 6 to 24 ring carbon atoms, and a substituted or unsubstituted heteroaryl group including 5 to 30 ring atoms, and at least one of R_{c}, R_{d}, and Rₑ binding to the ring C is not a hydrogen atom.

m represents the oxidization state of the metal M and n is 1 or more. L′ is a monoanionic bidentate ligand.

In the formula (T), M is osmium, iridium, platinum, or the like and among them, iridium is preferred.

As the aryl groups including 5 or 6 ring atoms represented by the ring B and ring C, the above-described aryl groups in the formula (1) are mentioned.

As the heteroaryl groups including 5 or 6 ring atoms represented by the ring B and ring C, the above-described heteroaryl groups are mentioned.

As the substituted or unsubstituted alkyl group including 1 to 25 carbon atoms, substituted or unsubstituted alkoxy group including 1 to 25 carbon atoms, substituted or unsubstituted aralkyl group including 7 to 50 carbon atoms, substituted or unsubstituted aryl group including 6 to 24 ring carbon atoms, and substituted or unsubstituted heteroaryl group including 5 to 30 ring atoms represented by R₁, R₂, Rₐ, R_{b}, and Rₑ, groups similar to the groups described above are mentioned.

As the substituted or unsubstituted alkenyl group including 2 to 25 carbon atoms and substituted or unsubstituted alkynyl group including 2 to 25 carbon atoms represented by R₁, R₂, Rₐ, R_{b}, and R_{c}, groups similar to the groups described above are mentioned.

Examples of the monoanionic bidentate ligand represented by L′ include ligands represented by the formula (L′).

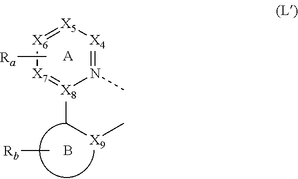

In the formula (L′), X₄ to X₉, Rₐ, and R_{b} are the same as X₄ to X₉, Rₐ, and R_{b} defined in the formula (T) and the preferred aspects thereof are also the same.

A ligand represented by the formula (L′) coordinates to the metal M in the formula (T) via the solid line extending from X₉ to the outside of the ring B and a broken line extending from the nitrogen atom of the ring A to the outside of the ring A.

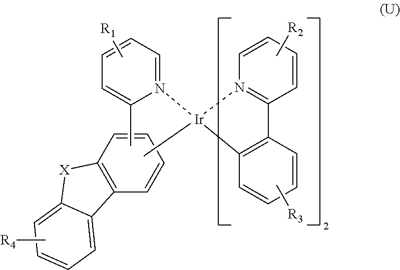

In the formula (U), X is any one selected from the group consisting of NR, an oxygen atom, a sulfur atom, BR, and a selenium atom, and R is a hydrogen atom or a substituted or unsubstituted alkyl group including 1 to 25 carbon atoms.

R₁, R₂, R₃, and R₄ are independently any one selected from the group consisting of a hydrogen atom, a substituted or unsubstituted alkyl group including 1 to 25 carbon atoms, and a substituted or unsubstituted aryl group including 6 to 24 ring carbon atoms. The numbers of R₁, R₂, R₃, and R₄ are independently one to four.

In the formula (U), alkyl groups including 1 to 25 carbon atoms represented by R, R₁, R₂, R₃, and R₄ are the groups described above and the preferred aspects thereof are also the same. In addition, aryl groups including 6 to 24 ring carbon atoms represented by R₁, R₂, R₃, and R₄ are the groups described above and the preferred aspects thereof are also the same.

The following compounds are preferred as complexes represented by the formula (T) or (U), but the complexes are not particularly limited thereto.

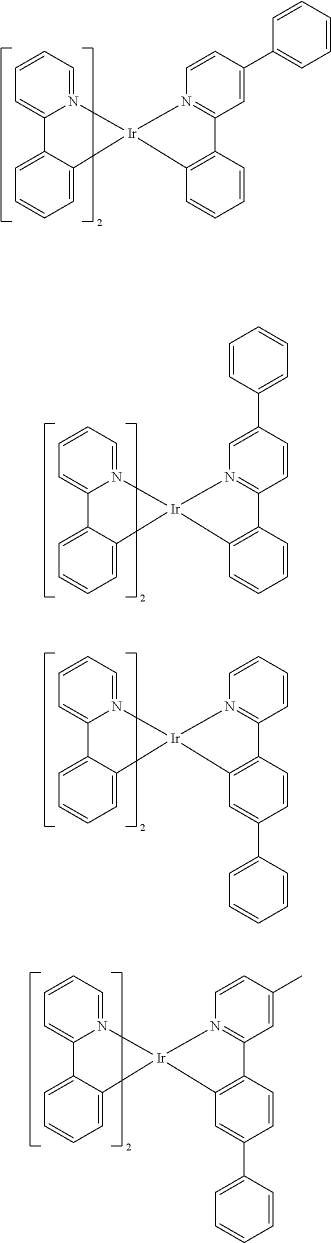

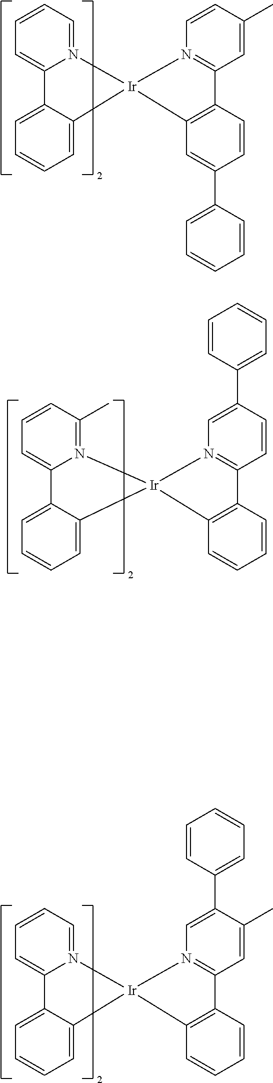

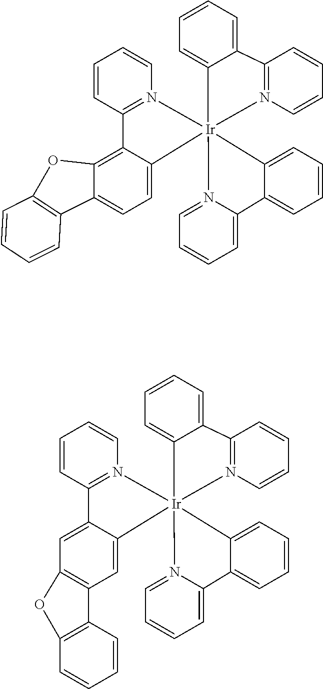

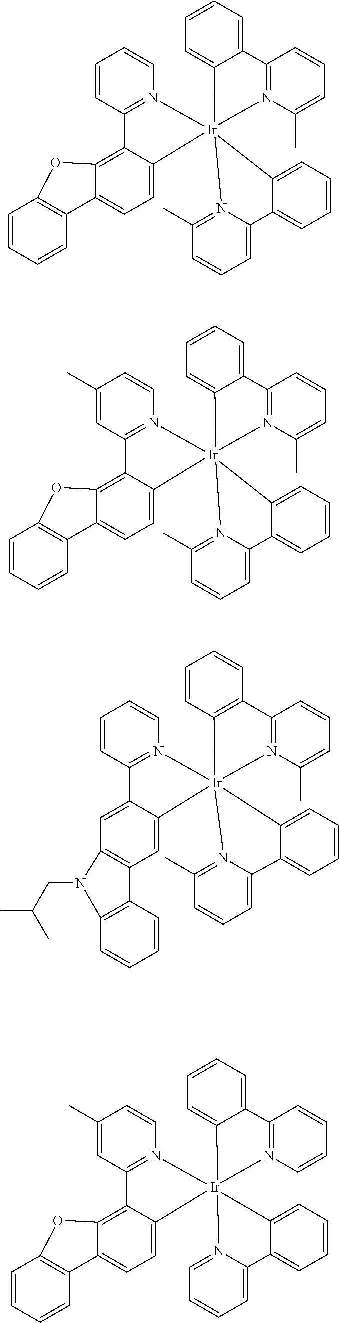

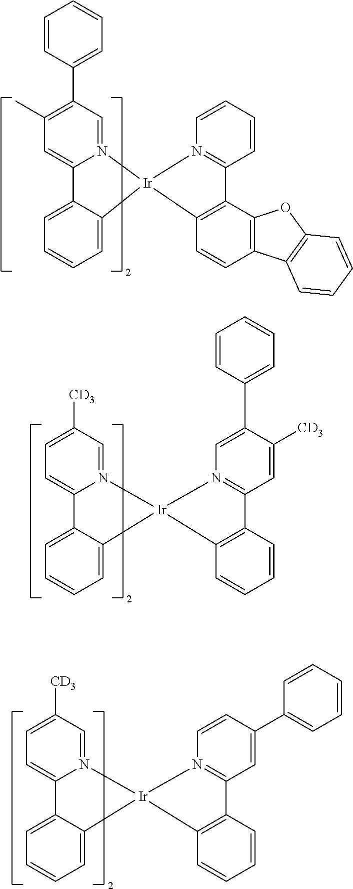

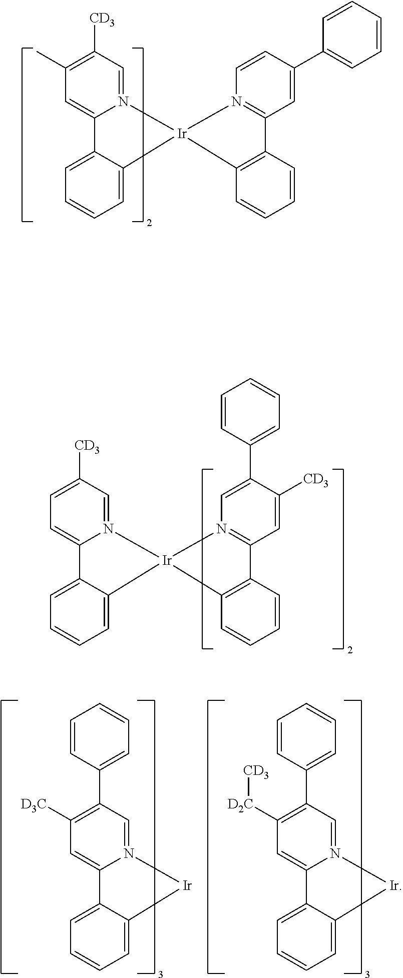

The complexes represented by the formula (a) is not limited to the complexes represented by the formula (T) or (U), and other complexes represented by the following formula (V), (X), (Y), or (Z) may also be used.

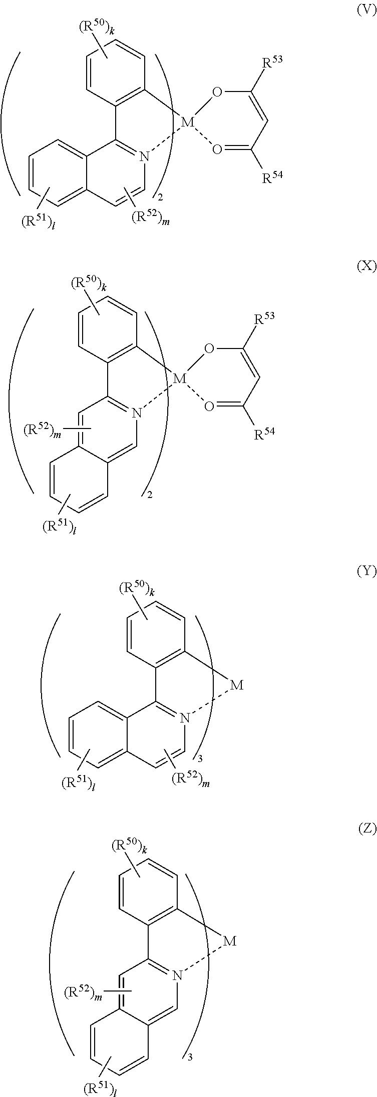

In the formula (V), (X), (Y), or (Z), R⁵⁰ to R⁵⁴ are a hydrogen atom or a substituent, k is an integer of 1 to 4, I is an integer of 1 to 4, and m is an integer of 1 to 2. M is Ir, Os, or Pt. Substituents represented by R⁵⁰ to R⁵⁴ are the same as the substituents described above. The formula (V) is preferably represented by the formula (V-1), and the formula (X) is preferably represented by the formula (X-1) or (X-2). In the following (V-1), (X-1) and (X-2), R⁵⁰, k, and M are the same as R⁵⁰, k, and M described above.

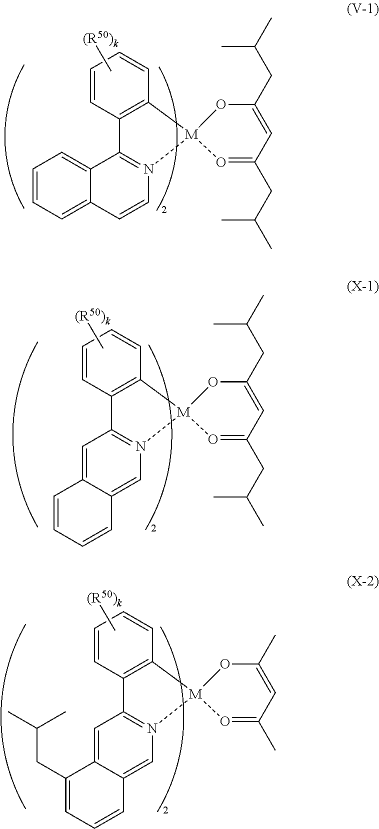

The following are the specific examples of complexes represented by the formula (V), (X), (Y), or (Z), but the complexes are not particularly limited thereto.

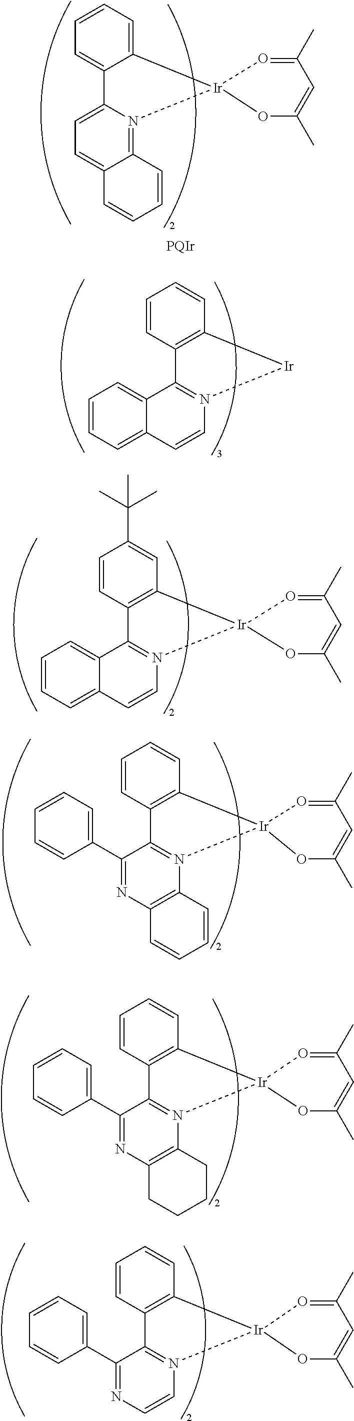

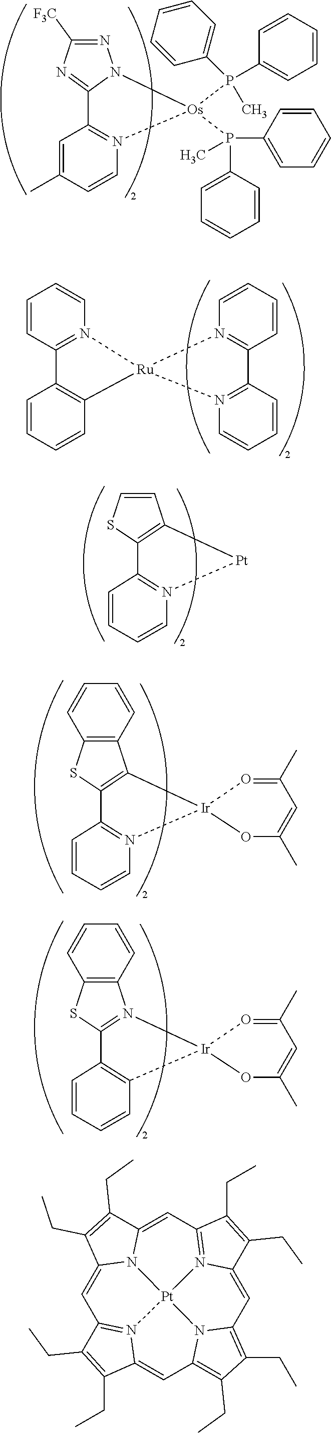

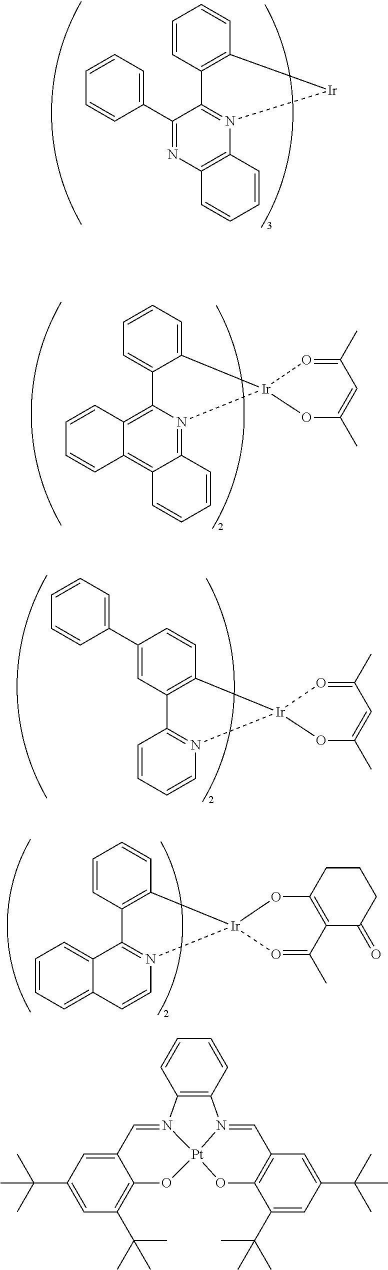

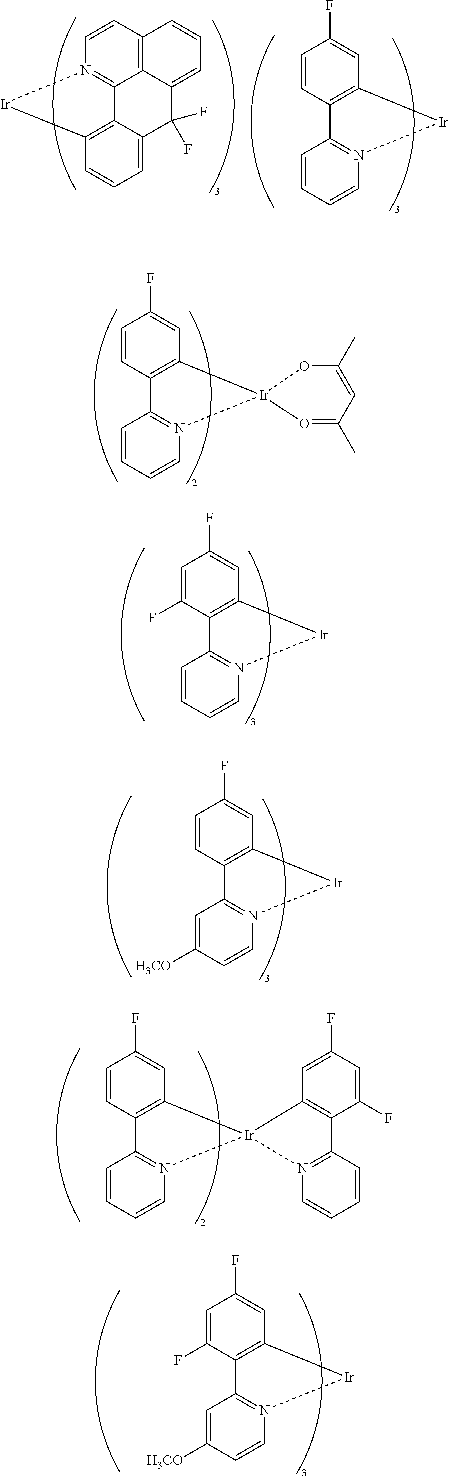

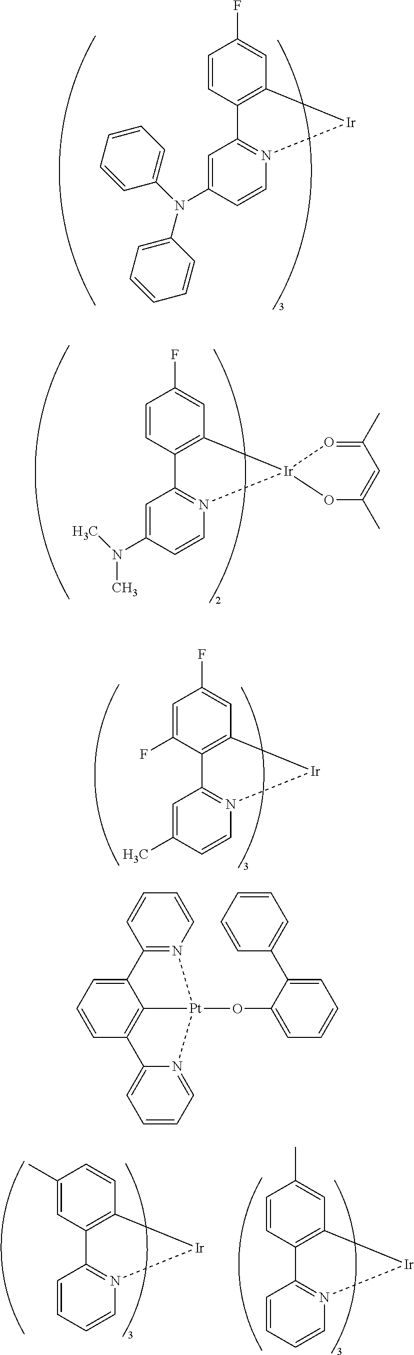

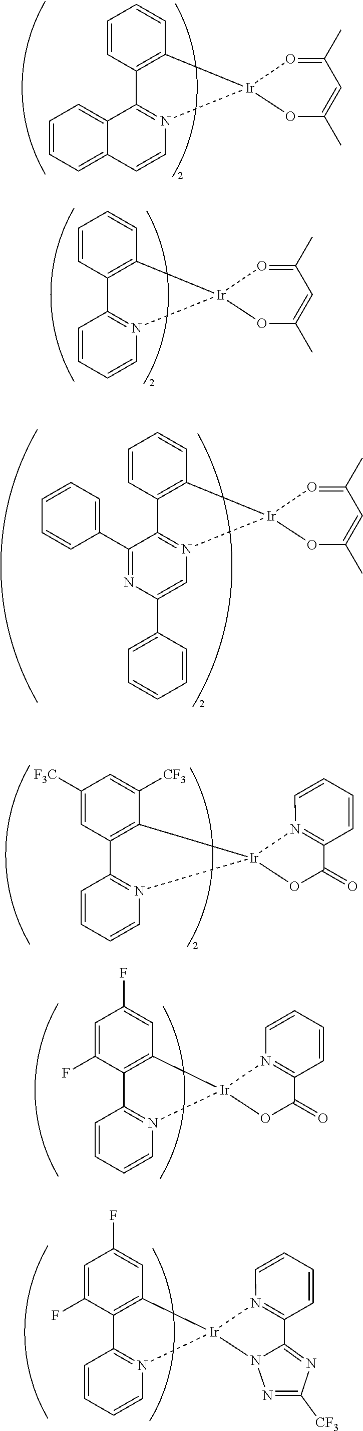

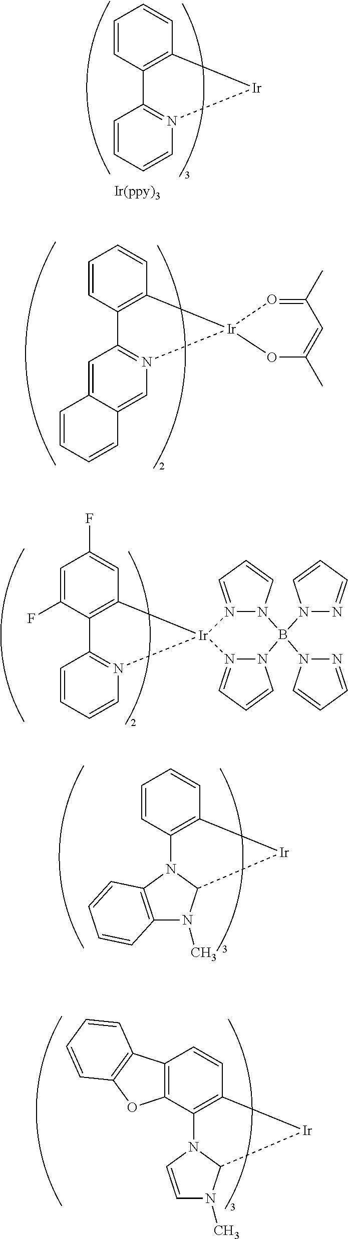

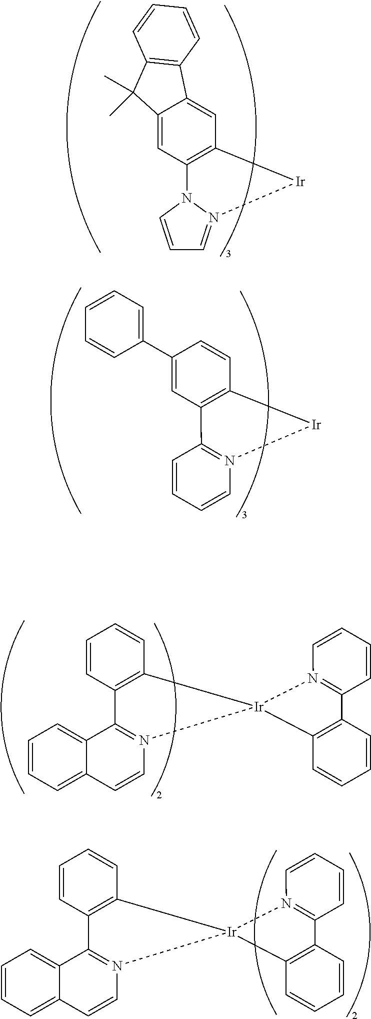

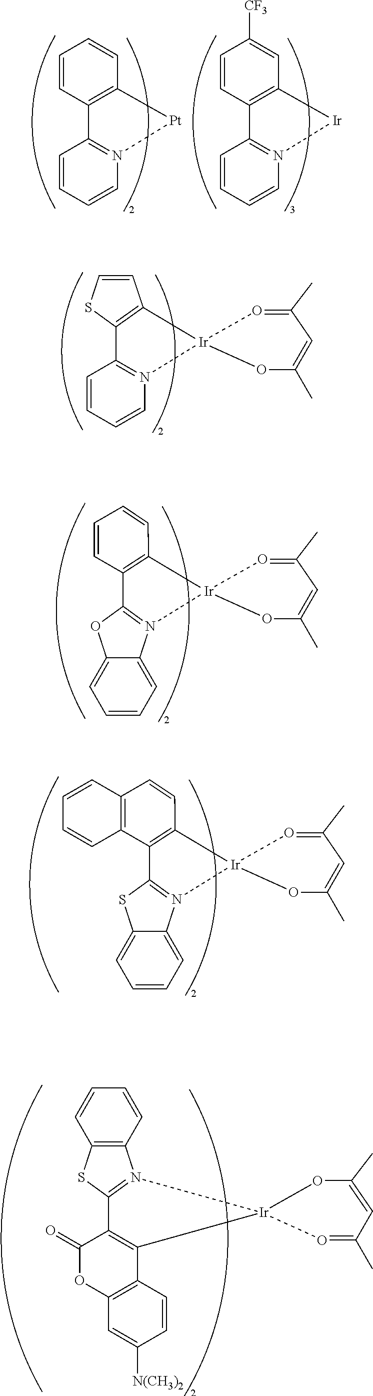

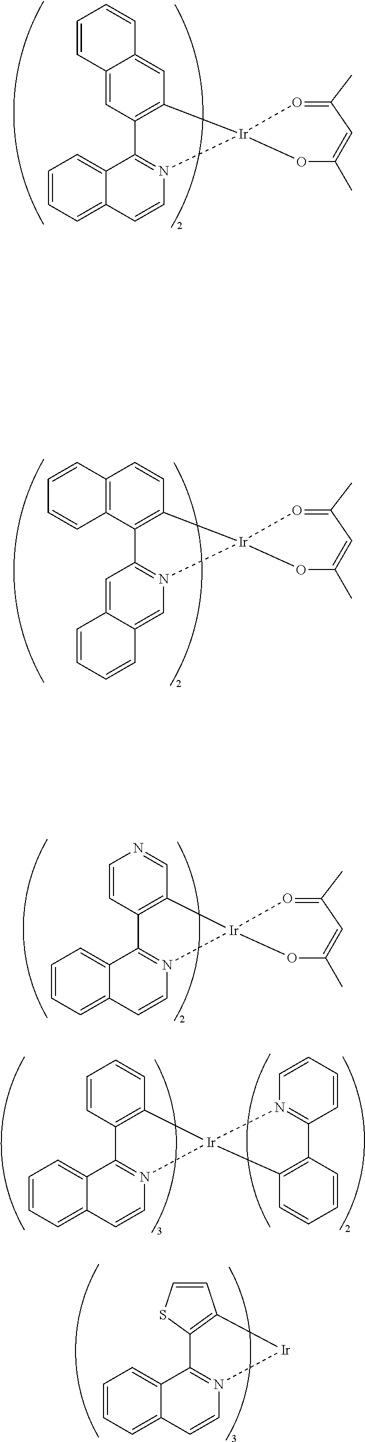

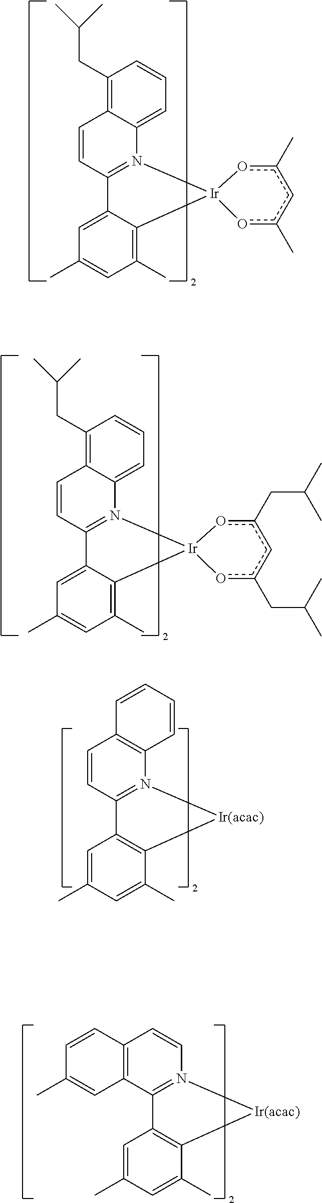

As the phosphorescent emitting materials, iridium complexes represented by the following formula (1) are also preferred.

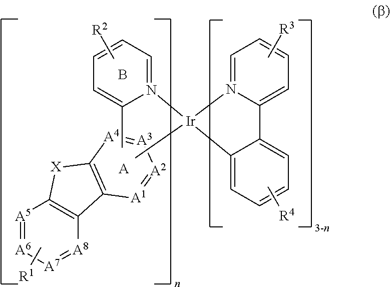

In the formula (1), A¹ to A⁸ include carbon or nitrogen, at least one of A¹ to A⁸ is nitrogen, the ring B is bonded with the ring A via a C—C bond, and iridium (Ir) is bonded with the ring A via an Ir—C bond. It is preferred that only one of A¹ to A⁸ be nitrogen, and it is further preferred that only one of A⁵ to A⁸ be nitrogen.

X is O, S, or Se, and O is preferred.

R¹ to R⁴ are independently mono-, di-, tri-, or tetra-substituted or unsubstituted, adjacent R¹ to R⁴ may be bonded with each other to form a ring, and R¹ to R⁴ are independently hydrogen, halogen, substituted or unsubstituted alkyl including 1 to 25 carbon atoms, substituted or unsubstituted cycloalkyl including 3 to 25 ring carbon atoms, substituted or unsubstituted heteroalkyl including 2 to 25 atoms, substituted or unsubstituted arylalkyl including 7 to 50 carbon atoms, substituted or unsubstituted alkoxy including 1 to 25 carbon atoms, substituted or unsubstituted aryloxy including 6 to 24 ring carbon atoms, substituted or unsubstituted amino, silyl substituted by one or more groups selected from the group consisting of alkyl groups including 1 to 25 carbon atoms and aryl groups including 6 to 24 ring carbon atoms, substituted or unsubstituted alkenyl including 2 to 25 carbon atoms, cycloalkenyl including 3 to 25 ring carbon atoms, heteroalkenyl including 3 to 25 atoms, alkynyl including 2 to 25 carbon atoms, aryl including 6 to 24 ring carbon atoms, heteroaryl including 5 to 30 ring atoms, acyl, carbonyl substituted by one or more groups selected from the group consisting of alkyl groups including 1 to 25 carbon atoms and aryl groups including 6 to 24 ring carbon atoms, carboxylic acid, ester, nitrile, isonitrile, sulfanyl, sulfinyl, sulfonyl, phosphino, and combinations thereof.

It is preferable that R¹ to R⁴ be independently selected from the group consisting of hydrogen, alkyl including 1 to 25 carbon atoms, and combinations thereof. R² and/or R³ is preferably an alkyl group including 1 to 25 carbon atoms, and more preferably, said alkyl group is deuterated or partially deuterated.

n is an integer of 1 to 3 and is preferably 1.

As the iridium complexes represented by the formula (1), iridium complexes represented by the following formula (13-1) are preferred.

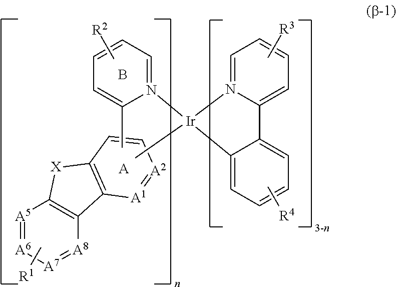

A¹, A², A⁵ to A⁸, X, R¹ to R⁴, and n in the formula (13-1) are as defined in the formula (13).

The following are specific examples of iridium complexes represented by the formula (13) or the formula (13-1), but the complexes are not limited thereto.

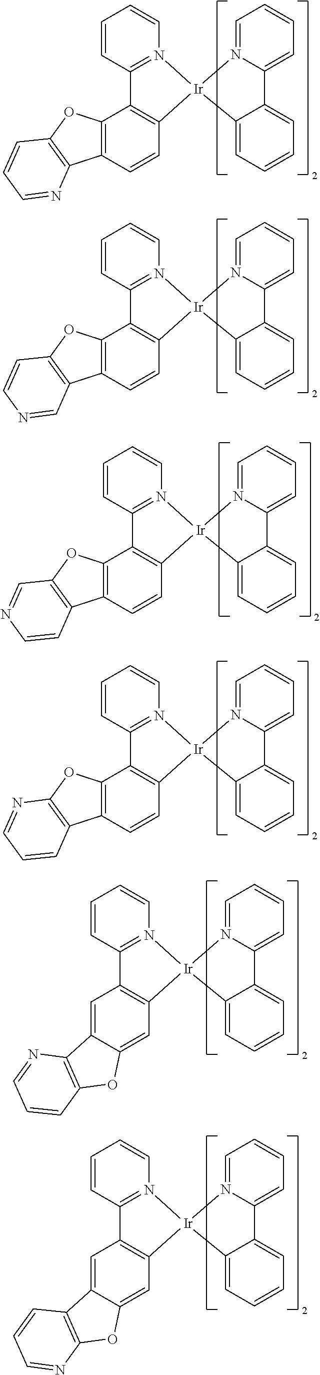

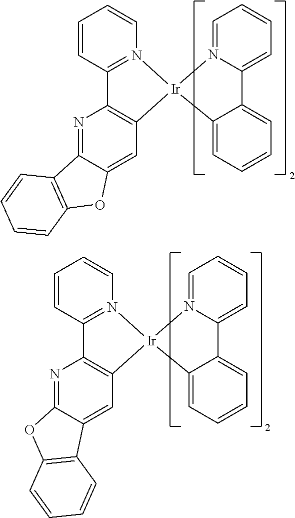

The iridium complexes represented by the formula (3) are preferably iridium complexes represented by the following formula (1-2).

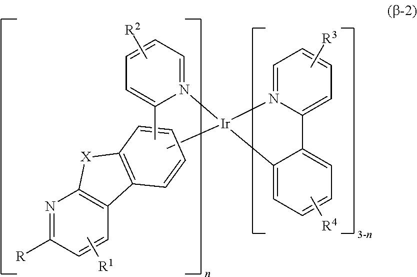

In the formula (1-2), R¹ to R⁴, X and n are as defined in the formula (13).

R is selected from the group consisting of substituted or unsubstituted alkyl including 1 to 25 carbon atoms, substituted or unsubstituted cycloalkyl including 3 to 25 ring carbon atoms, and combinations thereof. R is preferably substituted or unsubstituted alkyl including 1 to 25 carbon atoms, or substituted or unsubstituted cycloalkyl including 3 to 25 ring carbon atoms.

The following are specific examples of iridium complexes represented by the formula (13-2), but the complexes are not limited thereto.

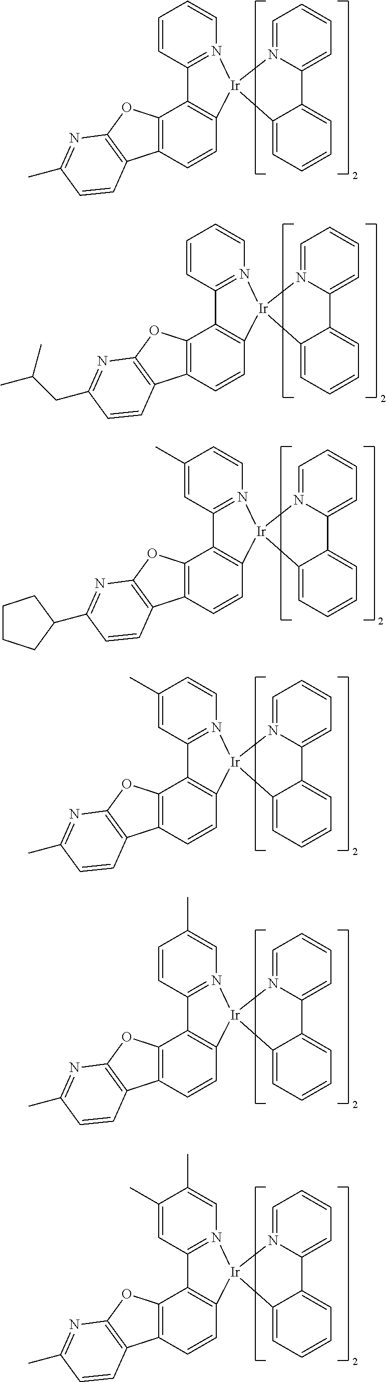

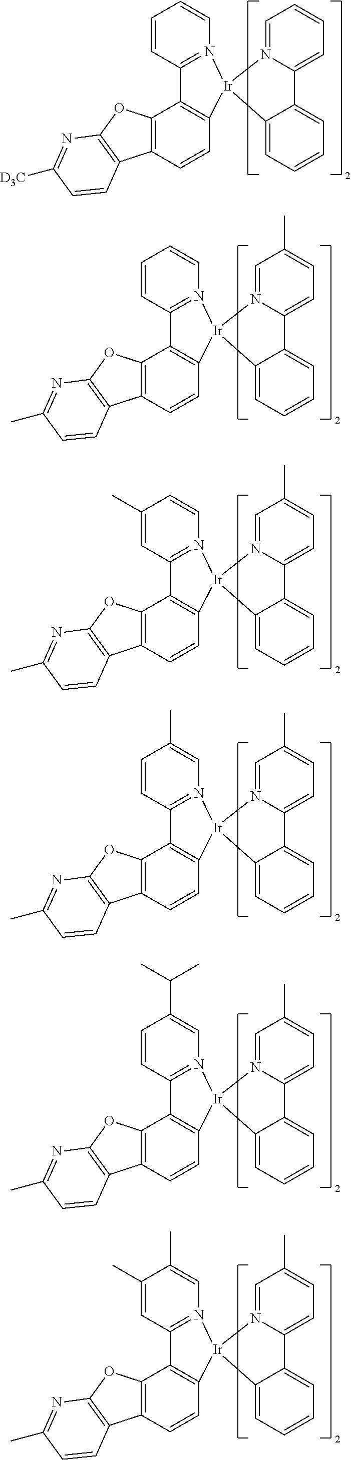

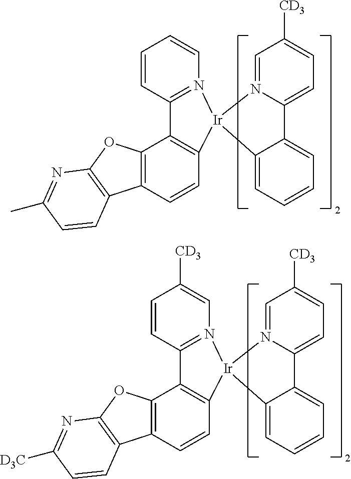

(Host Material of Emitting Layer)

The emitting layer may also have a structure in which the above-described material having a high emitting property (guest material) is dispersed in another material (host material). The material in which the material having a high emitting property is dispersed, is not limited to the compounds represented by the formula (1) or the compounds represented by the formula (2), and various types of materials may be used. It is preferable that materials having a high lowest unoccupied molecular orbital level (LUMO level) and a low highest occupied molecular orbital level (HOMO level) compared with those of the materials having a high emitting property be used.

As the material in which the material having a high emitting property is dispersed (i.e., host material), 1) a metal complex such as an aluminum complex, a beryllium complex, or a zinc complex, 2) a heterocyclic compound such as an oxadiazole derivative, a benzimidazole derivative, or a phenanthroline derivative, 3) a condensed aromatic compound such as a carbazole derivative, an anthracene derivative, a phenanthrene derivative, a pyrene derivative, or a chrysene derivative, 3) an aromatic amine compound such as a triarylamine derivative, or a condensed polycyclic aromatic amine derivative is used.

(Electron Transporting Layer)

An electron transporting layer is a layer containing a material having a high electron-transporting property. In the electron-transporting layer, 1) a metal complex such as an aluminum complex, a beryllium complex, or a zinc complex, 2) a heteroaromatic compound such as an imidazole derivative, a benzimidazole derivative, an azine derivative, a carbazole derivative, or a phenanthroline derivative, 3) a high-molecular compound may be used.

(Electron Injection Layer)

An electron injection layer is a layer containing a material having a high electron-injecting property. In the electron injection layer, an alkali metal, an alkali earth metal, or a compound thereof such as lithium (Li), lithium fluoride (LiF), cesium fluoride (CsF), calcium fluoride (CaF₂), or a lithium oxide (LiOₓ) may be used.

(Cathode)

In a cathode, a metal having a low work function (specifically 3.8 eV or lower), an alloy, an electroconductive compound, a mixture thereof, or the like is preferably used. Specific examples of the cathode materials include elements of the 1^{st} and 2^{nd} groups of the periodic table, namely alkali metals such as lithium (Li) and cesium (Cs) and alkali earth metals such as magnesium (Mg), alloys containing the same (e.g., MgAg, AlLi), rare earth metals and alloys containing the same.

B. Electronic Apparatuses

An electronic apparatus according to one aspect of the present invention is equipped with the organic electroluminescence device according to one aspect of the present invention. The organic electroluminescence device according to one aspect of the present invention is applicable to various electronic apparatuses including flat emitting bodies such as flat panel displays of wall-hanging televisions; backlights of copiers, printers, and liquid crystal displays; light sources of measuring instruments; displaying boards; and marker lamps. In addition, the compounds according to the present invention are usable not only in organic EL devices but also in the field involving xerographic photoreceptors, photoelectric conversion devices, solar cells, image sensors, and the like.

### EXAMPLES

In the following section, the embodiments of the present invention are more specifically explained based on examples and comparative examples, to which the present invention is not particularly limited.

Compounds used in Examples 1 to 4 and Comparative Examples 1 to 6 are collectively shown below.

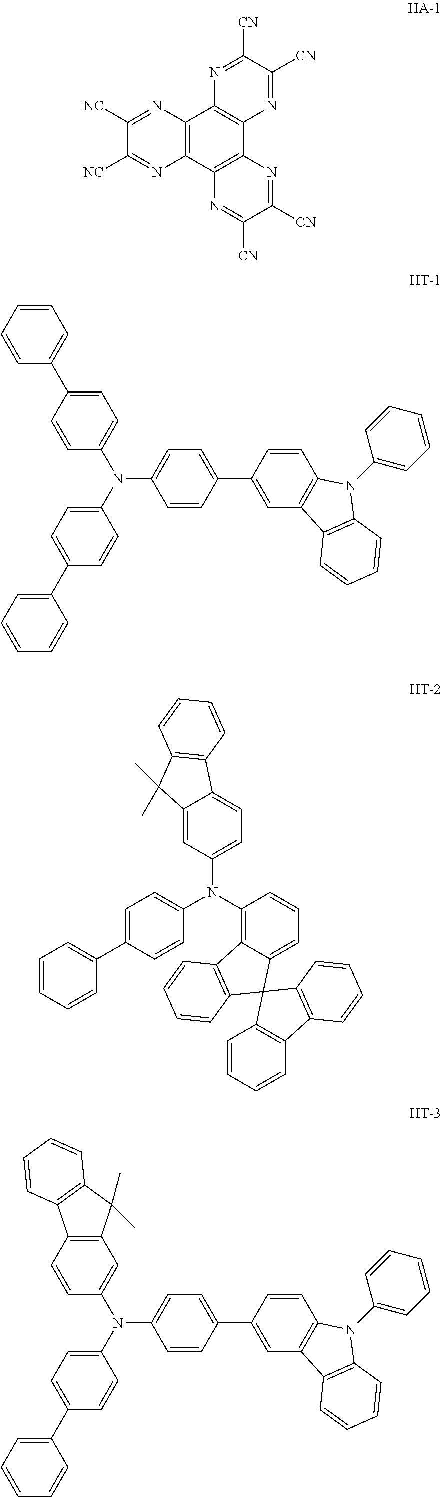 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccccc4)cc3)c3cccc4c3-c3ccccc3C43c4ccccc4-c4ccccc43)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

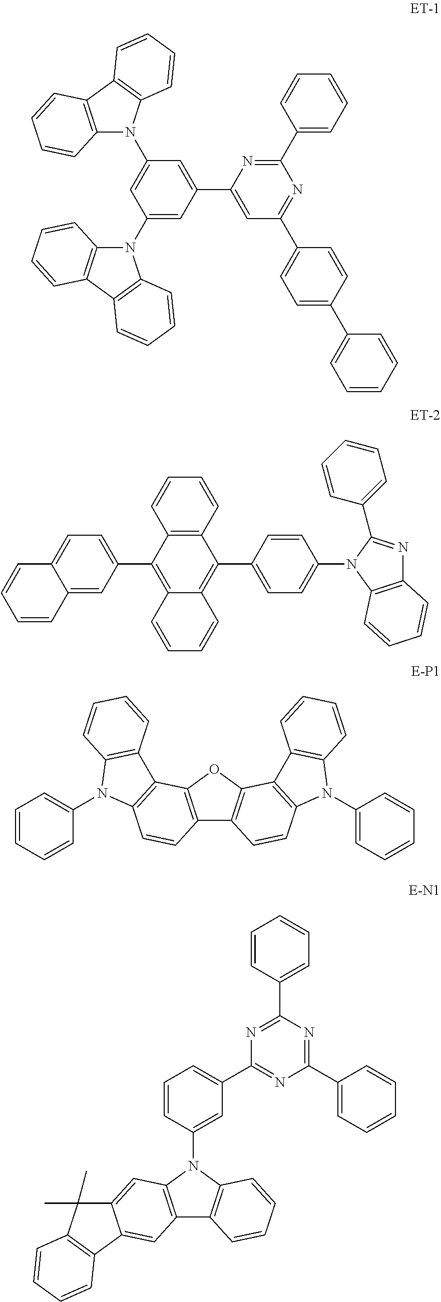 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2nc3ccccc3n2-c2ccc(-c3c4ccccc4c(-c4ccc5ccccc5c4)c4ccccc34)cc2)cc1 ; c1ccc(-n2c3ccccc3c3c4oc5c(ccc6c5c5ccccc5n6-c5ccccc5)c4ccc32)cc1 ; CC1(C)c2ccccc2-c2cc3c4ccccc4n(-c4cccc(-c5nc(-c6ccccc6)nc(-c6ccccc6)n5)c4)c3cc21]

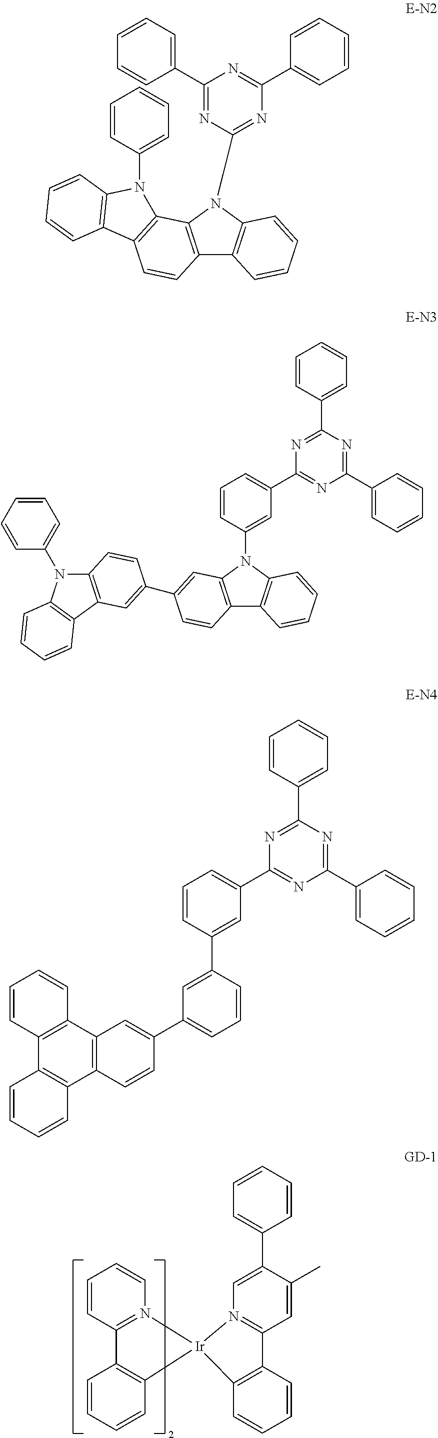

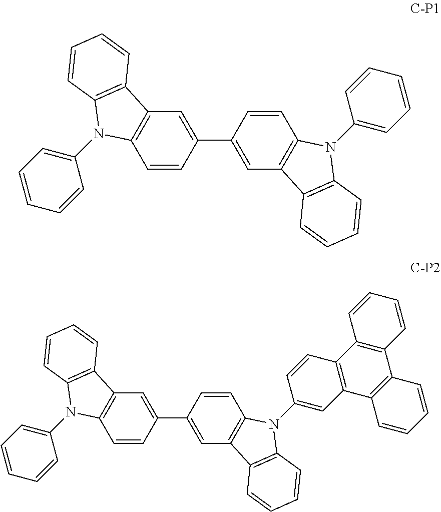 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

### Example 1

A 25 mm×75 mm glass substrate with a thickness of 1.1 mm having an ITO transparent electrode (anode) (produced by GEOMATIC Co., Ltd.) was subjected to ultrasonic cleaning in isopropyl alcohol for 5 minutes, and was thereafter subjected to UV-ozone cleaning for 30 minutes. The ITO film thickness was 130 nm.

The cleaned glass substrate with a transparent electrode line was mounted on a substrate holder in a vacuum vapor deposition apparatus. First, compound HA-1 was vapor deposited on a surface on which the transparent electrode line was formed so as to cover the transparent electrode to form an HA-1 film with a thickness of 5 nm. Thereby a hole injection layer was formed.

Next, compound HT-1 was vapor deposited on the hole injection layer to form an HT-1 film with a thickness of 130 nm. Thereby a first hole transporting layer was formed.

Then, compound HT-2 was vapor deposited on the first hole transporting layer to form an HT-2 film with a thickness of 20 nm. Thereby a second hole transporting layer was formed.

Subsequently, compound E-P1 as a first host material, compound E-N1 as a second host material, and compound GD-1 as a phosphorescent dopant were co-vapor deposited on the hole transporting layer so as to form a film, and thereby an emitting layer with a thickness of 40 nm was formed. The ratio of E-P1 to E-N1 contained in the emitting layer was 33% by mass: 67% by mass, and the concentration of GD-1 therein contained was 5 parts by mass based on 100 parts by mass of the host materials.

Following the emitting layer formation, compound ET-1 and 8-quinolinolato lithium (Liq) in a ratio by mass of 50:50 were co-vapor deposited to form an electron transporting layer with a thickness of 25 nm.

On the electron transporting layer, Liq was vapor deposited to form an electron injection layer with a thickness of 1 nm.

A metal Al was vapor deposited on the electron injection layer to form a metal cathode with a thickness of 80 nm.

The organic EL device of Example 1 was thereby prepared and the lifetime was evaluated. Results are summarized in Table 1.

(Method for Measuring Lifetime (LT₉₇; Time))

A continuous current test (DC) was performed at an initial current density determined to be 10 mA/cm². The time length taken for the luminance to reduce to 97% of the luminance at the time of starting the test was measured. The measured time length was regarded as a lifetime (LT₉₇).

### Examples 2 to 3 and Comparative Examples 1 to 4

Organic EL devices were prepared in the same manner as described in Example 1 except for using host materials shown in Table 1 below, and the lifetimes were evaluated. Results are summarized in Table 1.

TABLE 1

| | First Host | Second Host | LT₉₇ |
|---|---|---|---|
| | Material | Material | [h] |
| Example 1 | E-P1 | E-N1 | 290 |
| Example 2 | E-P1 | E-N2 | 280 |
| Example 3 | E-P1 | E-N3 | 300 |
| Comparative | E-P1 | — | 10 |
| Example 1 | | | |
| Comparative | — | E-N1 | 50 |
| Example 2 | | | |
| Comparative | C-P1 | E-N1 | 200 |
| Example 3 | | | |
| Comparative | C-P2 | E-N1 | 220 |
| Example 4 | | | |

The results in Table 1 show that the device lifetime of each of Examples 1 to 3 in which the emitting layer contains the compound represented by the formula (1) and the compound represented by the formula (2) is significantly improved compared with the lifetimes of the devices in which the emitting layer contains the host material E-P1 alone (Comparative Example 1) and in which the emitting layer contains the host material E-N1 alone. (Comparative Example 2).

In addition, the devices of Examples 1 to 3 exhibit 1.4 to 1.5 times longer lifetimes than the lifetimes of the devices of Comparative Examples 3 and 4 each using a known host material instead of the compound represented by the formula (1).

### Example 4

A 25 mm×75 mm glass substrate with a thickness of 1.1 mm having an ITO transparent electrode (anode) (produced by GEOMATIC Co., Ltd.) was subjected to ultrasonic cleaning in isopropyl alcohol for 5 minutes, and was thereafter subjected to UV-ozone cleaning for 30 minutes. The ITO film thickness was determined to be 130 nm.

The cleaned grass substrate with a transparent electrode line was mounted on a substrate holder in a vacuum vapor deposition apparatus. First, compound HA-1 was vapor deposited on a surface on which the transparent electrode line was formed so as to cover the transparent electrode to form an HA-1 film with a thickness of 5 nm. Thereby a hole injection layer was formed.

Next, compound HT-3 was vapor deposited on the hole injection layer to form an HT-3 film with a thickness of 130 nm. Thereby a first hole transporting layer was formed.

Then, compound HT-2 was vapor deposited on the first hole transporting layer to form an HT-2 film with a thickness of 20 nm. Thereby, a second hole transporting layer was formed.

Subsequently, compound E-P1 as a first host material, compound E-N4 as a second host material, and compound GD-1 as a phosphorescent dopant were co-vapor deposited on the hole transporting layer to form a film, and thereby an emitting layer with a thickness of 40 nm was formed. The ratio of E-P1 to E-N1 contained in the emitting layer was 50% by mass: 50% by mass, and the concentration of GD-1 therein contained was 5% by mass.

Following the emitting layer formation, compound ET-2 and 8-quinolinolato lithium (Liq) in a ratio by mass of 50:50 were co-vapor deposited to form an electron transporting layer with a thickness of 25 nm.

On the electron transporting layer, Liq was vapor deposited to form an electron injection layer with a thickness of 1 nm.

A metal Al was vapor deposited on the electron injection layer to form a metal cathode with a thickness of 80 nm.

The organic EL device of Example 4 was thereby prepared and the lifetime was evaluated in the same manner as described in Example 1.

### Comparative Examples 5 to 6

Organic EL devices were prepared in the same manner as described in Example 4 except for using host materials shown in Table 2 below. Results are summarized in Table 2.

TABLE 2

| | First Host | Second Host | LT₉₇ |
|---|---|---|---|
| | Material | Material | [h] |
| Example 4 | E-P1 | E-N4 | 320 |
| Comparative | C-P1 | E-N4 | 100 |
| Example 5 | | | |
| Comparative | C-P2 | E-N4 | 120 |
| Example 6 | | | |

The results in Table 2 show that the device of Example 4 in which the emitting layer contains the compound represented by the formula (1) and the compound represented by the formula (2) exhibits a remarkably long lifetime that is 2.7 to 3.2 times as long as the lifetimes of the devices of Comparative Examples 5 and 6 each using a known host material instead of the compound represented by the formula (1).

Compounds used in Examples 5 to 18 and Comparative Examples 7 to 9 other than those described above are as shown below.

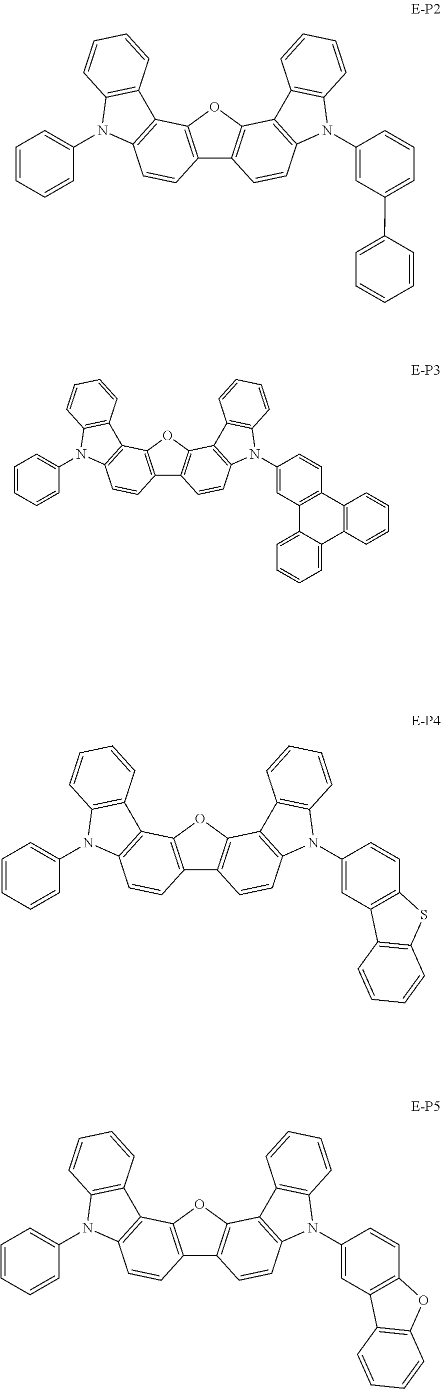 [SMILES: c1ccc(-c2cccc(-n3c4ccccc4c4c5oc6c(ccc7c6c6ccccc6n7-c6ccccc6)c5ccc43)c2)cc1 ; c1ccc(-n2c3ccccc3c3c4oc5c(ccc6c5c5ccccc5n6-c5ccc6c7ccccc7c7ccccc7c6c5)c4ccc32)cc1 ; c1ccc(-n2c3ccccc3c3c4oc5c(ccc6c5c5ccccc5n6-c5ccc6sc7ccccc7c6c5)c4ccc32)cc1 ; c1ccc(-n2c3ccccc3c3c4oc5c(ccc6c5c5ccccc5n6-c5ccc6oc7ccccc7c6c5)c4ccc32)cc1]

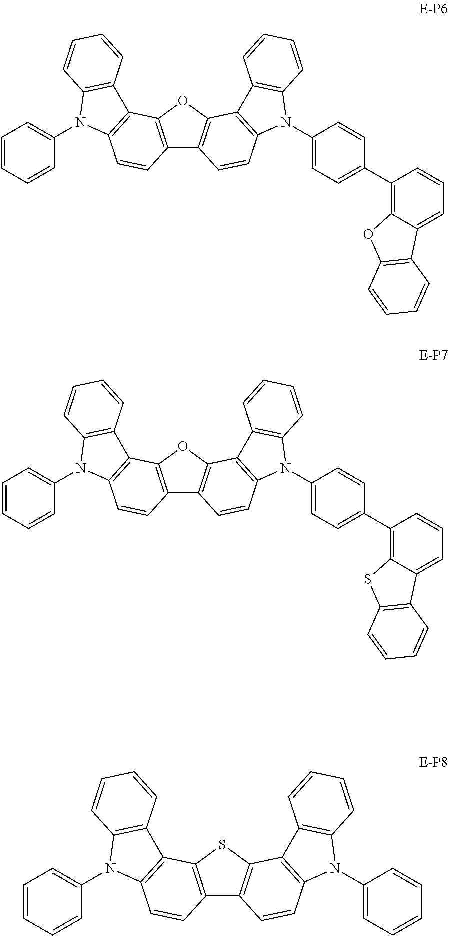 [SMILES: c1ccc(-n2c3ccccc3c3c4oc5c(ccc6c5c5ccccc5n6-c5ccc(-c6cccc7c6oc6ccccc67)cc5)c4ccc32)cc1 ; c1ccc(-n2c3ccccc3c3c4oc5c(ccc6c5c5ccccc5n6-c5ccc(-c6cccc7c6sc6ccccc67)cc5)c4ccc32)cc1 ; c1ccc(-n2c3ccccc3c3c4sc5c(ccc6c5c5ccccc5n6-c5ccccc5)c4ccc32)cc1]

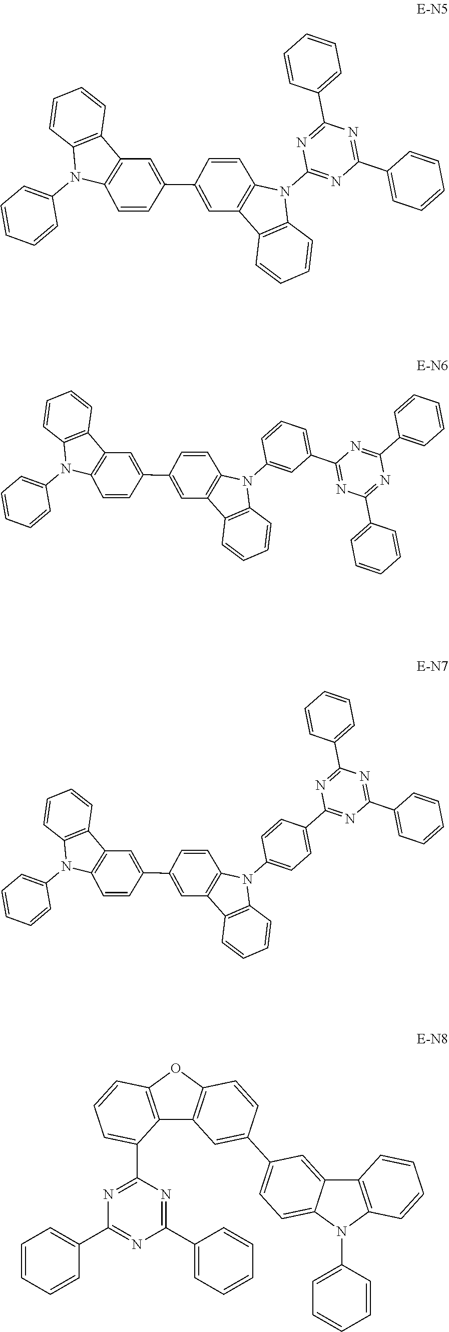 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3cccc4oc5ccc(-c6ccc7c(c6)c6ccccc6n7-c6ccccc6)cc5c34)n2)cc1]

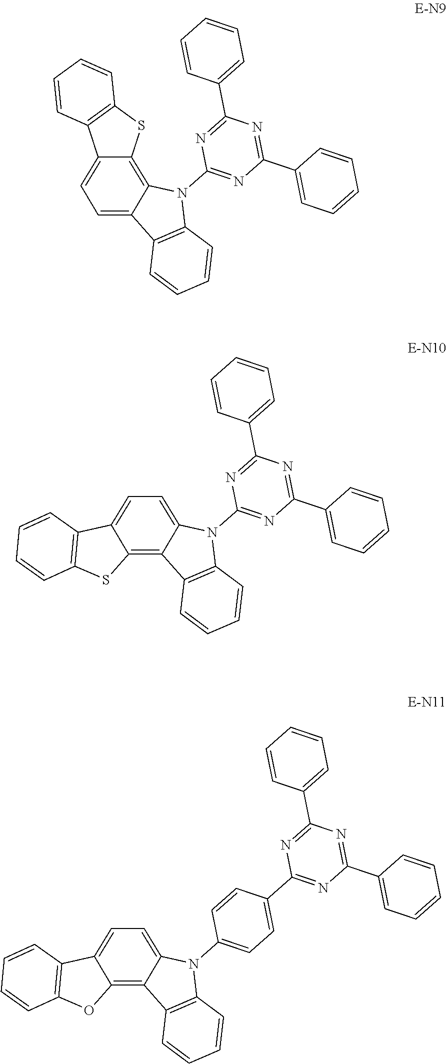 [SMILES: c1ccc(-c2nc(-c3ccccc3)nc(-n3c4ccccc4c4ccc5c6ccccc6sc5c43)n2)cc1 ; c1ccc(-c2nc(-c3ccccc3)nc(-n3c4ccccc4c4c5sc6ccccc6c5ccc43)n2)cc1 ; c1ccc(-c2nc(-c3ccccc3)nc(-c3ccc(-n4c5ccccc5c5c6oc7ccccc7c6ccc54)cc3)n2)cc1]

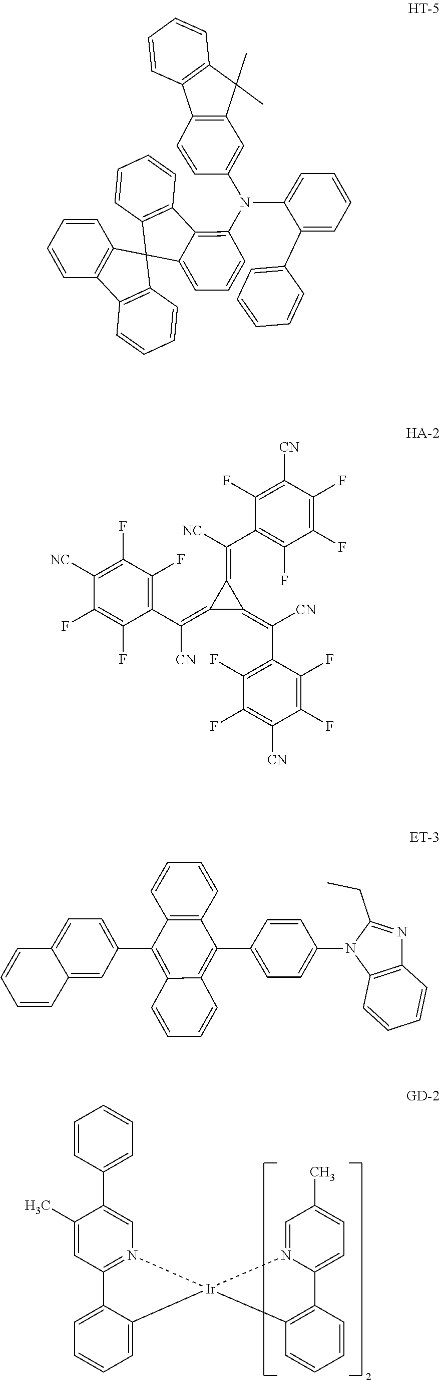

### Examples 5 to 15

Organic EL devices were prepared in the same manner as described in Example 1 except for using host materials shown in Table 3 below, and their lifetimes (LT₉₅) were evaluated as described below. Results are summarized in Table 3.

(Method of Measuring Lifetime (LT₉₅; Time))

A continuous current test (DC) was performed at an initial current density determined to be 10 mA/cm², and the time length taken for the luminance to reduce to 95% of the luminance at the time of starting the test was measured. The measured time length was regarded as a lifetime (LT₉₅).

TABLE 3

| | First Host | Second Host | LT₉₅ |
|---|---|---|---|
| | Material | Material | [h] |
| Example 5 | E-P1 | E-N5 | 280 |
| Example 6 | E-P1 | E-N6 | 300 |
| Example 7 | E-P1 | E-N7 | 310 |
| Example 8 | E-P1 | E-N8 | 290 |
| Example 9 | E-P2 | E-N7 | 320 |
| Example 10 | E-P3 | E-N7 | 320 |
| Example 11 | E-P4 | E-N7 | 320 |
| Example 12 | E-P5 | E-N7 | 320 |
| Example 13 | E-P6 | E-N7 | 320 |
| Example 14 | E-P7 | E-N7 | 320 |
| Example 15 | E-P8 | E-N7 | 310 |

The results in Table 3 show that, as with Examples 1 to 3, the device lifetime of each of Examples 5 to 15 in which the emitting layer contains the compound represented by the formula (1) and the compound represented by the formula (2) is significantly improved compared with the lifetimes of the devices in which the emitting layer contains the host material E-P1 alone (Comparative Example 1) and in which the emitting layer contains the host material E-N1 alone (Comparative Example 2). In addition, the lifetimes of the devices of Examples 5 to 15 are 1.3 to 1.6 times as long as the lifetimes of the devices of Comparative Examples 3 and 4 each using a known host material instead of the compound represented by the formula (1).

### Example 16

A 25 mm×75 mm glass substrate with a thickness of 1.1 mm having an ITO transparent electrode (anode) (produced by GEOMATIC Co., Ltd.) was subjected to ultrasonic cleaning in isopropyl alcohol for 5 minutes, and was thereafter subjected to UV-ozone cleaning for 30 minutes. The ITO film thickness was determined to be 130 nm.

The cleaned glass substrate with a transparent electrode line was mounted on a substrate holder in a vacuum vapor deposition apparatus. First, compounds HT-3 and HA-2 were co-vapor deposited on a surface on which the transparent electrode line was formed so as to cover the transparent electrode to form a film with a thickness of 10 nm. Thereby a hole injection layer was formed. The concentration of compound HA-2 was 3% by mass.

Next, compound HT-3 was vapor deposited on the hole injection layer to form an HT-3 film with a thickness of 110 nm. Thereby a first hole transporting layer was formed.

Then, compound HT-5 was vapor deposited on the first hole transporting layer to form an HT-5 film with a thickness of 35 nm. Thereby, a second hole transporting layer was formed.

Subsequently, compound E-P1 as a first host material, compound E-N9 as a second host material, and compound GD-2 as a phosphorescent dopant were co-vapor deposited on the hole transporting layer to form a film, and thereby an emitting layer with a thickness of 40 nm was formed. The ratio of E-P1 to E-N9 contained in the emitting layer was 33% by mass: 67% by mass, and the concentration of GD-2 therein contained was 5% by mass.

Following the emitting layer formation, a compound ET-3 and Liq in a ratio by mass of 50:50 were co-vapor deposited to form an electron transporting layer with a thickness of 30 nm.

On the electron transporting layer, Liq was vapor deposited to form an electron injection layer with a thickness of 1 nm.

A metal Al was vapor deposited on the electron injection layer to form a metal cathode with a thickness of 80 nm.

The organic EL device of Example 16 was thereby prepared and the lifetime was evaluated in the same manner as described in Example 4. Results are summarized in Table 1.

### Examples 17 and 18, Comparative Examples 7 to 9

Organic EL devices were prepared in the same manner as described in Example 16 except for using host materials shown in Table 4 below.

TABLE 4

| | First Host | Second Host | LT₉₇ |
|---|---|---|---|
| | Material | Material | [h] |
| Example 16 | E-P1 | E-N9 | 100 |
| Example 17 | E-P1 | E-N10 | 90 |
| Example 18 | E-P1 | E-N11 | 90 |
| Comparative | C-P1 | E-N9 | 40 |
| Example 7 | | | |
| Comparative | C-P1 | E-N10 | 30 |
| Example 8 | | | |
| Comparative | C-P1 | E-N11 | 20 |
| Example 9 | | | |

The results in Table 4 show that the device lifetime of each of Examples 16 to 18 in which the emitting layer contains the compound represented by the formula (1) and the compound represented by the formula (2) is 2.3 to 5 times as long as the lifetimes of the devices of Comparative Examples 7 and 8 each using a known host material instead of the compound represented by the formula (1).

Although the embodiments and/or examples of the present invention are described in detail above, the person skilled in the art will easily appreciate that many modifications can be added to these exemplary embodiments and/or examples without substantially deviating from the novel teachings and effects of the present invention. Accordingly, all such modifications are encompassed within the scope of the present invention.

The documents described in the specification and the Japanese patent applications claiming the priority under the Paris Convention to the invention are incorporated herein by reference in its entirety.

1. An organic electroluminescence device comprising an anode, a cathode, and an emitting layer between the anode and the cathode,
wherein the emitting layer comprises a compound represented by the formula (1) and a compound represented by the formula (2): 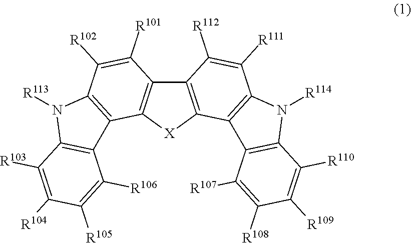
(wherein
R¹⁰¹ to R¹¹² are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, a carbonyl group substituted by one or more groups selected from the group consisting of alkyl groups including 1 to 25 carbon atoms and aryl groups including 6 to 24 ring carbon atoms, or —P(═O)R²⁰R¹²¹,
R¹¹³ and R¹¹⁴ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms,
adjacent R¹⁰¹ to R¹¹⁴ may be bonded with each other to form a ring,
X is an oxygen atom, a sulfur atom, C(R¹¹⁵)(R¹¹⁶), N(R¹¹⁷), or Si (R¹¹⁸)(R¹¹⁹), and
R¹¹⁵ to R¹² are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms); and 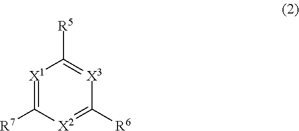
(wherein
X¹ to X³ are independently CR¹¹ or N,
R⁵ to R⁷ and R¹¹ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, a carbonyl group substituted by one or more groups selected from the group consisting of alkyl groups including 1 to 25 carbon atoms and aryl groups including 6 to 24 ring carbon atoms, or —P(═O)R¹⁴R¹⁵,
R¹⁴ and R¹⁵ are independently a substituted or unsubstituted alkyl group including 1 to 25 carbon atoms, a substituted or unsubstituted aryl group including 6 to 24 ring carbon atoms, or a substituted or unsubstituted heterocyclic group including 3 to 30 ring atoms, and
when X¹ to X³ are CR¹¹, they may be bonded with any one of adjacent R⁵ to R⁷ to form a ring).

2. The organic electroluminescence device according to claim 1, wherein X in the formula (1) is an oxygen atom or a sulfur atom.

3. The organic electroluminescence device according to claim 1, wherein R¹¹³ and R¹¹⁴ are independently selected from the group consisting of a substituted or unsubstituted phenyl group, a substituted or unsubstituted biphenyl group, a substituted or unsubstituted naphthyl group, a substituted or unsubstituted terphenyl group, a substituted or unsubstituted phenanthryl group, a substituted or unsubstituted fluoranthenyl group, a substituted or unsubstituted fluorenyl group, a substituted or unsubstituted triphenylenyl group, a substituted or unsubstituted dibenzofuranyl group, a substituted or unsubstituted dibenzothiophenyl group, and a substituted or unsubstituted carbazolyl group.

4. The organic electroluminescence device according to claim 1, wherein the compound represented by the formula (1) is a compound represented by the formula (3): 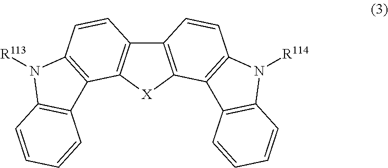
(wherein R¹¹³, R¹¹⁴, and X are as defined in the formula (1)).

5. The organic electroluminescence device according to claim 1, wherein the compound represented by the formula (2) is a compound represented by the following formula (21): 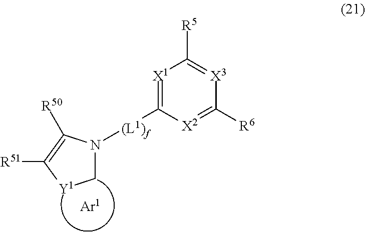
[wherein
X¹ to X³, R⁵, and R⁶ are as defined in the formula (2);
L¹ is a substituted or unsubstituted arylene group including 6 to 24 ring carbon atoms, or a substituted or unsubstituted heteroarylene group including 5 to 30 ring atoms,
f is an integer of 0 to 4; when f is 2 to 4, a plurality of L¹ may be identical with or different from each other, and when f is 0, the group containing X¹ to X³ and the group containing Y¹ are bonded with each other via a single bond,
Y¹ is a carbon atom or a nitrogen atom,
Ar¹ is a substituted or unsubstituted aromatic hydrocarbon ring including 6 to 24 ring carbon atoms that shares a carbon atom and Y¹ with the adjacent nitrogen-containing 5-membered ring and is condensed to the nitrogen-containing 5-membered ring, or a substituted or unsubstituted aromatic heterocyclic ring including 5 to 30 ring atoms that shares a carbon atom and Y¹ with the adjacent nitrogen-containing 5-membered ring and is condensed to the nitrogen-containing 5-membered ring,
R⁵⁰ and R⁵¹ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, or —P(═O)R¹⁴R¹⁵, or R⁵⁰ and R⁵¹ are bonded with each other to form a substituted or unsubstituted aromatic ring group including 6 to 24 ring carbon atoms, and
R¹⁴ and R¹⁵ are as defined in the formula (2)].

6. The organic electroluminescence device according to claim 5, wherein the compound represented by the formula (21) is a compound represented by the formula (22): 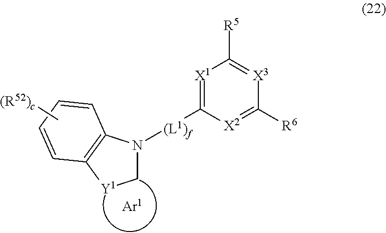
[wherein
X¹ to X³, R⁵, R⁶, L¹, f, Y¹, and Ar¹ are as defined in the formula (21),
R⁵² is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, or —P(═O)R¹⁴R¹⁵,
R¹⁴ and R¹⁵ are as defined in the formula (2), and
c is an integer of 0 to 4; when c is 2 to 4, a plurality of R⁵² may be identical with or different from each other and adjacent R⁵² may be bonded with each other to form a ring].

7. The organic electroluminescence device according to claim 6, wherein the compound represented by the formula (22) is a compound represented by the formula (23): 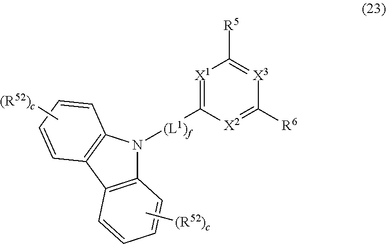
[wherein
X¹ to X³, R⁵, R⁶, L¹, f, R⁵², and c are as defined in the formula (22)].

8. The organic electroluminescence device according to claim 7, wherein the compound represented by the formula (23) is a compound represented by the formula (24): 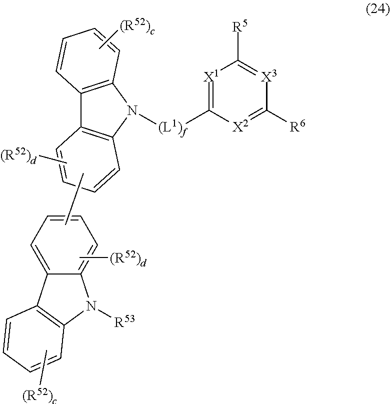
[wherein
X¹ to X³, R⁵, R⁶, L¹, f, R⁵², and c are as defined in the formula (23),
d is an integer of 0 to 3; when d is 2 or 3, a plurality of R⁵² may be identical with or different from each other, and
R⁵³ is a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms].

9. The organic electroluminescence device according to claim 6, wherein the compound represented by the formula (22) is a compound represented by the formula (25): 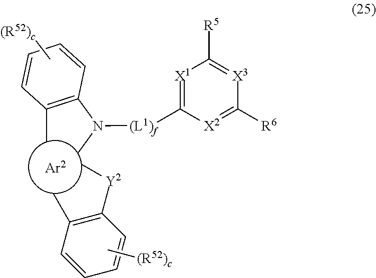
[wherein
X¹ to X³, R⁵, R⁶, L¹, f, R⁵², and c are as defined in the formula (22),
Y² is CR⁵⁴R⁵⁵, NR⁵⁶, an oxygen atom or a sulfur atom,
R⁵⁴ to R⁵⁶ are a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms, and
Ar² is a substituted or unsubstituted aromatic hydrocarbon ring including 6 to 24 ring carbon atoms that shares two carbon atoms of each of adjacent two 5-membered rings and is condensed to the two 5-membered rings, or a substituted or unsubstituted aromatic heterocyclic ring including 5 to 30 ring atoms that shares two carbon atoms of each of adjacent two 5-membered rings and is condensed to the two 5-membered rings].

10. The organic electroluminescence device according to claim 9, wherein the compound represented by the formula (25) is a compound represented by any one of the formulae (26A) to (26F): 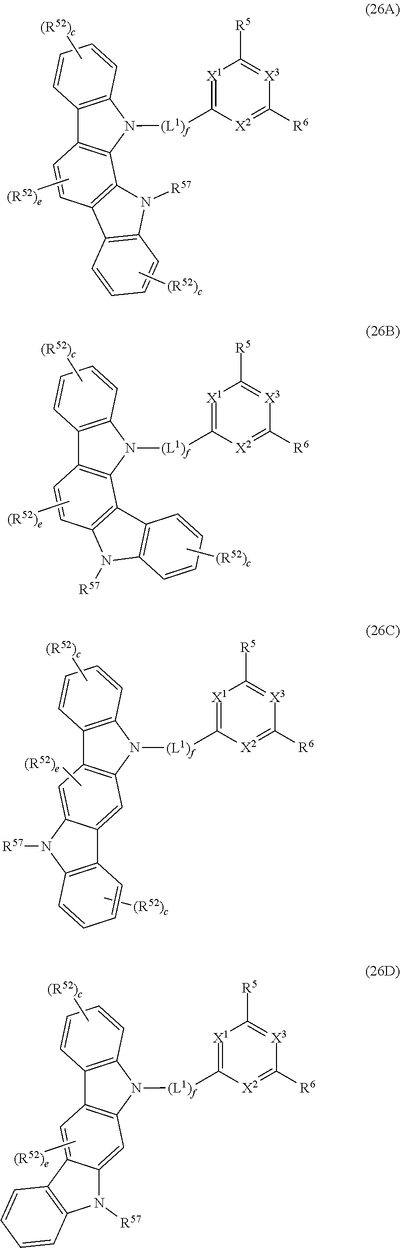 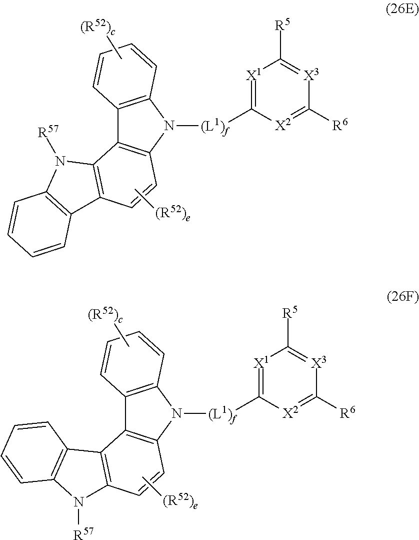
[wherein
X¹ to X³, R⁵, R⁶, L¹, f, R⁵², and c are as defined in the formula (25),
R⁵⁷ is a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms, and
e is an integer of 0 to 2; when e is 2, a plurality of R⁵² may be identical with or different from each other].

11. The organic electroluminescence device according to claim 9, wherein the compound represented by the formula (25) is a compound represented by any one of the formulae (27A) to (27F): 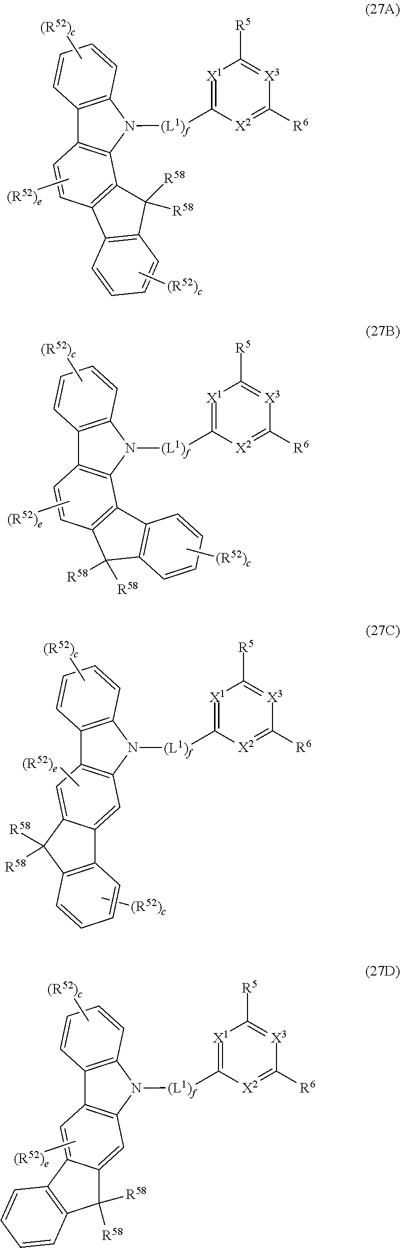 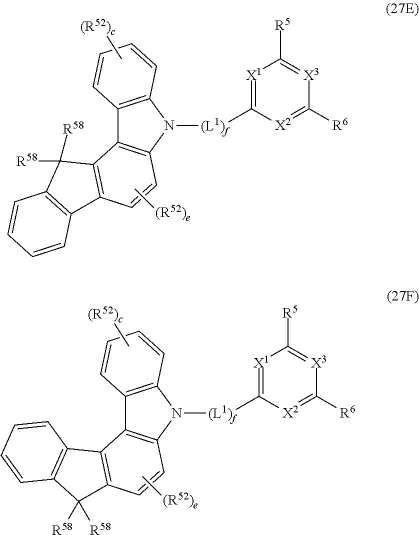
[wherein
X¹ to X³, R⁵, R⁶, L¹, f, R⁵², and c are as defined in the formula (25),
R⁵⁸ is a substituted or unsubstituted alkyl group including 1 to 25 carbon atoms, a substituted or unsubstituted cycloalkyl group including 3 to 25 ring carbon atoms, a substituted or unsubstituted aryl group including 6 to 24 ring carbon atoms, or a substituted or unsubstituted heterocyclic group including 3 to 30 ring atoms, and
e is an integer of 0 to 2; when e is 2, a plurality of R⁵² may be identical with or different from each other].

12. The organic electroluminescence device according to claim 1, wherein the compound represented by the formula (2) is a compound represented by the formula (30): 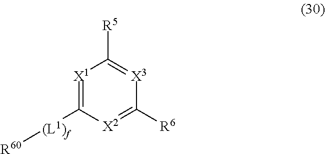
[wherein
X¹ to X³, R⁵, and R⁶ are as defined in the formula (2),
L¹ is a substituted or unsubstituted arylene group including 6 to 24 ring carbon atoms or a substituted or unsubstituted heteroarylene group including 5 to 30 ring atoms,
f is an integer of 0 to 4; when f is 2 to 4, a plurality of L¹ may be identical with or different from each other, and when f is 0, the group containing X¹ to X³ is bonded with R⁶⁰ via a single bond, and
R⁶⁰ is a substituted or unsubstituted condensed aryl group including 10 to 24 ring carbon atoms, or a substituted or unsubstituted condensed heteroaryl group including 9 to 30 ring atoms].

13. The organic electroluminescence device according to claim 12, wherein the compound represented by the formula (30) is a compound represented by the formula (31): 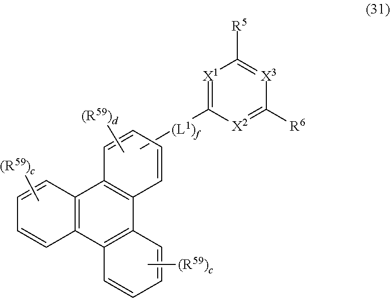
[wherein
X¹ to X³, R⁵, R⁶, L¹, and f are as defined in the formula (30),
R⁵⁹ is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heterocyclic group including 3 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of alkyl groups including 1 to 25 carbon atoms and aryl groups including 6 to 24 ring carbon atoms, a cyano group, or —P(═O)R¹⁴R¹⁵,
R¹⁴ and R¹⁵ are as defined in the formula (2), and
c is an integer of 0 to 4 and d is an integer of 0 to 3; when c is 2 to 4 or d is 2 or 3, a plurality of R⁵⁹ may be identical with or different from each other and adjacent R⁵⁹ may be bonded with each other to form a ring].

14. The organic electroluminescence device according to claim 12, wherein the compound represented by the formula (30) is a compound represented by the formula (32): 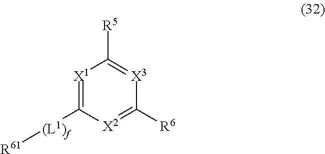
[wherein
X¹ to X³, R⁵, R⁶, L¹, and f are as defined in the formula (30), and
R⁶¹ is a substituted or unsubstituted condensed heteroaryl group including 9 to 30 ring atoms and containing no nitrogen atom].

15. The organic electroluminescence device according to claim 5, wherein f is 0 or 1.

16. The organic electroluminescence device according to claim 1, wherein the emitting layer further contains an emitting material.

17. The organic electroluminescence device according to claim 16, wherein the emitting layer contains a phosphorescent emitting material as the emitting material and the phosphorescent emitting material is an ortho-metalated complex of a metal atom selected from iridium (Ir), osmium (Os), and platinum (Pt).

18. The organic electroluminescence device according to claim 1, further comprising a hole transporting layer between the anode and the emitting layer.

19. The organic electroluminescence device according to claim 1, further comprising an electron transporting layer between the cathode and the emitting layer.

20. An electronic apparatus equipped with the organic electroluminescence device according to claim 1.
